(19) United States
(12) Patent Application Publication
(10) Pub. No.: US 2004/0038339 A1
(43) Pub. Date: Feb. 26, 2004

(54) Multifunctional polypeptides comprising a binding site to an epitope of the nkg2d receptor complex

(76) Inventors: Peter Kufer, Moosburg (DE); Gert Rietmuller, Munich (DE); Ralf Lutterbuse, Munich (DE); Katrin Borschert, Munich (DE); Roman Kischel, Munich (DE); Monika Mayer, Ottobeuren (DE); Robert Hofmeister, Germering (DE)

Correspondence Address: FOLEY AND LARDNER, SUITE 500, 3000 K STREET NW, WASHINGTON, DC, 20007, US

(21) Appl. No.: 10/239,656

(22) PCT Filed: Mar. 26, 2001
(86) PCT No.: PCT/EP01/03414

(30) Foreign Application Priority Data
Mar. 24, 2000 (EP) 00106467.4

(51) Int. Cl. C12P 21/02; C12N 5/06; C07K 14/705; C07H 21/04

(52) U.S. Cl. 435/69.1; 435/320.1; 435/325; 530/350; 536/23.5

(57) ABSTRACT

The present invention relates to a multifunctional polypeptide comprising a first domain comprising a binding site specifically recognizing an extracellular epitope of the NKG2D receptor complex and a second domain having receptor or ligand function. Furthermore, the present invention relates to polynucleotides encoding the multifunctional polypeptide, to vectors comprising said polypeptides and to cells comprising said polypeptides or said vectors. The invention also relates to compositions comprising either of the above recited molecules, alone or in combination, as well as to specific medical uses of the multifunctional polypeptide of the invention.

The present invention relates to a multifunctional polypeptide comprising a first domain comprising a binding site specifically recognizing an extracellular epitope of the NKG2D receptor complex and a second domain having receptor or ligand function. Furthermore, the present invention relates to polynucleotides encoding the multifunctional polypeptide, to vectors comprising said polypeptides and to cells comprising said polynucleotides or said vectors. The invention also relates to compositions comprising either of the above recited molecules, alone or in combination, as well as to specific medical uses of the multifunctional polypeptide of the invention.

Several documents are cited throughout the text of this specification. The disclosure content of each of these documents (including any manufacturer's specifications, instructions etc.) is herewith incorporated by reference.

Many multifunctional polypeptide compounds described in the prior art are bispecific antibodies of varying molecular formats developed for retargeting immune effector cells against malignant or infected target cells, clearing pathogens or autoantibodies from blood circulation, enhancing drug therapy or as vaccines or as carriers e.g. of radioisotopes. Bispecific antibodies designed to redirect the cytotoxic activity of immune effector cells against target cells usually comprise a binding site recognizing a tumor-associated or a viral antigen on the target cells and a second binding site that interacts with a triggering molecule on the effector cells. Among the effector cells recruited in the prior art by bispecific antibody approaches were T-lymphocytes, NK-cells, monocytes and polymorphonuclear neutrophils. Triggering molecules for bispecific antibodies were usually selected from a group of cell surface receptors consisting of CD64, CD16, the α/β-T cell receptor (TCR) and CD3, but also alternative triggering molecules like CD2, CD89, CD32, CD44, CD69 and the TCR-zeta chain were evaluated. Bispecific antibodies capable of redirecting cytotoxic T-lymphocytes (phenotype: CD3⁺/CD56⁻CD8⁺) to target cells either contain a binding site for the TCR, CD3, the zeta-chain or CD2. By engaging one of these triggering molecules, however, antigen specific signaling via the TCR-complex is disturbed since either epitopes of the TCR-complex itself are involved (the TCR, CD3 or the zeta-chain) or in case of CD2 a molecule that directly contributes to the TCR-signal by coaggregation of the src-related protein tyrosine kinase lck, associated with its cytoplasmic tail, with the TCR-complex.

Thus, the technical problem was to provide multifunctional polypeptides that enhance the specific activation of lymphocytes in the direct neighborhood of disease-related cells without interfering with the receptor specificity and/or function of those cytolytic lymphocytes.

The solution to said technical problem is achieved by providing the embodiments characterized in the claims.

Accordingly, the present invention relates to a multifunctional polypeptide comprising a first domain comprising a binding site specifically recognizing an extracellular epitope of the NKG2D receptor complex and a second domain having receptor or ligand function.

The term “multifunctional polypeptide” in connection with the present invention means a polypeptide that effects under suitable (also in vitro) conditions, such as physiological including pathological, such as in vivo or ex vivo conditions at least two, such as three, four, five or six different biological functions. Physiological in vitro conditions include buffered solutions, such as phosphate buffered solutions in the pH range of 5 to 9 and can be further derived from the appended examples. These functions are as specified further below. They include binding of the specified domains with the molecules further specified herein. Binding may subsequently trigger a further biological function including the onset of a cascade, binding to receptors, modulation of signaling pathways or of gene expression and/or influence on apoptotic cell-death. At least two of these domains conferring differing biological functions and preferably the two domains specified herein above do not naturally occur together, i.e. do not naturally occur in this configuration or at all on the same polypeptide or protein or protein complex.

The term “receptor or ligand function” refers to a naturally occurring or non-naturally occurring binding function of a molecule such as a naturally occurring receptor that is preferably located on a cell surface with a fitting ligand; Examples of such receptor/ligand pairs are antibodies/antigens or other members of the Ig superfamily and their corresponding ligands or hormone receptors/hormones or carbohydrate/lectin interactions. Ligands in general, but not exclusively, refer to molecules that have a natural binding partner. In correspondence with the above, they may be antigens or hormones. However, they may also be of non-natural configuration or origin. Receptors/ligands as described above may be of natural origin, of recombinant or (semi) synthetic origin.

NKG2D is a C-type lectin-like NK cell receptor (Houchins (1991) J.Exp.Med. 172:1017) that forms the NKG2D receptor complex together with DAP10 (Wu (1999) Science 285: 730). DAP10 carries an activating sequence motif for Pl₃-kinase in its cytoplasmic domain and acts as signal transduction module for NKG2D that lacks signaling motifs in its cytoplasmic domain. Engagement of this receptor complex triggers a signaling cascade capable of inducing NK cell cytotoxicity. Like other NK cell receptors, the NKG2D receptor complex was also found to be expressed in certain T cell subsets, namely γ/δ-T cells, CD8⁺ α/β-T cells and in a diminishing minority of CD4⁺ α/β-T cells (Bauer (1999) Science 285: 727).

NK cells are dominant effectors of humoral immune responses, that gain antigen specificity through binding of IgG-antibodies to their surface Fcγ-receptor CD16. Thus, CD16 acts as specific antigen receptor enabling antibody-armed NK cells to destroy target cells in an antigen specific manner. T-lymphocytes are the effectors of cellular immune responses, that carry the TCR-complex as specific antigen receptor. The TCR-complex is composed of several invariant chains including the CD3-complex and the zeta chain as well as two variable chains that confer the clonotypic antigen specificity. Depending on the type of variable chains found in the TCR-complex (either α- and β-chain or γ- and δ-chain), T-lymphocytes can be divided into α/β- and γ/δ-T cells. TCR-mediated recognition of target cells by cytotoxic T-lymphocytes i.e.; CD8⁺ α/β-T cells and γ/δ-T cells usually leads to target cell lysis.

The majority of known lymphocyte-directed bispecific antibodies either recruit NK cells or T cells only. NK cells are usually recruited through engagement of CD16, forming the major extracellular part of the Fcγ-receptor IIIA complex, while T cell recruitment is usually mediated through engagement of CD3, an invariant multi-chain component of the T cell receptor (TCR). Bispecific antibodies directed at the zeta chain associated with CD16 on NK cells as well as with the TCR on T cells, are capable of engaging both types of effector lymphocytes (WO00/03016). However, bispecific antibodies directed at the zeta chain, like those directed at CD3, also activate non-cytotoxic CD4⁺ T cells, that in vivo unlike CD8⁺ T cells contribute to undesired side effects e.g. due to systemic cytokine release without essentially contributing to the cytotoxic elimination of target cells.

The NKG2D-specific multifunctional molecules of the invention (which are in preferred embodiments bifunctional molecules comprising said first and second domain referred to above) in contrast to lymphocyte-directed bispecific antibodies known in the prior art are capable of recruiting with exceptional precision the entire range of lymphocytes that naturally carry a cytotoxic phenotype i.e. NK cell, CD8⁺ α/β-T cells and γ/δ-T cells without essentially touching other cell types like CD4⁺ α/β-T cells that are usually not cytotoxic.

The term “recruitment of cytotoxic lymphocytes” as used in the present invention is not limited to redirected lysis but also comprises enhancement of cytotoxicity and T-cell priming.

Thus, the NKG2D-directed molecules of the invention are unique due to their precision of exhaustively but also exclusively recruiting all relevant cytotoxic lymphocytes. In further contrast to lymphocyte-directed bispecific antibodies known in the prior art, the multifunctional molecules of the invention neither directly nor indirectly engage the specific antigen receptors of cytotoxic lymphocytes including the upstream cytoplasmic steps of the corresponding signaling cascades. In other words, function of the T-cell receptor complex is not impaired since the multifunctional polypeptide of the invention does not bind thereto. The signaling cascade downstream of the signal conferred by the T-cell receptor is therefore not affected by the interaction with the multifunctional polypeptide of the invention. As a result, activation and/or proliferation of cytotoxic lymphocytes is selectively supported, that due to their antigen receptor specificity are engaged in a specific immune response against those target cells recognized by the multifunctional molecules of the invention.

Said upstream signaling cascade in T- and NK-cells comprises ITAM polypeptides, Src kinases, ZAP-70/Syk and adaptor proteins such as LAT and SLP-76 responsible for the recruitment of effector molecules of the downstream signaling cascade. The downstream signaling cascade comprises molecules like the Pl3-kinase as well as PLCγ, Grb2, Vav, Cbl and Nck.

By avoiding the engagement of specific antigen receptors and/or the upstream cytoplasmic steps of the corresponding signaling cascades the multifunctional molecules of the invention advantageously interfere to a smaller degree with specific antigen recognition than other lymphocyte-directed bispecific antibodies known in the prior art that e.g. bind to CD16 of the Fcγ-receptor complex on NK cells or to the CD3-component of the TCR-complex on T-lymphocytes. In particular, lymphocyte effector functions mediated by a target cell specific immune response may be overruled through engagement of specific antigen receptors and/or the upstream cytoplasmic steps of the corresponding signaling cascades by bispecific antibodies of the prior art. In contrast, the multifunctional molecules of the invention by engaging the NKG2D receptor complex, which is neither directly associated with specific antigen receptors nor with the upstream steps of their cytoplasmic signaling cascades, are capable of enhancing the activation of those cytotoxic lymphocytes that recognize the same target cell through their specific antigen receptor.

This explains the surprising result described in the appended examples, that an NKG2D-mediated signal can accelerate priming of naive CD8⁺ T-cells even in the presence of

(i) a strong primary signal mediated through engagement of the antigen specific T-cell receptor complex and

(ii) maximum co-stimulation provided by B7-1, the dominant mediator of the second T-cell signal.

Furthermore, it was surprisingly found, that the cytotoxicity of CD8⁺ T-cells and NK-cells triggered by the engagement of the TCR-complex or CD16, respectively, can be enhanced through an NKG2D-mediated signal (Example 6).

Most surprisingly, however, NK- and T-cell cytotoxicity as well as T-cell priming could be even enhanced by NKG2D-directed antibody molecules, which by themselves did not induce any substantial redirected lysis (Examples 5 and 6).

Thus, multifunctional NKG2D-directed polypeptides of the invention with different properties of recruiting cytotoxic lymphocytes may be advantageously selected for different purposes. For example, if pure immunomodulation is required, NKG2D-directed molecules may be preferred, which do not induce re-directed lysis by themselves. However, target cell elimination may be more pronounced when multifunctional NKG2D-directed polypeptides are used that directly trigger lymphocyte cytotoxicity. Moreover, multifunctional NKG2D-directed polypeptides, which differentially recruit CD8⁺ T-cells and NK-cells, may be also preferable for certain applications.

In a preferred embodiment of the method of the present invention said binding site is the binding site of an immunoglobulin chain.

In another preferred embodiment of the method of the present invention said binding site is a natural NKG2D-ligand of said receptor complex.

In a particularly preferred embodiment of the method of the present invention said natural NKG2D-ligand is selected from the group consisting of MIC-A, MIC-B, ULBP1 and ULBP2.

In another preferred embodiment of the method of the present invention said binding site specifically recognizes an extracellular epitope of NKG2D or of DAP10.

Further, in a preferred embodiment of the method of the present invention said receptor or ligand function is an antigen binding site of antibodies or fragments or derivatives thereof against tumor associated antigens, antigens of infective agents or surface markers of sub-populations of cells such as differentiation antigens (CD antigens), natural ligands or receptors or fragments thereof or modifications thereof that interact with tumor associated antigens or surface markers, preferably heregulins, binding to the tumor associated antigens erbB-2, -3 and -4, CD4 that interacts with gp 120 of HIV infected cells or melanocyte stimulating hormone (MSH) that binds to the MSH receptor on melanocytes and tumors derived therefrom (maligned melanomes) or chemokines binding to corresponding chemokine receptors, or MHC molecules or fragments thereof complexed with peptides that bind to T-cell receptors of predefined specificity and thus recognize certain T-cell sub-populations or antigen binding sites of T-cell receptors that specifically interact with predefined MHC peptide complexes, or NKp46 which interacts with haemagglutinin (HA) of influenza virus.

Previous reports indicated that haemagglutinin of Influenza virus can enhance lysis of virus-infected target cells by NK cells as well as activate NK cells directly (Trinchiere, Adv. Immunol. 47 (1989), 187-376 and Alsheikhly, Scand J Immunol., 17 (1983), 129-38 and Alsheikhly, Scand J Immunol. 22 (1985), 529-38). It was shown very recently that a fusion protein consisting of the extracellular domain of NKp46 and the Fc portion of immunoglobulin (Ig) directly bound to haemagglutinin-neuraminidase (HN) glycoprotein expressed on the cell surface of transiently transfected 293 cells (Mandelboim, Nature 409 (2001), 1055-60). Addition of NK Gal cells, a NK line derived from healthy donor peripheral blood lymphocytes induced lysis of HN-transfected 293T cells at least four fold more efficiently than of non-transfected cells (Mandelboim, Nature 409 (2001), 1055-60). The same results were obtained for Influenza virus infected target cells. These data indicate that there is a direct interaction between NKp46 and haemagglutinin, and, further, demonstrate that the mechanism for elimination of Influenza virus infected cells by NK cells is due to the interaction of haemagglutinin (HA) exposed on virus infected cells and NKp46 expressed on the surface of NK cells.

Said receptor or ligand function which is capable of binding to haemagglutinin (HA) of influenza virus is for example derived from monoclonal antibodies like:

a) monoclonal antibody IIB4 binding to residues 155, 159, 188, 189, 193, 198, 199, 201 of influenza A virus strains H3 (Kostolansky, J Gen 81 (2000), 1727-35).

b) monoclonal antibody LMBH6 derived from mice sequentially immunized with bromelain-cleaved haemagglutinin (BHA) from influenza virus A/Aichi/2/68, A/Victoria/3/75 and A/Philippines/2/82 (all H3N2) which recognizes HA of H3N2 influenza A strains (Vanlandschoot, J. Gen. Virol. 79 (1998), 1781-91).

c) monoclonal antibody (MoAb) C179 directed to the stem region of HA-H2 (Lipatov, Acta Virol. 41(1997), 337-40).

In this embodiment, said second domain represents in one preferred embodiment an antigen which is the extracellular part of a surface molecule on cells that are involved in pathologic processes of human diseases like e.g. cancer, viral infections or autoimmune conditions. Elimination or functional silencing of such target cells may be facilitated by in vivo application of the bifunctional molecules of the invention, thus providing therapeutic benefit.

“Fragments” of said antibodies retain the binding specificity of the complete antibodies and include Fab, F(ab′)₂ and Fv fragments. “Derivatives” of said antibodies also retain the binding specificity and include scFv fragments. For further information, see Marlow and Lane, “Antibodies, A Laboratory Mammal” CSH Press, Cold Spring Harbor 1988.

Human cancer diseases may be, for example, cancers like mamma carcinoma, breast cancer, colon carcinoma, pancreas carcinoma, ovarian carcinoma, renal cell and cervix carcinoma, melanoma, small cell lung cancer (SCLC), head and neck cancer, gastric carcinoma, rhabdomyosarcoma, prostate carcinoma, folicular Non-Hodgkin lymphoma (NHL), B cell lymphoma, multiple myeloma, T and B cell leukemias and Hodgkin lymphoma.

Tumor associated antigens comprise pan-carcinoma antigens like CEA (Sundblad Hum. Pathol. 27, (1996) 297-301, Ilantzis Lab. Invest. 76(1997), 703-16), EGFR type I (Nouri, Int. J. Mol. Med. 6 (2000), 495-500) and EpCAM (17-1A/KSA/GA733-2, Balzar J. Mol. Med. 77 (1999), 699-712). EGFR type I is especially overexpressed in glioma and EpCAM in colon carcinoma, MRD (minimal residual disease) and colon carcinoma. EGFR type II (Her-2, ErbB2, Sugano Int. J. Cancer 89 (2000), 329-36) is upregulated in mamma carcinoma and TAG-72 glycoprotein (sTN antigen, Kathan Arch. Pathol. Lab. Med. 124 (2000), 234-9) was found to be expressed in breast cancer. EGFR deletion neoepitope might also play a role as tumor associated antigen (Sampson Proc. Natl. Acad. Sci. USA 97 (2000), 7503-8). The antigens A33 (Ritter Biochem. Biophys. Res. Commun. 236 (1997), 682-6), Lewis-Y (DiCarlo Oncol. Rep. 8 (2001), 387-92), Cora Antigen (CEA-related Cell Adhesion Molecule CEACAM 6, CD66c, NCA-90, Kinugasa Int. J. Cancer 76 (1998), 148-53) and MUC-1 (Mucin) are associated with colon carcinoma (Iida Oncol. Res. 10 (1998), 407-14). Thomsen-Friedenreich-antigen (TF, Gal1β-3GalNAcα1-O-Thr/Ser) is not only found in colon carcinoma (Baldus Cancer 82 (1998), 1019-27) but also in breast cancer (Glinsky Cancer. Res. 60 (2000), 2584-8). Overexpression of Ly-6 (Eshel J. Biol. Chem. 275 (2000), 12833-40) and desmoglein 4 in head and neck cancer and of E-cadherin neoepitope in castric carcinoma was described (Fukudome Int. J. Cancer 88 (2000), 579-83). Prostate-specific membrane antigen (PSMA, Lapidus Prostate 45 (2000), 350-4), prostate stem cell antigen (PSCA, Gu Oncogene 191 (2000) 288-96) and STEAP (Hubert, Proc Natl Acad Sci USA 96 (1999), 14523-8) were associated with prostate cancer. The alpha and gamma subunit of the fetal type acetylcholine receptor (AChR) are specific immunohistochemical markers for rhabdomyosarcoma (RMS, Gattenlohner Diagn. Mol. Pathol. 3 (1998), 129-34).

Association of CD20 with follicular non-Hodgkin lymphoma (Yatabe Blood 95 (2000), 2253-61, Vose Oncology (Huntingt) 2 (2001) 141-7), of CD19 with B-cell lymphoma (Kroft Am. J. Clin. Pathol. 115 (2001), 385-95), of Wue-1 plasma cell antigen with multiple myeloma (Greiner Virchows Arch 437 (2000), 372-9), of CD22 with B cell leukemia (dÁrena Am. J. Hematol. 64 (2000), 275-81), of CD7 with T-cell leukemia (Porwit-MacDonald Leukemia 14 (2000), 816-25) and CD25 with certain T and B cell leukemias had been described (Wu Arch. Pathol. Lab. Med. 124 (2000), 1710-3). CD30 was associated with Hodgkin-lymphoma (Mir Blood 96 (2000), 4307-12). Expression of melanoma chondroitin sulfate proteoglycan (MCSP, Eisenmann Nat. Cell. Biol. 8 (1999), 507-13) and ganglioside GD3 was observed in melanoma (Welte Exp Dermatol 2 (1997), 64-9), while GD3 was also found in small lung cell cancer (SCLC, Brezicka Lung Cancer 1 (2000), 29-36). Expression of ganglioside GD2 was also upregulated in SCLC and in neuroblastoma (Cheresh et al. Cancer Res. 10 (1986), 5112-8). Ovarian carcinoma was associated with Muellerian Inhibitory Substance (MIS) receptor type II (Masiakos Clin. Cancer Res. 11 (1999), 3488-99) and renal as well as cervix carcinoma with expression of carboanhydrase 9 (MN/CAIX, Grabmaier Int. J. Cancer 85 (2000) 865-70). Elevated expression levels of CA 19-9 were found in pancreas carcinoma (Nazli Hepatogastroenterology 47 (2000), 1750-2).

In a most preferred embodiment of the method of the present invention said tumor-associated antigen is selected from the group consisting of Lewis Y, CEA, Muc-1, erbB-2, -3 and -4, Ep-CAM, E-cadherin neoepitope, EGF-receptor (e.g. EGFR type I or EGFR type II), EGFR deletion neoepitope, CA19-9, Muc-1, LeY, TF-, Tn- and sTn-antigen, TAG-72, PSMA, STEAP, Cora antigen, CD7, CD19 and CD20, CD22, CD25, Ig-α and Ig-β, A33 and G250, CD30, MCSP and gp100, CD44-v6, MT-MMPS, (MIS) receptor type II, carboanhydrase 9, F19-antigen, Ly6, desmoglein 4, PSCA, Wue-1, GD2 and GD3 as well as TM4SF-antigens (CD63, L6, CO-29, SAS) or the alpha and gamma subunit of the fetal type acetylcholinreceptor (AChR).

Influenza A, B and C all have a segmented genome, but only certain influenza A subtypes and influenza B cause severe disease in humans. The two major proteins of influenza are the surface glycoproteins-haemagglutinin (HA) and neuraminidase (NA). Haemagglutinin (HA) is involved in the binding and membrane fusion of virus particles to host cells receptors and represents the major target for neutralizing antibodies. Infectivity of influenza depends on the cleavage of HA by specific host proteases, whereas NA is involved in the release of progeny virions from the cell. In birds, the natural hosts of influenza, the virus causes gastrointestinal infection and is transmitted via the faeco-oral route. In mammals, replication of influenza subtypes appears restricted to respiratory epithelial cells but systemic complications can occur.

Rubella virus (RV) is the causative agent of the disease known as measles. Rubella is predominantly a childhood disease and is endemic throughout the world. Natural infections of rubella occur only in humans and are generally mild but complications like polyathralgia can occur in adults. RV infection of women during the first trimester of pregnancy can induce a spectrum of congenital defects in the newborn, known as congenital rubella syndrome (CRS). The pathway whereby RV infection leads to teratogenesis has not been elucidated. Cytopathology in infected fetal tissues suggests necrosis and/or apoptosis as well as inhibition of cell division of precursor cells involved in organogenesis. Rubella virus (RV) virions contain two glycosylated membrane proteins, E1 and E2, that exist as a heterodimer and form the viral spike complexes on the virion surface. Formation of an E1-E2 heterodimer is essential for intracellular transport and cell surface expression of both E1 and E2 (Yang, J. Virol. 72 (1998), 8747-8755). Glycoproteins E1 and E2 expressed on rubella virus infected cells represent target molecules for binding of multifunctional polypeptides of the invention.

Rabies is an important disease in wildlife and dog rabies is still a major public health problem in many developing countries of the world. Rabies virus is transmitted in saliva by animal bites. Most recently bats were found to transmit rabies to humans, often without known exposures. In its classic form, rabies is well recognized, but in cases with a paralytic illness mimicking Landre's Guillain-Barre syndrome diagnosis remains problematically. After exposure rabies can be prevented in non-immunized patients by local wound cleansing and application of rabies vaccine and human rabies-specific immunoglobulins. Rabies glycoprotein RGP is a 505 amino acid type I transmembrane glycoprotein which is important in the biology and pathogenesis of rabies virus infection. RGP also stimulates the development of neutralizing antibodies by the host. N-linked glycosylation is required for immunogenicity and cell surface expression of RGP (Wojczyk, Biochemistry 34 (1995), 2599-2609). RGP of rabies virus expressed on the surface of infected cells represents a target molecules for binding of multifunctional polypeptides of the invention.

In another most preferred embodiment of the method of the present invention said surface marker for an infected cell is selected from the group consisting of viral envelope antigens, e.g. of human retroviruses (HTLV I and II, HIV1 and 2) or human herpes viruses (HSV1 and 2, CMV, EBV), haemagglutinin e.g. of influenza virus (influenza A, B or C), glycoproteins E1 and E2 from rubella virus or RGP of rabies virus.

In another preferred embodiment of the method of the present invention said multifunctional polypeptide is a bi-specific molecule, preferably a bi-specific antibody. For further information about the construction and generation of bi-specific-antibodies, see WO/00/06605.

In a particularly preferred embodiment of the method of the present invention said multifunctional polypeptide is selected from the group consisting of a synthetic, a chimeric and a humanized antibody.

In a further preferred embodiment of the method of the present invention said multifunctional polypeptide is a single-chain.

In an additional preferred embodiment of the method of the present invention said two domains are connected by a polypeptide linker.

In another preferred embodiment of the method of the present invention said first and/or second domain mimic or correspond to a V_{H} and V_{L} region of a natural antibody. Examples of such antibodies comprise human, murine, rat and camel antibodies; antibodies derived from immortalized B-cells (e.g. hybridoma cells), from in vitro section of combinatorial antibody libraries (e.g. by plage display) or from Ig-transgenic mice.

In a further preferred embodiment of the method of the present invention at least one of said domains is a single-chain fragment of the variable region of said antibody.

In an additional preferred embodiment of the method of the present invention said domains are ranged in the order V_{L}NKG2D-V_{H}NKG2D-V_{H}TA-V_{L}-TA, or V_{L}-TA-V_{H}TA-V_{H}NKG2D-V_{L}NKG2D wherein the TA represents a target antigen.

In a particularly preferred embodiment of the method of the present invention said tumor-associated antigen is selected from the group consisting of Lewis Y, CEA, Muc-1, erbB-2, -3 and -4, Ep-CAM, E-cadherin neoepitope, EGF-receptor (e.g. EGFR type I or EGFR type II), EGFR deletion neoepitope, CA19-9, Muc-1, LeY, TF-, Tn- and sTn-antigen, TAG-72, PSMA, STEAP, Cora antigen, CD7, CD19 and CD20, CD22, CD25, Ig-α and Ig-β, A33 and G250, CD30, MCSP and gp100, CD44-v6, MT-MMPs, (MIS) receptor type II, carboanhydrase 9, F19-antigen, Ly6, desmoglein 4, PSCA, Wue-1, GD2 and GD3 as well as TM4SF-antigens (CD63, L6, CO-29, SAS) or the alpha and gamma subunit of the fetal type acetylcholinreceptor (AChR).

In another particularly preferred embodiment of the method of the present invention said polypeptide linker comprises a plurality of glycine, serine and/or alanine residues.

In one further particularly preferred embodiment of the method of the present invention said polypeptide linker comprises a plurality of consecutive copies of an amino acid sequence.

Furthermore, in a particularly preferred embodiment of the method of the present invention said polypeptide linker comprises 1 to 5, 5 to 10 or 10 to 15 amino acid residues.

In a most preferred embodiment of the method of the present invention said polypeptide linker comprises the amino acid sequence Gly-Gly-Gly-Gly-Ser.

In a further preferred embodiment of the method of the present invention said multifunctional polypeptide comprises at least one further domain. Target cell specific immune responses may be further supported by combining the bifunctional molecules of the invention with agents that confer costimulatory or coactivating properties on the target cells.

In one alternative of the combination with additional agents, the molecules of the invention may themselves be equipped with additional functional domains, that may be joined e.g. through another amino acid linker. These additional domains may e.g. mediate CD28- or CD137-engagement (see below). Furthermore, it is envisaged that derivatives of the bifunctional molecules of the invention may be constructed that contain more than one additional functional domain.

Alternatively, the molecules of the invention may be combined with more than one additional agent in a composition e.g. with one of said molecules engaging CD28 and another one engaging CD137.

These agents referred to above may e.g. consist of a binding site specifically recognizing the target cells and the extracellular domain of B7-1 (CD80) or B7-2 (CD86) that interact with CD28 on T- and NK-cells. Alternatively, B7-1 or B7-2 may be replaced by the binding site of a CD28-specific antibody. On T-lymphocytes CD28 acts as costimulatory molecule, which is absolutely required in order to mediate the so-called second signal during primary T cell activation through antigen specific TCR-engagement (=first signal). On NK cells CD28 contributes to the induction of cytotoxicity against target cells expressing CD28 ligands (Chambers (1996) Immunity 5: 311). Other agents that may be advantageously combined with the bifunctional molecules of the invention may consist of a binding site specifically recognizing the target cells and the binding site of a CD137-specific antibody or the extracellular part of the CD137-ligand.

In a most preferred embodiment of the method of the present invention said further domain is linked by covalent or non-covalent bonds.

In another most preferred embodiment of the method of the present invention said at least one further domain comprises an effector molecule having a conformation suitable for biological activity, capable of sequestering an ion or selective binding to a solid support or to a preselected determinant.

In a further most preferred embodiment of the method of the present invention said further domain confers a co-stimulatory and/or a co-activating function.

In a particularly preferred embodiment of the method of the present invention said co-stimulatory function is mediated by a CD28-ligand or a CD137-ligand.

In a further particularly preferred embodiment of the method of the present invention said CD28-ligand or CD137-ligand is B7-1 (CD80), B7-2 (CD86), an aptamer or an antibody or a functional fragment or a functional derivative thereof.

The term “functional fragment” of an antibody is defined as a fragment of an antibody that retains the binding specificity of said antibody (see, for example, Harlow and Lane, “Antibodies, A Laboratory Manual” LSH Press, Cold Spring Harbor, 1988). Examples of such fragments are Fab and F(ab)₂ fragment. “Functional derivatives” of said antibodies retain or essentially retain the binding specificity of said antibody. An example of said derivative is an scFv Fragment.

The invention also relates to a polynucleotide which upon expression encodes a multifunctional polypeptide and/or functional parts of a multifunctional polypeptide of the invention. The term “functional part” is defined in accordance with the invention as to the part that confers the specific function of the first, second or any further domain of a multifunctional polypeptide construct of the invention. The polynucleotide may be DNA, RNA or a derivative thereof such as PNA. Preferably, said polynucleotide is DNA.

Furthermore, the invention relates to a vector comprising the polynucleotide of the present invention.

Many suitable vectors are known to those skilled in molecular biology, the choice of which would depend on the function desired and include plasmids, cosmids, viruses, bacteriophages and other vectors used conventionally in genetic engineering. Methods which are well known to those skilled in the art can be used to construct various plasmids and vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1989), (1994). The vectors of the invention can be reconstituted into liposomes for delivery to target cells.

The vector may be, for example, a phage, plasmid, viral, or retroviral vector. Retroviral vectors may be replication competent or replication defective. In the latter case, viral propagation generally will occur only in complementing host cells. Polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc.

Among preferred eukaryotic vectors are pWLNEO, pSV2CAT, pOG44, pXTI and pSG available from Stratagene; and pSVK3, pBPV, pMSG and pSVL available from Pharmacia. In general, typical cloning vectors include pBscpt sk, PGEM, pUC9, pBR322 and pGBT9. Typical expression vectors include pTRE, pCAL-n-EK, pESP-1, pOP13CAT. Other suitable vectors will be readily apparent to the skilled artisan.

Furthermore, one could use, e.g., a mammalian cell that already comprises in its genome a nucleic acid molecule encoding a polypeptide as described above, but does not express the same or not in an appropriate manner due to, e.g., a weak promoter, and introduce into the mammalian cell a regulatory sequence such as a strong promoter in close proximity to the endogenous nucleic acid molecule encoding said polypeptide so as to induce expression of the same.

In this context the term “regulatory sequence” denotes a nucleic acid molecule that can be used to increase the expression of the polypeptide, due to its integration into the genome of a cell in close proximity to the encoding gene. Such regulatory sequences comprise promoters, enhancers, inactivated silencer intron sequences, 3′UTR and/or 5′UTR coding regions, protein and/or RNA stabilizing elements, nucleic acid molecules encoding a regulatory protein, e.g., a transcription factor, capable of inducing or triggering the expression of the gene or other gene expression control elements which are known to activate gene expression and/or increase the amount of the gene product. The introduction of said regulatory sequence leads to increase and/or induction of expression of polypeptides, resulting in the end in an increased amount of polypeptides in the cell. Thus, the present invention is aiming at providing de novo and/or increased expression of polypeptides.

The invention further relates to a cell transfected with the polynucleotide of the present invention.

The cell of the invention may be a eukaryotic (e.g. yeast, insect or mammalian) or prokaryotic cell. Most preferably, the cell of the invention is a mammalian such as a human cell which may be a member of a cell line e.g. CHO-cells, COS, 293, or Bowes melanoma cells.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis, Basic Methods In Molecular Biology (1986). It is specifically contemplated that polypeptides may in fact be expressed by a host cell lacking a recombinant vector.

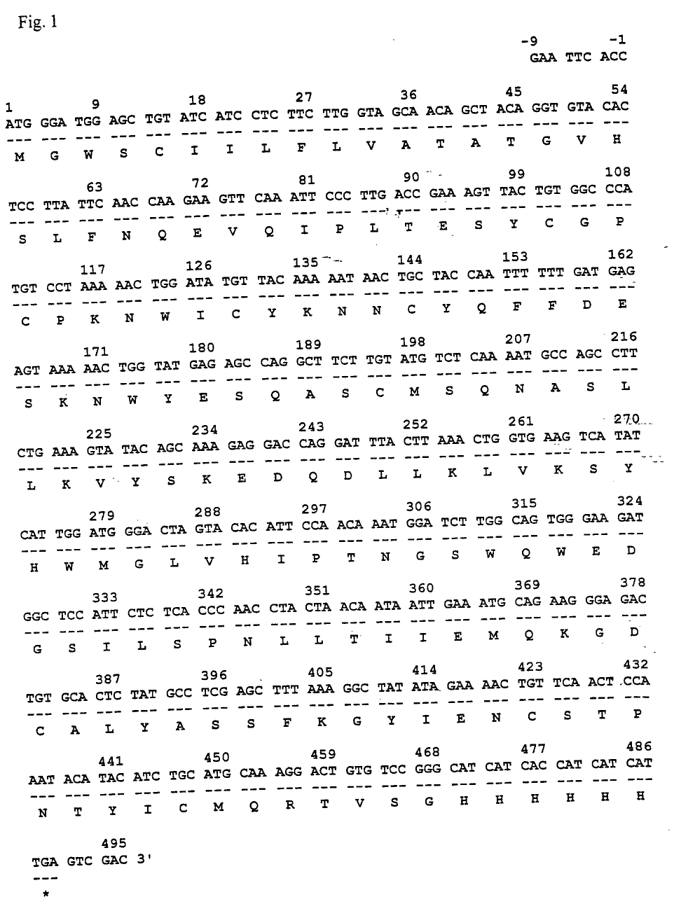

The present invention further provides nucleic acid molecules comprising a polynucleotide encoding upon expression a multifunctional polypeptide and/or functional parts of a multifunctional polypeptide of the invention as described herein and in the appended examples. The nucleic acid sequence of two different fragments of human NKG2D from nucleotides (nt) 64 to 462 and from (nt) 123 to 462 corresponding to amino acid sequences SEQ ID 3 and 4 were PCR-amplified from the cDNA-template shown in FIG. 1. The resulting plasmids VV1-NKG2-D (nt 64-462) and VV1-NKG2-D (nt 123-462) were used to immunize three 6 to 8 weeks old BALB/c mice as mentioned in the appended examples. Resulting lymphocytes were fused with SP2/0 mouse myeloma cells (American Tissue Type Collection, USA) in order to perform hybridoma selection as indicated in the appended examples. Three hybridomas designated 11B2, 8G7 and 6E5 were shown to produce monoclonal antibodies reactive with native NKG2D on the surface of both human CD8⁺ T-lymphocytes and NK-cells (for further information see appended examples). Supernatants of the subclones 11B2D10, 8G7C10 and 6E5A7 were shown to react with NKG2-D on CD56⁺ NK- and CD8⁺ T cells (as demonstrated in the appended examples). These subclones were deposited, at the DSMZ-Deutsche Sammlung von Mikroorganismen und Zellkulturen GmbH, Mascheroder Weg 1b, 38124 Braunschweig, Germany on Mar. 23, 2001, in accordance with the provisions of the Budapest Treaty and given accession number DSM ______, DSM ______ and DSM ______, respectively.

Additionally, the invention relates to a method for the preparation of the multifunctional polypeptide and/or parts of the multifunctional polypeptide of the invention comprising culturing a cell of the present invention and isolating said multifunctional polypeptide or functional parts thereof from the culture as described for example by Mack, 1995, PNAS, 92, 7021.

Polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Depending upon the host employed in a recombinant production procedure, the polypeptides may be glycosylated or may be non-glycosylated. In addition, polypeptides may also include an initial (modified) methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

It is also to be understood that the proteins can be expressed in a cell free system using for example in vitro translation assays known in the art.

The term “expression” means the production of a protein or nucleotide sequence in the cell. However, said term also includes expression of the protein in a cell-free system. It includes transcription into an RNA product, post-transcriptional modification and/or translation to a protein product or polypeptide from a DNA encoding that product, as well as possible post-translational modifications; see also supra. Depending on the specific constructs and conditions used, the protein may be recovered from the cells, from the culture medium or from both. The terms “protein” and “polypeptide” used in this application are interchangeable. “Polypeptide” refers to a polymer of amino acids (amino acid sequence) and does not refer to a specific length of the molecule. Thus peptides and oligopeptides are included within the definition of polypeptide. This term does also refer to or include post-translational modifications of the polypeptide, for example, glycosylations, acetylations, phosphorylations and the like; see also supra. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring. For example, it is well known by the person skilled in the art that it is not only possible to express a native protein but also to express the protein as fusion polypeptides or to add signal sequences directing the protein to specific compartments of the host cell, e.g., ensuring secretion of the protein into the culture medium, etc. The protein of the invention may also be expressed as a recombinant protein with one (polypeptide) or more additional polypeptide domains added to facilitate protein purification. Such purification facilitating domains include, but are not limited to, metal chelating peptides such as histidine-tryptophan modules that allow purification immobilized metals, protein A domains that allow purification on immobilized immunoglobulin, and the domain utilized in the FLAGS extension/affinity purification system (Immunex Corp, Seattle, Wash.). The inclusion of a cleavable linker sequences such as Factor XA or enterokinase (Invitrogen, San Diego, Calif.) between the purification domain and the protein of interest is useful to facilitate purification. One such expression vector provides for expression of a fusion protein compromising a cell cycle interacting protein and contains nucleic acid encoding 6 histidine residues followed by thioredoxin and an enterokinase cleavage site. The histidine residues facilitate purification on IMIAC (immobilized metal ion affinity chromatography as described in Porath, Protein Expression and Purification 3 (1992), 263-281) while the enterokinase cleavage site provides a means for purifying the protein from the fusion protein. In addition to recombinant production, fragments of the protein of the invention may be produced by direct peptide synthesis using solid-phase techniques (cf Stewart et al (1969) Solid Phase Peptide Synthesis, W H Freeman Co, San Francisco; Merrifield, J. Am. Chem. Soc. 85 (1963), 2149-2154). In vitro protein synthesis may be performed using manual techniques or by automation. Automated synthesis may be achieved, for example, using Applied Biosystems 431A Peptide Synthesizer (Perkin Elmer, Foster City, Calif.) in accordance with the instructions provided by the manufacturer. Various fragments of the polypeptide of the invention may be chemically synthesized and/or modified separately and combined using chemical methods to produce the full length molecule. Once expressed or synthesized, the protein of the present invention can be purified according to standard procedures of the art, including ammonium sulfate precipitation, affinity columns, column chromatography, gel electrophoresis and the like; see, Scopes, “Protein Purification”, Springer-Verlag, N.Y. (1982). Substantially pure proteins of at least about 90 to 95% homogeneity are preferred, and 98 to 99% or more homogeneity are most preferred, for pharmaceutical uses. Once purified, partially or to homogeneity as desired, the proteins may then be used therapeutically (including extracorporeally) or in developing and performing assay procedures.

The invention also relates to a composition comprising the polypeptide of the present invention, the polynucleotide of the invention or the vector of the present invention.

In a preferred embodiment of the composition of the present invention said composition further comprises a molecule conferring a co-stimulatory and/or co-activating function.

In this embodiment, the composition may comprise a multifunctional polypeptide that comprises or does not comprise said further domain as defined herein above. If the multifunctional polypeptide comprises a further domain that confers co-stimulatory and/or co-activating function, then said further molecule comprised in the composition of the invention may have the same or a different co-stimulatory and/or co-activating function.

In said composition, the comprised ingredients are packaged together as separately in one or more containers such as vials, preferably under sterile conditions, optionally in buffers or aqueous solutions, some of which are further specified herein below.

In a particularly preferred embodiment of the composition of the present invention said co-stimulatory function is mediated by a CD28-ligand or a CD137-ligand.

In another particularly preferred embodiment of the composition of the present invention said CD28-ligand or CD137-ligand is B7-1 (CD80), B7-2 (CD86), an aptamer or an antibody or a functional fragment or a functional derivative thereof.

In a further preferred embodiment of the composition of the present invention said composition is a pharmaceutical composition optionally further comprising a pharmaceutically acceptable carrier.

The compositions can also include, depending on the formulation desired, pharmaceutically acceptable, usually sterile, non-toxic carriers or diluents, which are defined as vehicles commonly used to formulate pharmaceutical compositions for animal or human administration. The diluent is selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. A therapeutically effective dose refers to that amount of protein or its antibodies, antagonists, or inhibitors which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

Further examples of suitable pharmaceutical carriers are well known in the art and include phosphate buffered saline solutions, water, emulsions, such as oilwater emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 pg to 10 mg units per day. If the regimen is a continuous infusion, it should also be in the range of 0,1 μg to 10 mg units per kilogram of body weight per minute, respectively.

The daily oral dosage regimen will preferably be from about 0.1 to about 80 mg/kg of total body weight, preferably from about 0.2 to 30 mg/kg, more preferably from about 0.5 mg to 15 mg. The daily parenteral dosage regimen about 0.1 μg/kg to about 100 mg/kg of total body weight, preferably from about 0.3 μg/kg to about 10 mg/kg, and more preferably from about 1 μg/kg to 1 mg/kg. The daily topical dosage regimen will preferably be from 0.1 mg to 150 mg, administered one to four, preferably two to three times daily. The daily inhalation dosage regimen will preferably be from about 0.01 mg/kg to about 1 mg/kg per day.

Progress can be monitored by periodic assessment. Dosages will vary but a preferred dosage for intravenous administration of DNA is from approximately 10⁶ to 10¹² copies of the DNA molecule. DNA may also be administered directly to the target site, e.g., by biolistic delivery to an internal or external target site or by catheter to a site in an artery. The compositions comprising, e.g., the polynucleotide, nucleic acid molecule, polypeptide, antibody, compound drug, pro-drug or pharmaceutically acceptable salts thereof may conveniently be administered by any of the routes conventionally used for drug administration, for instance, orally, topically, parenterally or by inhalation. Acceptable salts comprise acetate, methylester, HCl, sulfate, chloride and the like. The drugs may be administered in conventional dosage forms prepared by combining the drugs with standard pharmaceutical carriers according to conventional procedures. The drugs and pro-drugs identified and obtained in accordance with the present invention may also be administered in conventional dosages in combination with a known, second therapeutically active compound. Such therapeutically active compounds comprise, for example, those mentioned above. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof. The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known in the art, such as glyceryl mono-stearate or glyceryl distearate alone or with a wax. A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1 g. When a liquid carrier is used, the preparation will be in the form of a syrup, emulsion, soft gelatin capsule, sterile injectable liquid such as an ampule or nonaqueous liquid suspension.

The composition may be administered topically, that is by non-systemic administration. This includes the application externally to the epidermis or the buccal cavity and the instillation of such a compound into the ear, eye and nose, such that compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Lotions according to the present invention include those suitable for application to the skin or eye which are suitable, for example, for use in UV protection. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

The composition in accordance with the present invention may be administered parenterally, that is by intravenous, intramuscular, subcutaneous intranasal, intrarectal, intravaginal or intraperitoneal administration. The subcutaneous and intramuscular forms of parenteral administration are generally preferred. Appropriate dosage forms for such administration may be prepared by conventional techniques. The composition may also be administered by inhalation, that is by intranasal and oral inhalation administration. Appropriate dosage forms for such administration, such as an aerosol formulation or a metered dose inhaler, may be prepared by conventional techniques.

In a different preferred embodiment of the composition of the present invention said composition is a diagnostic composition optionally further comprising suitable means for detections.

Said means for detection comprise, for example, (a) chromophore(s), (a) fluorexcent dye(s), (a) radionucleotide(s), biotin or DIG. These labeling means may be coupled to nucleotide analogues. Labeling of amplified cDNA can be performed as described in the appended examples or as described, inter alia, in Spirin (1999), Invest. Opthamol. Vis. Sci. 40, 3108-3115.

The present invention also relates to a use of the multifunctional polypeptide of the present invention, the polynucleotide of the present invention or the vector of the present invention for the preparation of a pharmaceutical composition for the treatment of cancer, infections and/or autoimmune conditions, cancer, i.e. maligned (solid) tumors and hematopoietic cancer forms (leukemias and lymphomas), benigne tumors such as benigne hyperplasia of the prostate gland (BPH), autonomous adenomes of the thyroid gland or of other endocrine glands or adenomas of the colon; initial stages of the malignancies, infectious diseases, caused by viruses, bacteria, fungi, protozoa or helmints, auto immune diseases wherein the elimination of the subpopulation of immune cells is desired that causes the disease; prevention of transplant rejection or allergies.

In a preferred embodiment of the use of the present invention said infection is said infection is a viral, a bacterial or a fungal infection, wherein said cancer is a head and neck cancer, gastric cancer, oesaphagus cancer, stomach cancer, colorectal cancer, coloncarcinoma, cancer of liver and intrahepatic bile ducts, pancreatic cancer, lung cancer, small cell lung cancer, cancer of the larynx, breast cancer, mamma carcinoma, malignant melanoma, multiple myeloma, sarcomas, rhabdomyosarcoma, lymphomas, folicular non-Hodgkin-lymphoma, leukemias, T- and B-cell-leukemias, Hodgkin-lymphoma, B-cell lymphoma, ovarian cancer, cancer of the uterus, cervical cancer, prostate cancer, genital cancer, renal cancer, cancer of the testis, thyroid cancer, bladder cancer, plasmacytoma or brain cancer or wherein said autoimmune condition is ankylosing spondylitis, acute anterior uveitis, Goodpasture's syndrome, Multiple sclerosis, Graves' disease, Myasthenia gravis, Systemic lupus erythematosius, Insulin-dependent diabetes mellitus, Rheumatoid arthritis, Pemphigus vulgaris, Hashimoto's thyroiditis or autoimmune Hepatitis.

The present invention also relates to a use of the polynucleotide of the present invention or the vector of the present invention for the preparation of a composition for gene therapy.

It is envisaged by the present invention that the various polynucleotides and vectors encoding the above described phosphotonin peptides or polypeptides are administered either alone or in any combination using standard vectors and/or gene delivery systems, and optionally together with a pharmaceutically acceptable carrier or excipient. For example, the polynucleotide of the invention can be used alone or as part of a vector to express the (poly)peptide of the invention in cells, for, e.g., gene therapy or diagnostics of diseases related to disorders referred to above. The polynucleotides or vectors of the invention are introduced into the cells which in turn produce the (poly)peptide. Subsequent to administration, said polynucleotides or vectors may be stably integrated into the genome of the subject. On the other hand, viral vectors may be used which are specific for certain cells or tissues and persist in said cells. Suitable pharmaceutical carriers and excipients are well known in the art. The polynucleotides or vectors prepared according to the invention can be used for the prevention or treatment or delaying of different kinds of the diseases referred to above.

In the above-described embodiments, the vector of the present invention may preferably be a gene transfer or targeting vector. Gene therapy, which is based on introducing therapeutic genes, for example for vaccination into cells by ex-vivo or in-vivo techniques is one of the most important applications of gene transfer. Suitable vectors, methods or gene-delivering systems for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813, Isner, Lancet 348 (1996), 370-374; Muhlhauser, Circ. Res. 77 (1995), 1077-1086; Onodua, Blood 91 (1998), 30-36; Verzelefti, Hum. Gene Ther. 9 (1998), 2243-2251; Verma, Nature 389 (1997), 239-242; Anderson, Nature 392 (Supp. 1998), 25-30; Wang, Gene Therapy 4 (1997), 393-400; Wang, Nature Medicine 2 (1996), 714-716; WO 94/29469; WO 97/00957; U.S. Pat. Nos. 5,580,859; 5,589,466; 4,394,448 or Schaper, Current Opinion in Biotechnology 7 (1996), 635-640, and references cited therein.

The polynucleotides and vectors of the invention may be designed for direct introduction or for introduction via liposomes, or viral vectors (e.g. adenoviral, retroviral) into the cell. Preferably, said cell is a germ line cell, embryonic cell, or egg cell or derived therefrom, most preferably said cell used for introduction is a stem cell. As mentioned above, suitable gene delivery systems may include liposomes, receptor-mediated delivery systems, naked DNA, and viral vectors such as herpes viruses, retroviruses, adenoviruses, and adeno-associated viruses, among others. Delivery of nucleic acids to a specific site in the body for gene therapy may also be accomplished using a biolistic delivery system, such as that described by Williams (Proc. Natl. Acad. Sci. USA 88 (1991), 2726-2729).

It is to be understood that the introduced polynucleotides and vectors express the gene product after introduction into said cell and preferably remain in this status during the lifetime of said cell. For example, cell lines which stably express the polynucleotide under the control of appropriate regulatory sequences may be engineered according to methods well known to those skilled in the art. Rather than using expression vectors which contain viral origins of replication, host cells can be transformed with the polynucleotide of the invention and a selectable marker, either on the same or separate plasmids. Following the introduction of foreign DNA, engineered cells may be allowed to grow for 1-2 days in an enriched media, and then are switched to a selective media. The selectable marker in the recombinant plasmid confers resistance to the selection and allows for the selection of cells having stably integrated the plasmid into their chromosomes and grow to form foci which in turn can be cloned and expanded into cell lines. Such engineered cell lines are also particularly useful in screening methods for the detection of compounds involved in, e.g., activation or stimulation of phosphate uptake.

A number of selection systems may be used, including but not limited to the herpes simplex virus thymidine kinase (Wigler, Cell 11 (1977), 223), hypoxanthine-guanine phosphoribosyltransferase (Szybalska, Proc. NatI. Acad. Sci. USA 48 (1962), 2026), and adenine phosphoribosyltransferase (Lowy, Cell 22 (1980), 817) in tk⁻, hgprt⁻ or aprt⁻ cells, respectively. Also, antimetabolite resistance can be used as the basis of selection for dhfr, which confers resistance to methotrexate (Wigler, Proc. Natl. Acad. Sci. USA 77 (1980), 3567; O'Hare, Proc. Natl. Acad. Sci. USA 78 (1981), 1527), gpt, which confers resistance to mycophenolic acid (Mulligan, Proc. Natl. Acad. Sci. USA 78 (1981), 2072); neo, which confers resistance to the aminoglycoside G-418 (Colberre-Garapin, J. Mol. Biol. 150 (1981), 1); hygro, which confers resistance to hygromycin (Santerre, Gene 30 (1984), 147); or puromycin (pat, puromycin N-acetyl transferase). Additional selectable genes have been described, for example, trpB, which allows cells to utilize indole in place of tryptophan; hisD, which allows cells to utilize histinol in place of histidine (Hartman, Proc. Natl. Acad. Sci. USA 85 (1988), 8047); and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.).

The invention further relates to a method for the treatment of cancer, infections or autoimmune conditions comprising introducing the polypeptide of the present invention, the polynucleotide of the present invention or the vector of the present invention or the composition of the present invention into a mammal affected by said malignancies or diseases.

Suitable routes and doses of administration etc. have been discussed in connection with the pharmaceutical composition of the invention herein above.

Furthermore, the present invention relates to a method for delaying a pathological condition comprising introducing the polypeptide of the present invention, the polynucleotide of the invention or the vector of the present invention or the composition of the present invention into a mammal affected by said pathological condition.

In a preferred embodiment of one method of the present invention said mammal is a human.

Finally, the invention relates to a kit comprising the multifunctional polypeptide of the invention, the polynucleotide of the present invention, the vector of the present invention, the cell of the invention or the composition of the present invention.

The components of the kit or the diagnostic composition of the present invention may be packaged in containers such as vials, optionally in buffers and/or solutions. If appropriate, one or more of said components may be packaged in one and the same container. Additionally or alternatively, one or more of said components may be absorbed to a solid support such as, e.g., a nitrocellulose filter or nylon membrane, or to the well of a microtitre-plate.

The figures show:

FIG. 1 shows the nucleotide and amino acid sequence of soluble NKG2D containing a C-terminal histidine-tag. Restriction sites used for cloning are shown at the beginning (EcoRI) and the end (SalI) of the nucleotide sequence.

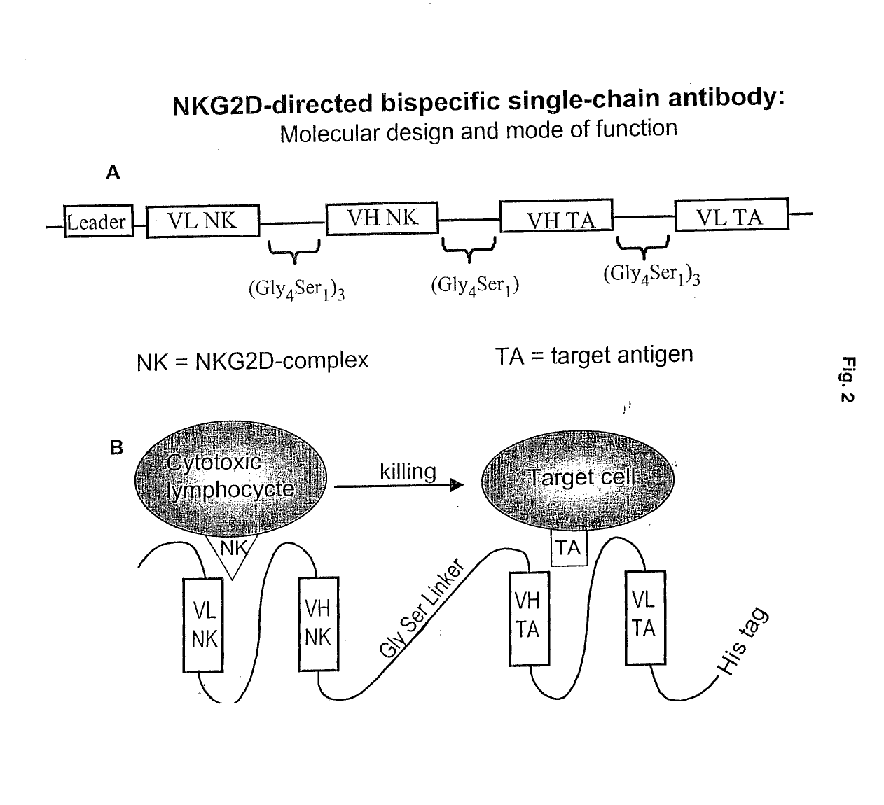

FIG. 2 shows the molecular design of an NKG2D-directed bispecific single-chain antibody at the DNA level (panel A) and the protein level (panel B). The mode of function of the bispecific antibody is also shown in panel B.

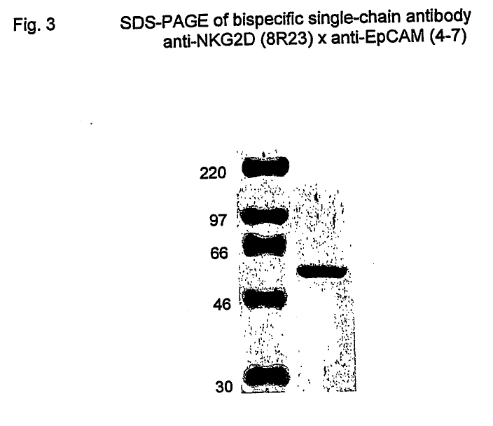

FIG. 3: SDS-PAGE of bispecific single-chain antibody anti-NKG2D (8R23)×anti-EpCAM (4-7) (right lane); the left lane shows a molecular weight marker.

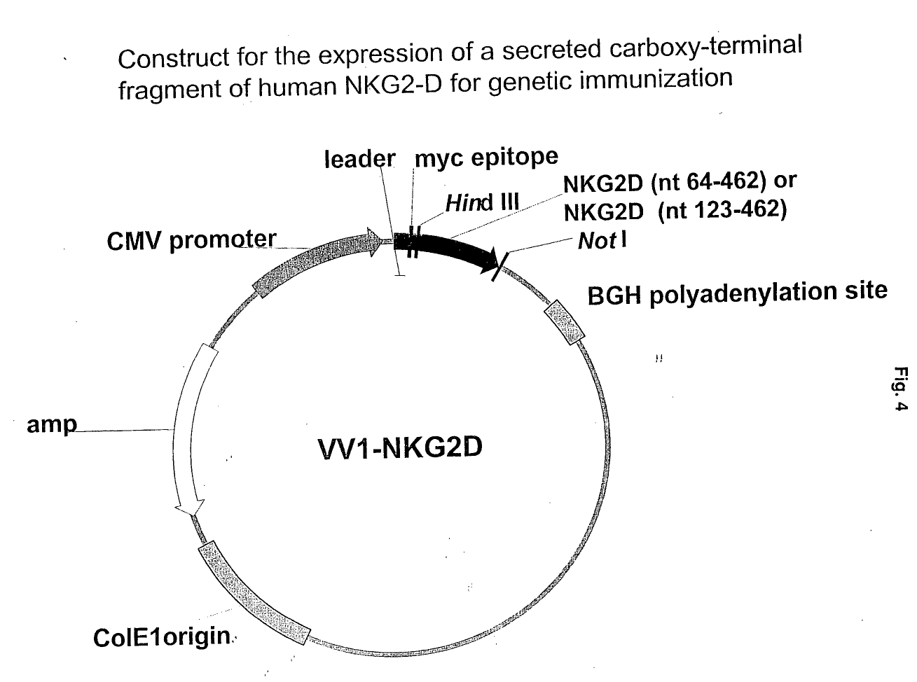

FIG. 4: Expression vector encoding a secreted carboxy-terminal fragment of human NKG2-D used for genetic immunization.

The expression of the NKG2-D fragment from the vector shown is controlled by the immediate-early promoter of the human cytomegalovirus (CMV). The NKG2-D fragment consists of a leader peptide which is derived from the murine immunoglobulin kappa light chain, followed by a human myc epitope. The coding sequence of NKG2-D is terminated by its cognate stop codon. BGH polyadenylation site, bovine growth hormone polyadenylation site; amp, ampicillin resistance gene; ColE1 origin, ColE1 origin of replication.

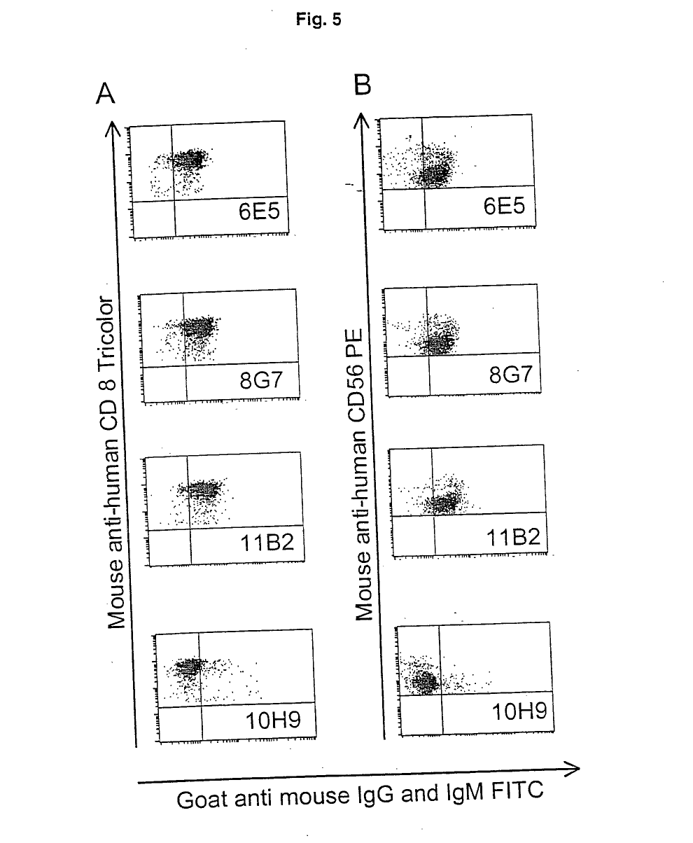

FIG. 5: Selection of hybridomas specifically binding to NKG2-D-positive target cells.

The binding of three distinct monoclonal antibodies in hybridoma supernatants 6E5, 8G7 and 11B2 to either CD8-positive T cells (A) or to CD56-positive natural killer cells is shown by FACS analysis. Abbreviations are 6E5: 6E5/A7, 8G7: 8G7/C10 and 11B2: 11B2/D10.

10H9 is a control with a hybridoma supernatant lacking NKG2-D binding activity. The various detection antibodies are indicated in the Figure.

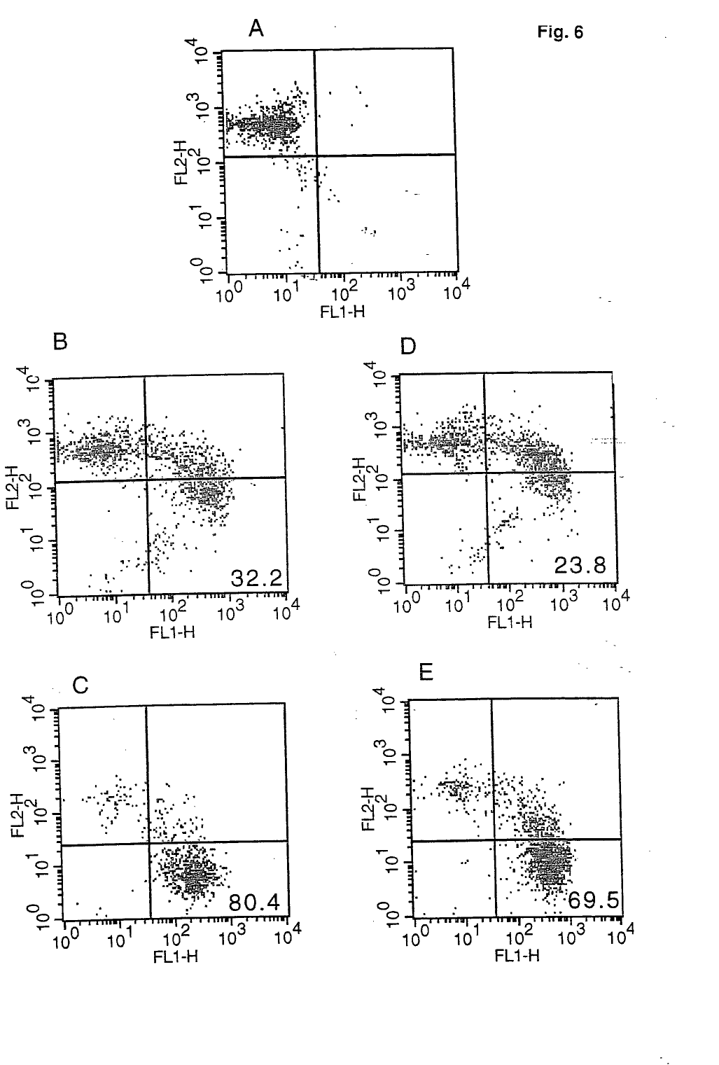

FIG. 6: Enhancing effect of a monoclonal antibody directed against NKG2-D on priming of naive T cells.

Native T cells expressing the marker CD45RA (A) are found in FACS scans in the upper left gate. Naive T cells were primed in the presence of an EpCAM-expressing target cell line (EpCAM/17-1A-transfected CHO cells) by a combination of a B7-1×anti-EpCAM fusion protein and a single chain bispecific anti-EpCAM×anti-CD3 molecule (B-E) in the absence (D and E) or presence (B and C) of a monoclonal antibody against NKG2-D called BAT221. Primed T cells expressing the marker CD45 RO appear in the lower right gate. Numbers-give the percentage of primed, previously native T cells. Fluorescence 1: FITC-labeled anti-CD45RO; fluoresence 2: phycoerythrin-conjugated anti-CD45RA.

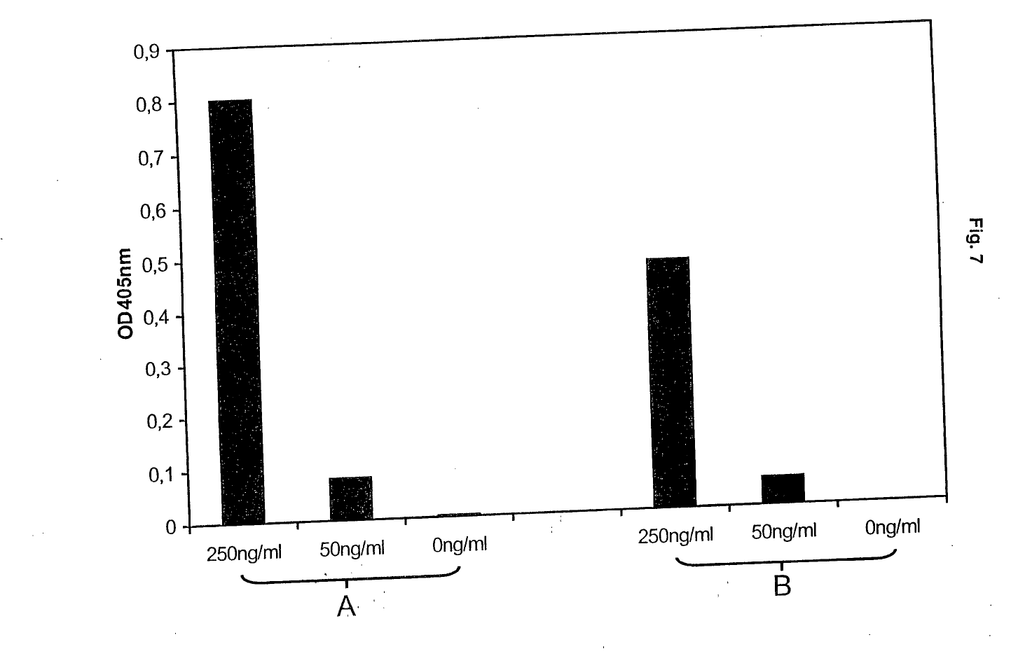

FIG. 7: Enhancing effect of a monoclonal antibody directed against NKG2-D on TNF production by T cells.

Naïve T cells were primed in the presence of an EpCAM-expressing target cell line (EpCAM/17-1A-transfected CHO cells) by a combination of a B7-1×anti-EpCAM fusion protein and increasing concentrations, as indicated, of a single chain bispecific anti-EpCAM×anti-CD3 molecule. TNF production was measured by a commercial TNF-α: ELISA in the presence (A) and absence (B) of a monoclonal antibody against NKG2-D, called BAT221.

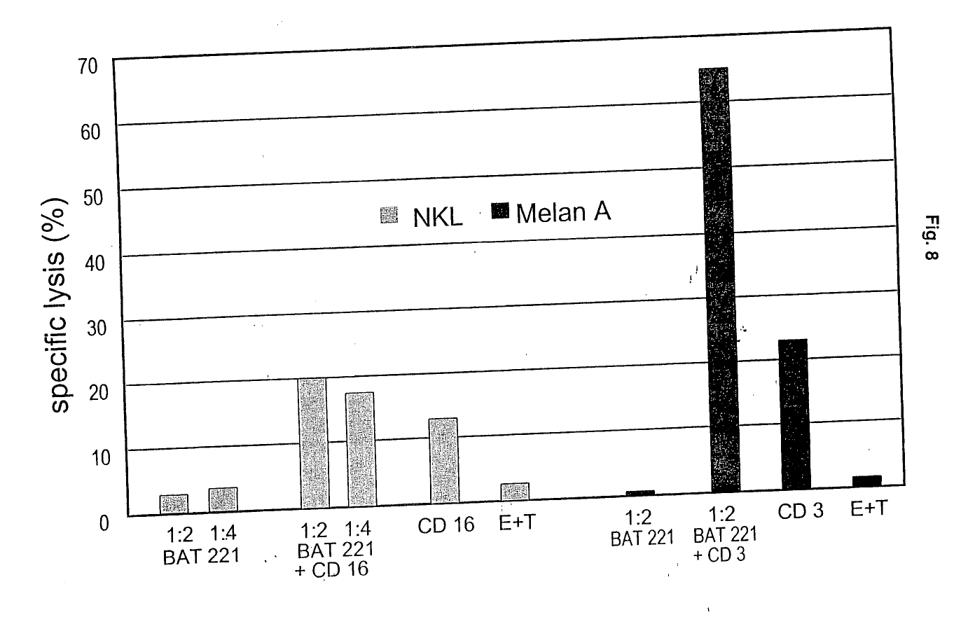

FIG. 8: Cytotoxic activity of Melan A cells and NKL cells redirected against P815 cells by several dilutions of the supernatant of the NKG2D hybridoma BAT 221 in combination with the monoclonal antibodies CD16 (5 μg/ml) and CD3 (0,2 μg/ml) respectively. 200.000 NKL cells or 50000 Melan A cells were added to 10.000 Chromium-51 labeled Kato III cells in the presence of the diluted antibody in a total volume of 200 μl. The backround control (E+T) contains effector cells and target cells without an antibody dilution. The microtiterplates were incubated for 4 h at 37° C., 5% CO₂. After the incubation period 50 μl supernatant were removed from each well and assayed for released ⁵¹Cr in a gamma counter.

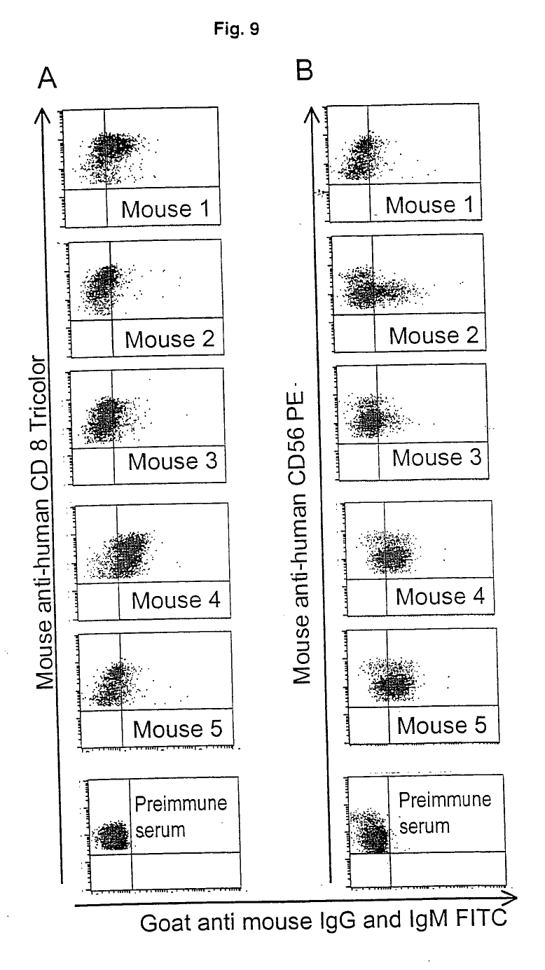

FIG. 9: Detection of a specific immune response in mice immunized with an expression vector encoding a secreted C-terminal fragment of human NKG2-D.

Flowcytometric analysis of the binding activity of a 1:30 serum diliution of five immunized mice to human CD8⁺ T lymphocytes and human NK cells. 200.000 mononucleated cells from peripheral blood of a healthy donors were incubated with diluted serum of the five mice. Bound murine antibody was detected by a fluoresceine (FITC)-conjugated goat-anti-rat Ig (IgG+IgM) antibody diluted 1:100 in PBS. Triple color fluorescence analysis was carried out by applying a positive gate for CD8⁺ (Tricolor) and a negative gate for CD16⁺ (PE) cells thus allowing the detection of FITC-mediated fluorescence exclusively attributed to CD8⁺-T-lymphocytes (phenotype: CD8⁺, CD16⁻) without any contaminating signals from CD8⁺-NK-cells. Similarly, triple color fluorescence analysis was carried out by applying a positive gate for CD56⁺-(PE) and a negative gate for CD3⁺-cells (tricolor) thus allowing the detection of FITC-mediated fluorescence exclusively attributed to NK-cells (phenotype: CD56⁺, CD3⁻) without any contaminating signals from CD56⁺-T-lymphocytes. As negative control a representative serum of an unimmunized mouse was used (preimmune serum). Cells were analyzed by flowcytometry on a FACSscan (Becton Dickinson).

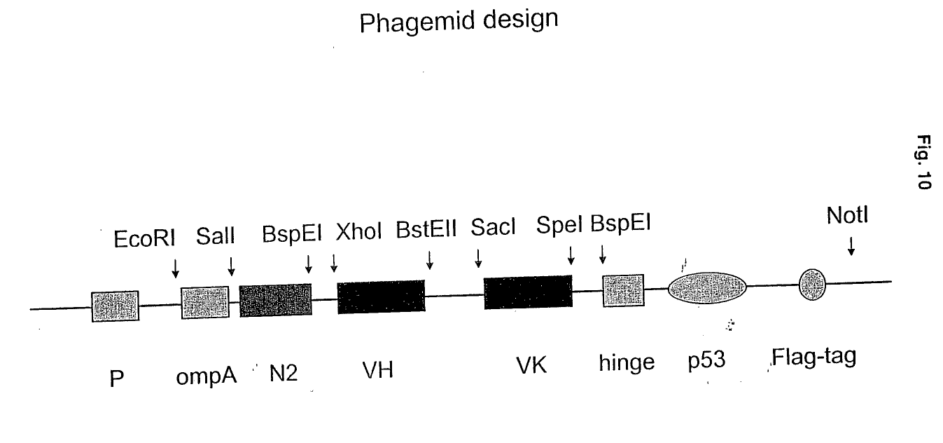

FIG. 10: Design of the phagemid used for expression of N-terminally blocked single chain antibodies in the periplasm of E. coli.

P, bacterial promoter; ompA, leader sequence for periplasmic transport; N2, surrogate N-terminal blocking domain; VH, variable heavy chain domain of scFv; VL, variable light chain domain of scFv; p53, tetramerization domain of transcription factor p53; Flag-tag; influenza virus epitope tag. The positions of various restriction enzyme sites are indicated on top. Essential coding sequences are shown as black boxes.

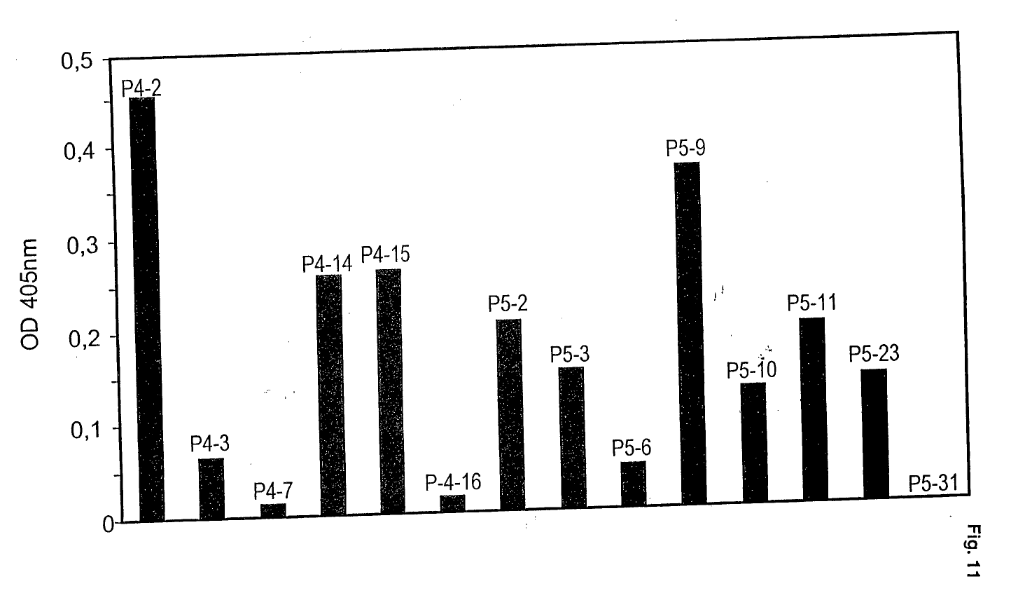

FIG. 11: Detection of NKG2-D-specific, N-terminally blocked single chain Fv fragments produced in the periplasm of E. coli.

In order to increase sensitivity, the binding avidity of single chain Fv antibodies was increased by fusing the tetramerization domain of the transcription factor p53 to the carboxy terminus of N-terminally blocked scFvs. Tetramerized scFvs were detected in periplamsic fractions by ELISA with soluble, recombinant NKG2-D as capture and peroxidase-conjugated anti-FLAG antibody for detection. ELISA signals of various clones are depicted. All clones with signals>0.05 were analyzed further.

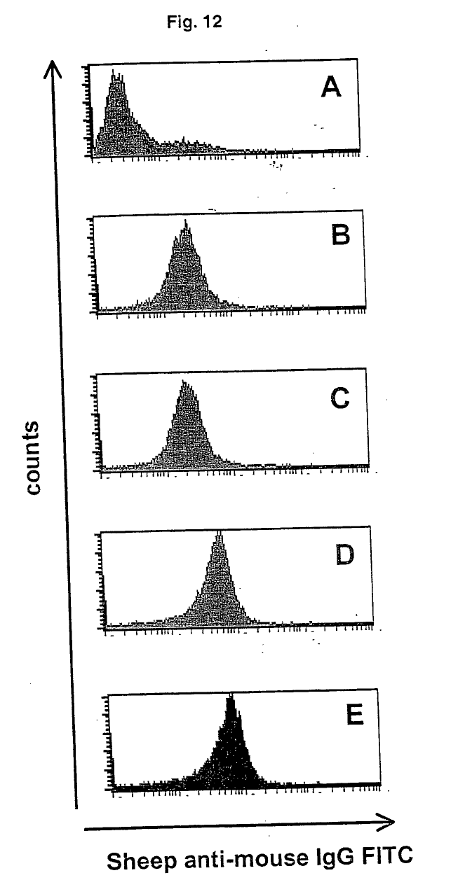

FIG. 12: Transient expression and EpCAM binding of four bispecific molecules targeting NKG2-D.

CHO/dhfr− cells were transiently transfected with expression vectors encoding four different single chain bispecific molecules. In A, a beta-galactosidase gene was transfected as negative control. The various bispecific molecules are B, 3B10×P4-3; C, 3B10×P4-14, D, 3B10×P5-2 and E, 3B10×P5-23. Cell culture supernatants were harvested after 5 days and tested for the expression of bispecific antibodies by FACS analysis for EpCAM-specific binding to the human gastric carcinoam cell line Kato III. Cell-bound bispecific molecules were detected by an FITC-labeled sheep-anti-mouse antibody. FACS histogram blots are shown.

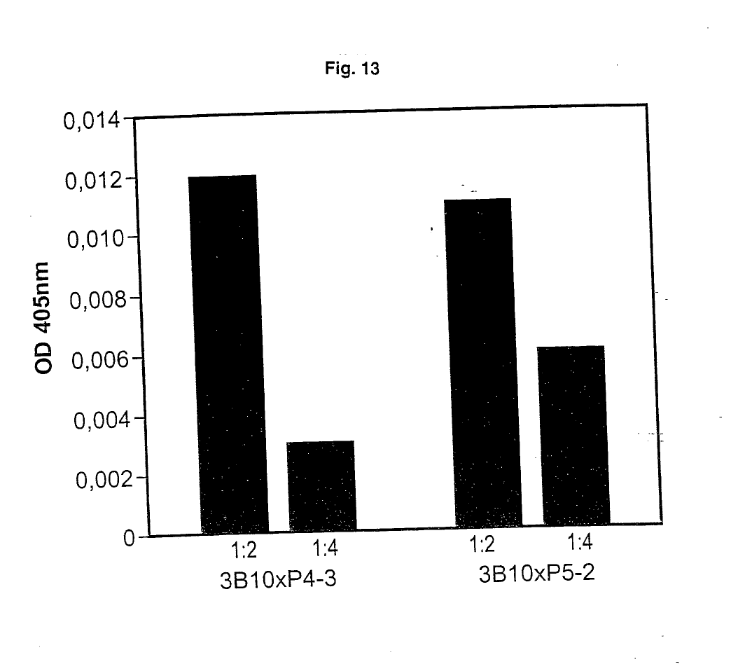

FIG. 13: Characterization of two single chain bispecific antibodies for NKG2-D specific binding in an ELISA.

The two bispecific antibodies 3B10×P4-3 and 3B10×P5-2 were transiently expressed in CHO cell culture supernatants. Binding to coated soluble, recombinant NKG2-D was tested by an ELISA using a peroxidase-conjugated anti-hexahistidine antibody for detection of the hexahistidine-tagged bispecific antibodies. Two different concentrations were tested. A, 1:1 dilution; B, 1:2 dil ution of culture supernatants. As a control, binding of an EpCAM-specific 3B10×anti-CD3 bispecific antibody was used. Values obtained for this non-specific control were subtracted fom the readings shown.

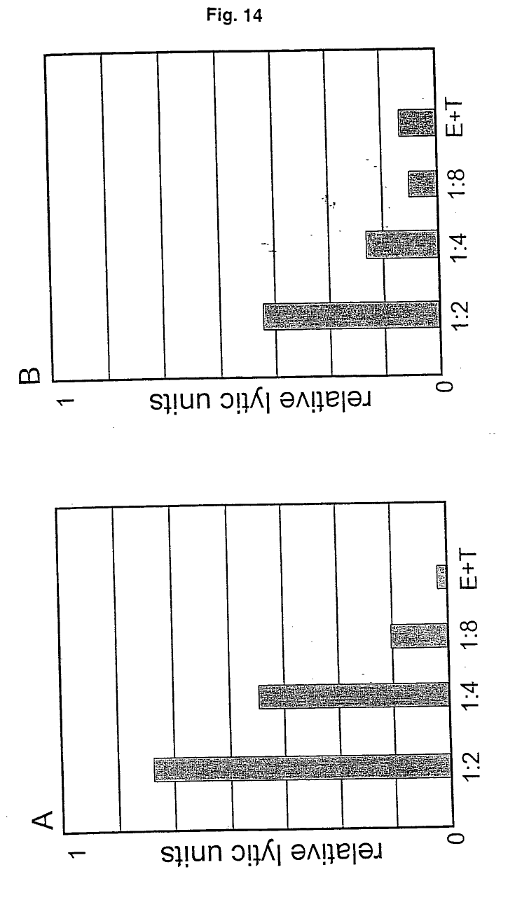

FIG. 14: Cytotoxic activity of Melan A cells (A) and NKL cells (B) redirected against EpCAM-positive Kato cells by the bispecific 3B10×P4-3 antibody. 200.000 NKL cells or 50000 Melan A cells were added to 10.000 Chromium-51 labeled Kato III cells in the presence of serveral dilutions of the bispecific antibody in a total volume of 200 μl. The background control (E+T) contains effector cells and target cells without an antibody dilution. The microtiterplates were incubated for 4 h at 37° C., 5% CO₂. After the incubation period 50 μl supernatant were removed from each well and assayed for released ⁵¹Cr in a gamma counter.

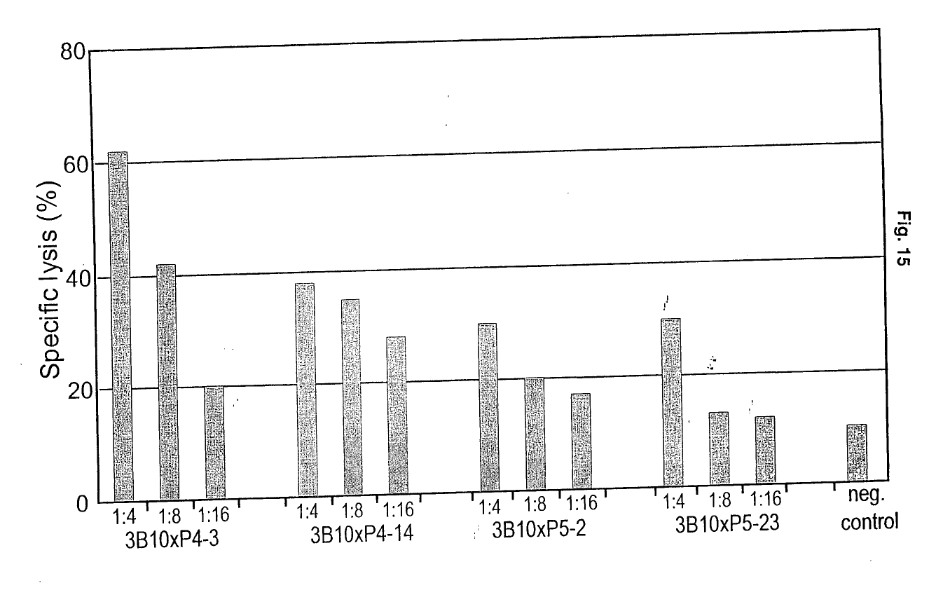

FIG. 15: Specific target cell lysis by four single chain antibodies recruiting peripheral blood mononuclear cells (PBMCs) via NKG2-D.

Four bispecific antibodies all recognizing the EPCAM target on the human gastric carcinoma cell line Kato III by a single chain Fv derived from monoclonal antibody 3B10 were contructed from four distinct scFvs specific for the NK/CD8-specific receptor NKG2-D. Expression vectors encoding the four bispecific antibodies were transfected for transient expression into CHO cells and supernatants collected. Supernatants with secreted bispecific antibodies at the indicated dilutions were tested in cytotox assays for specific lysis of Kato IlIl cells in the presence of human immune effector cells (PBMCs). In the absence of CHO supernatants, no target cell lysis of Kato III cells was observed in the presence of PBMCs. Data shown are the means of triplicate determinations. Cytotoxic activity of PBMC redirected against EPCAM-positive Kato cells by the bispecific antibodies 3B10×P4-3; 3B10×P4-14; 3B10×P5-2 and 3B10×P5-23 in several dilutions. 200.000 PBMCs were added to 10.000 Chromium-51 labeled Kato III cells in the presence of the diluted bispecific antibodies in a total volume of 200 μl. The negative control contains PBMCs and target cells without an antibody dilution. The microtiterplates were incubated for 4 h at 37° C., 5% CO₂. After the incubation period 50 μl supernatant were removed from each well and assayed for released ⁵¹Cr in a gamma counter.

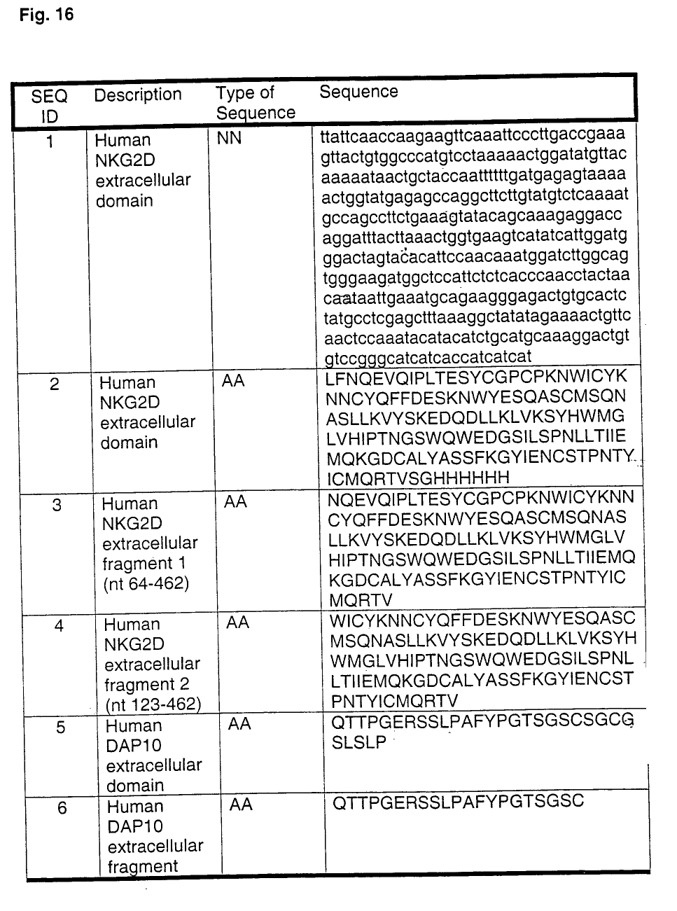

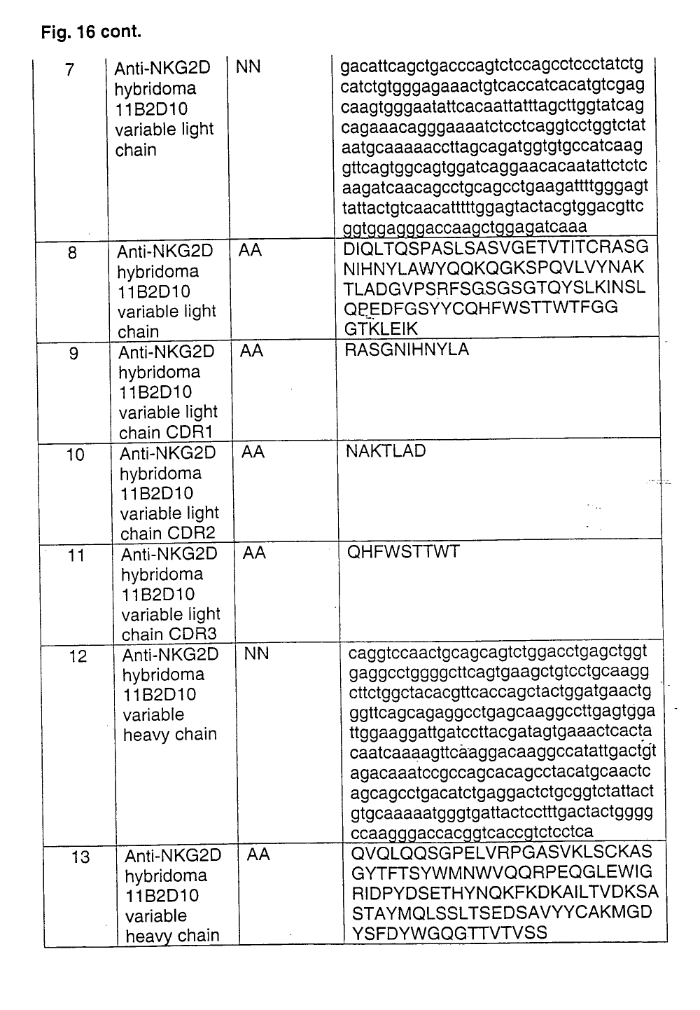

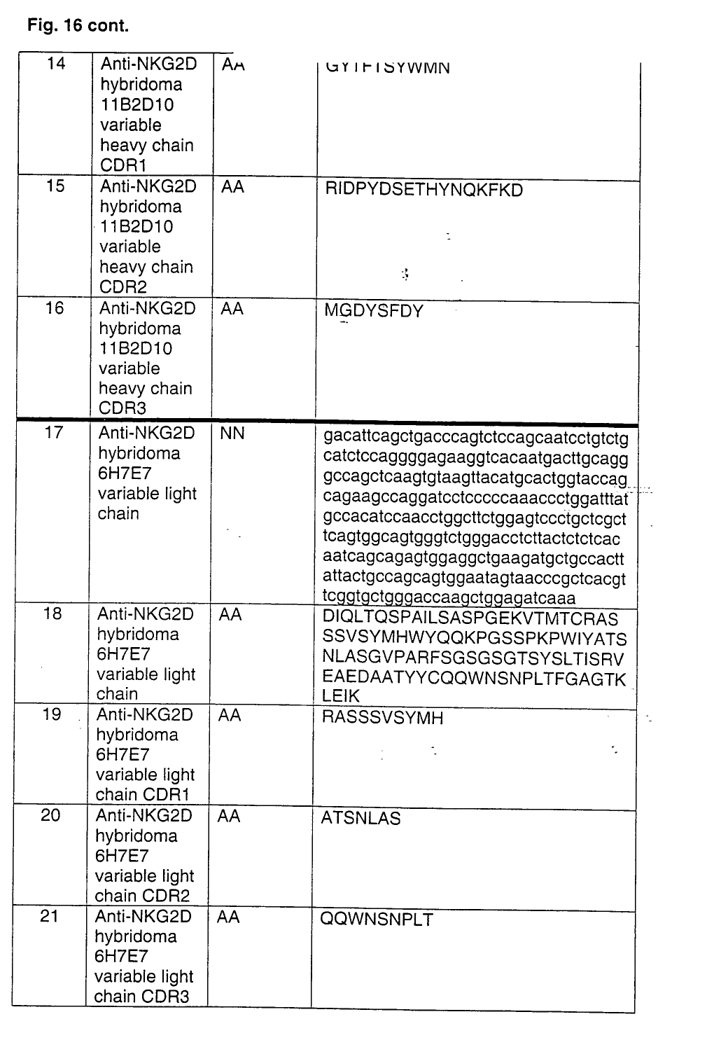

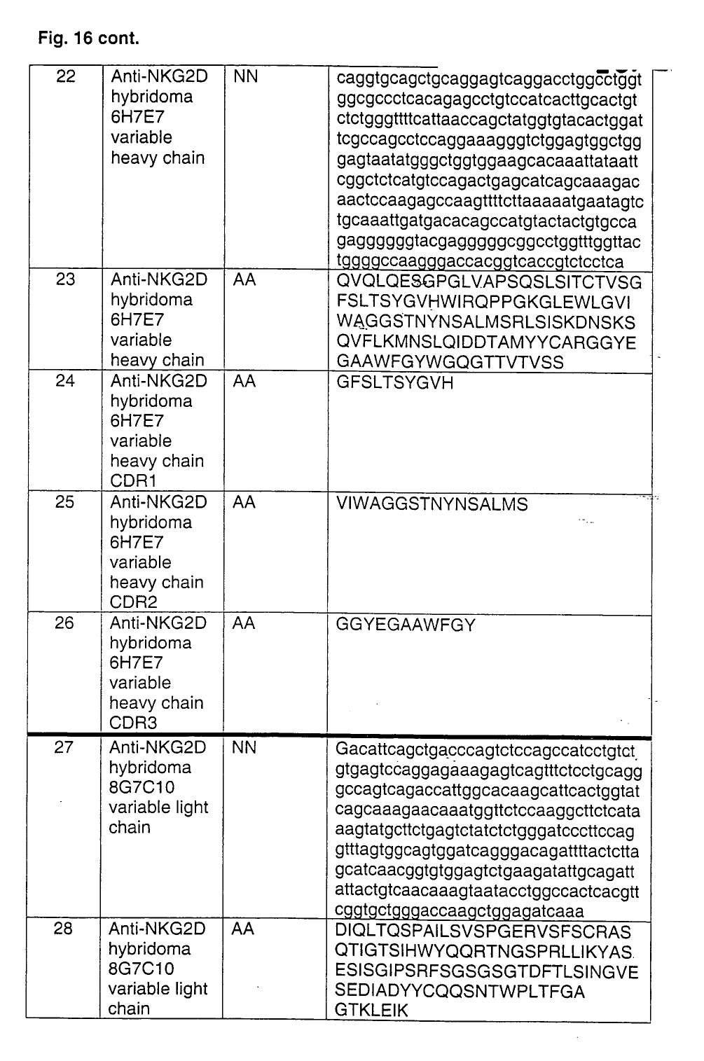

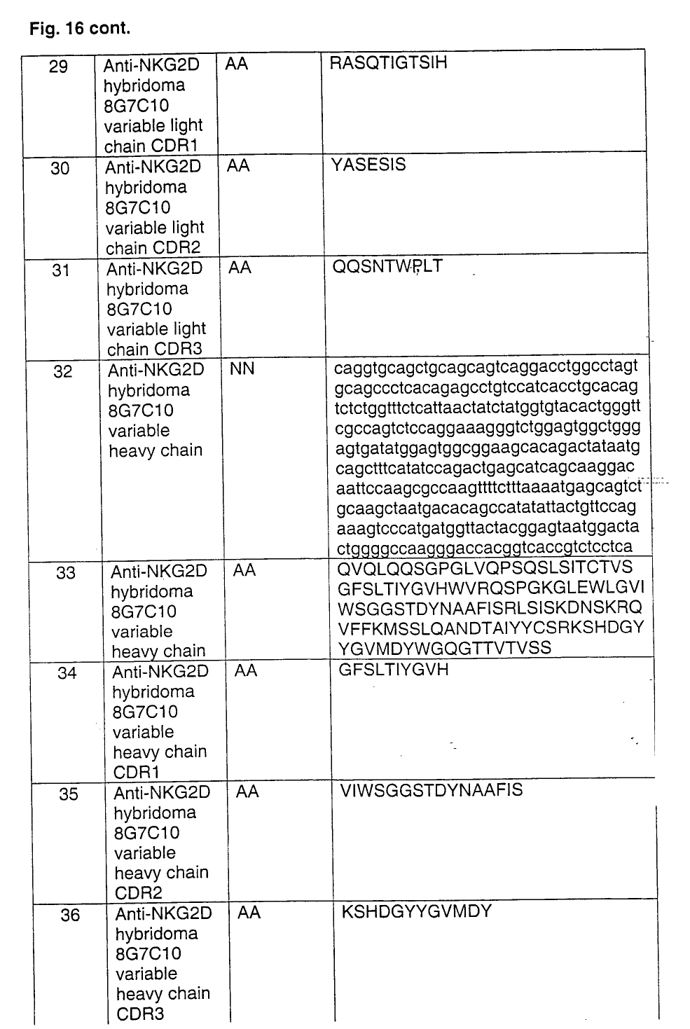

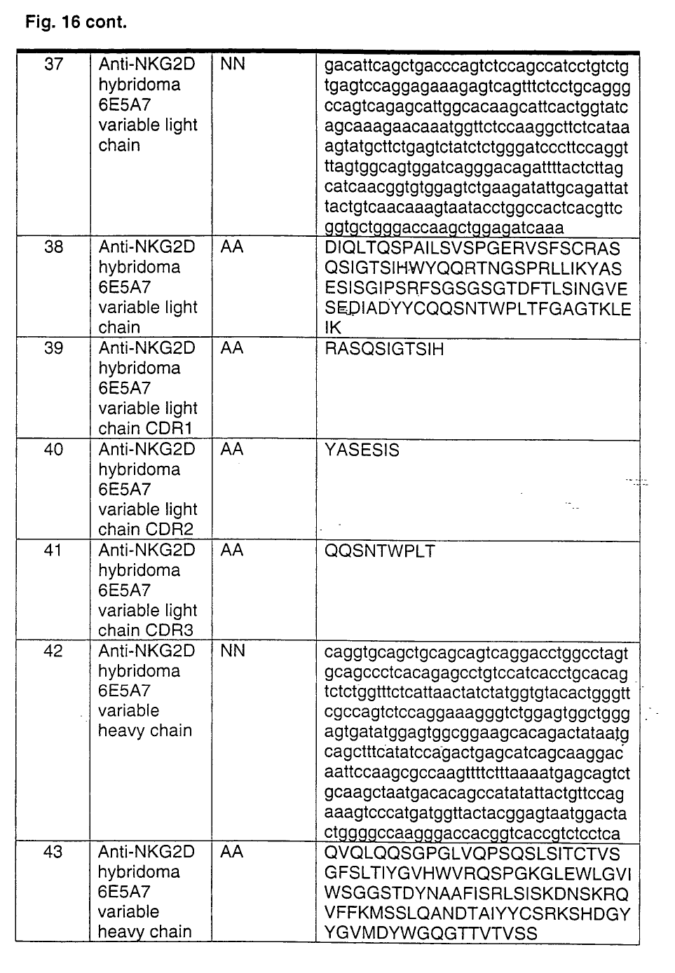

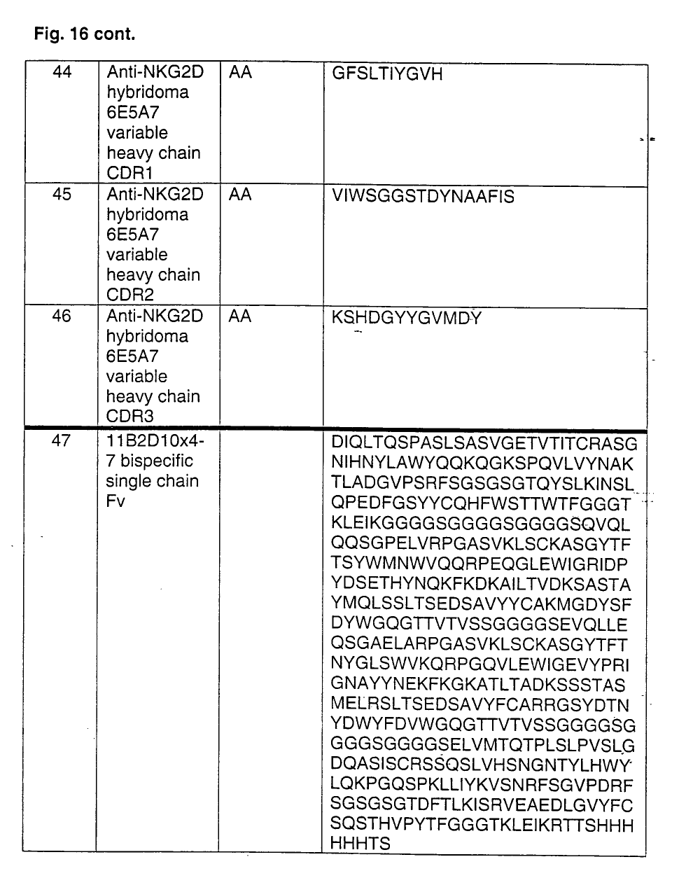

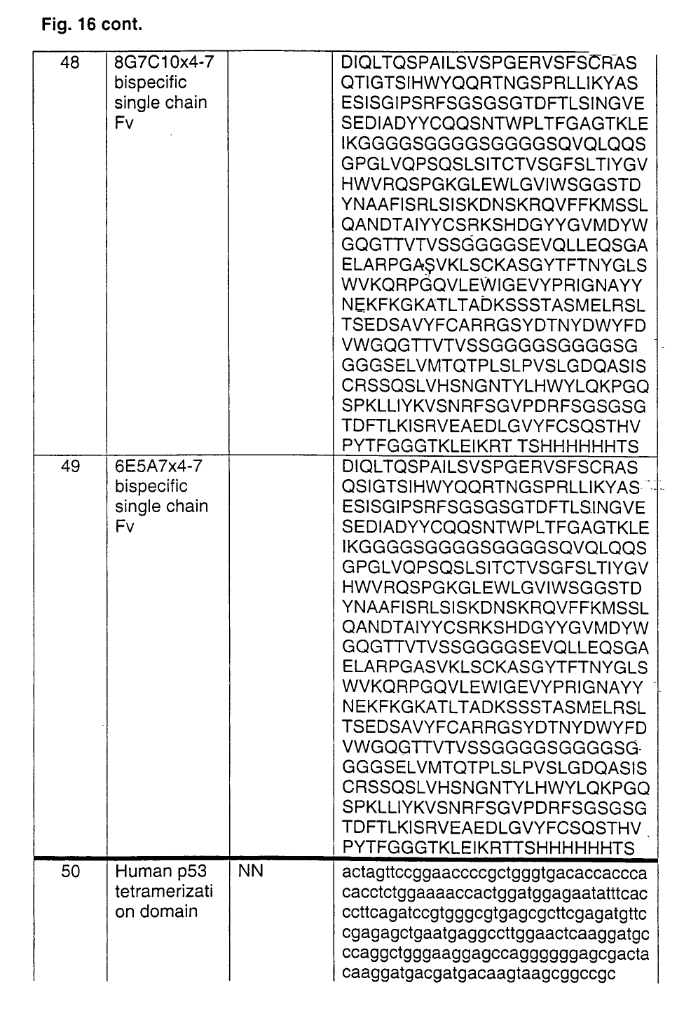

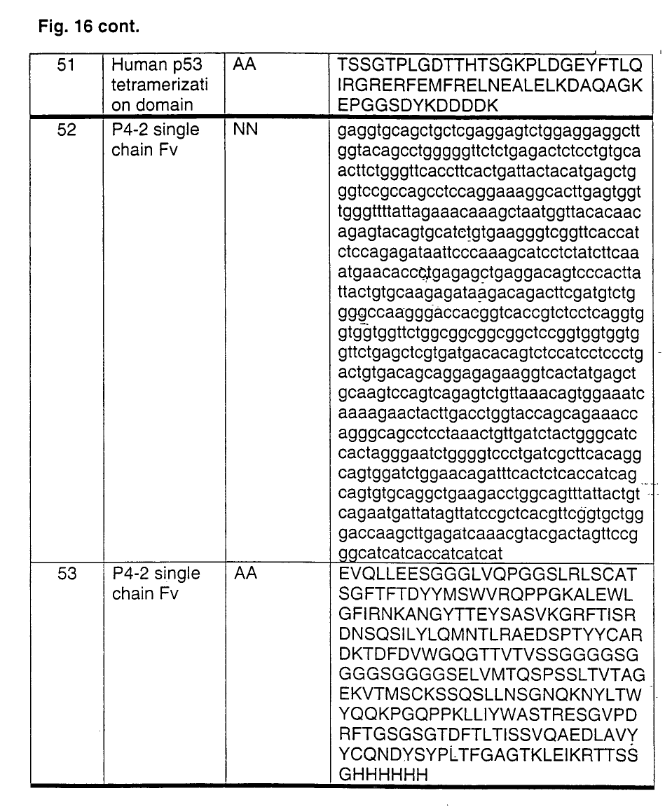

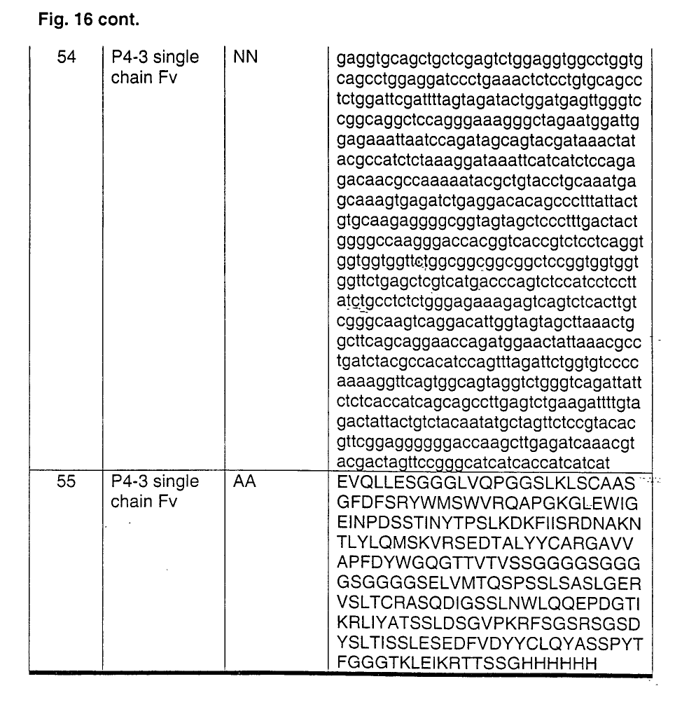

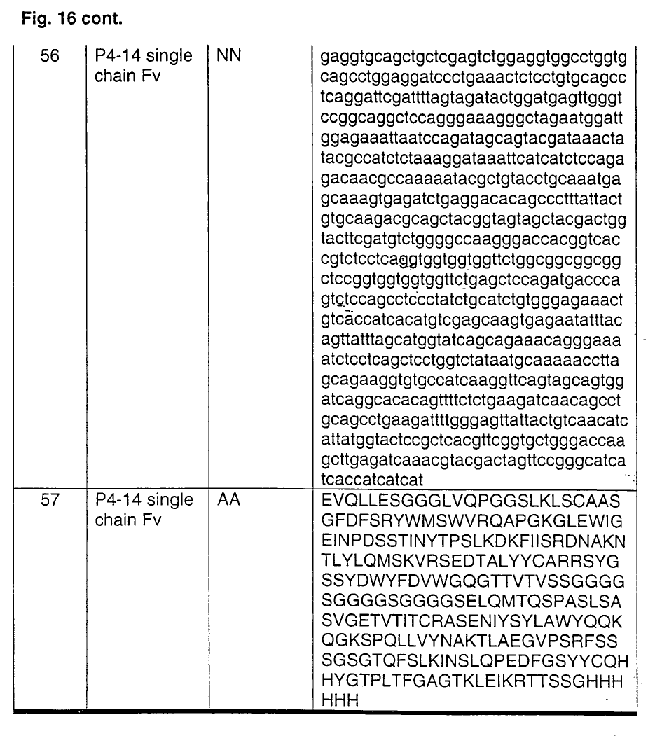

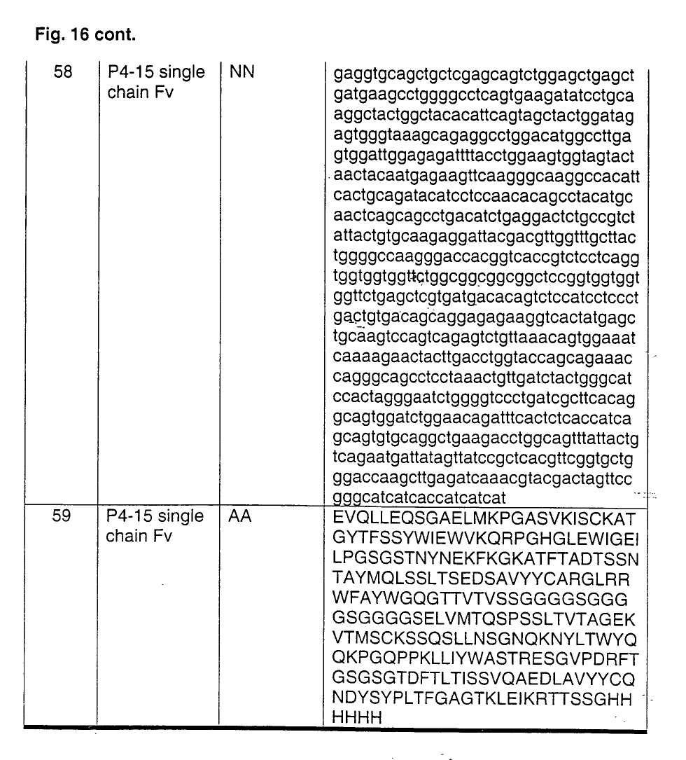

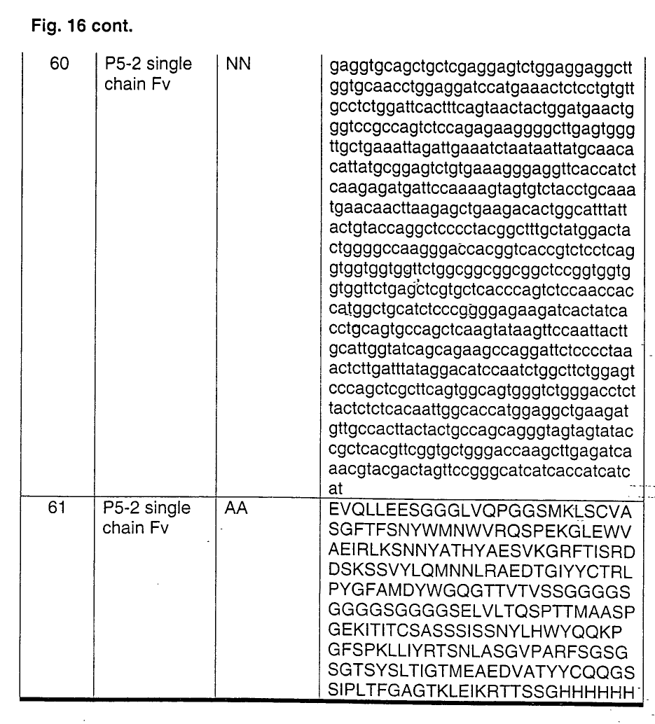

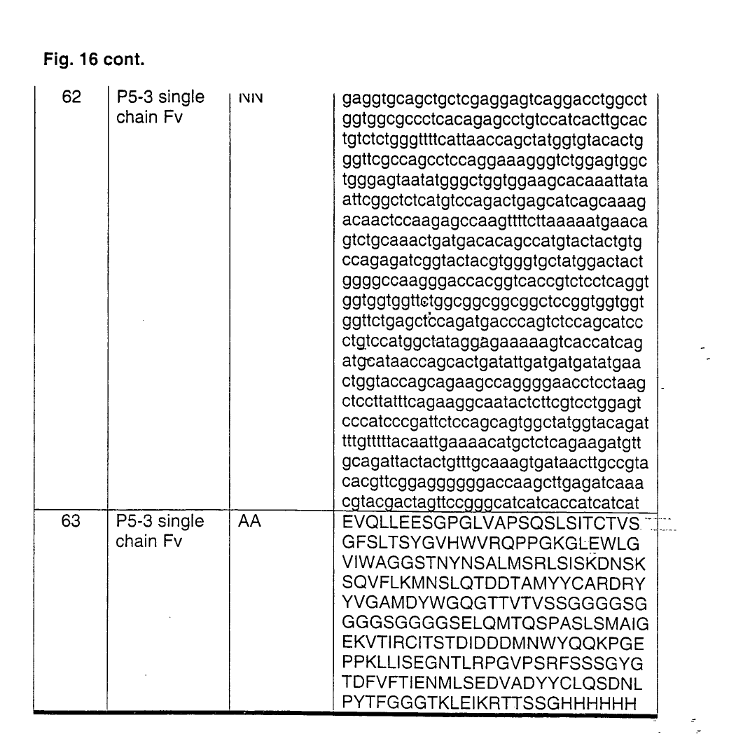

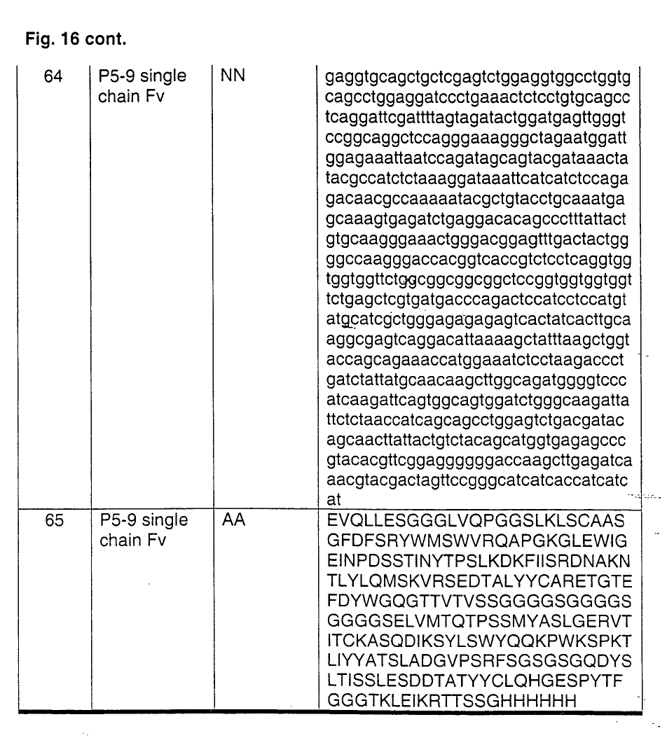

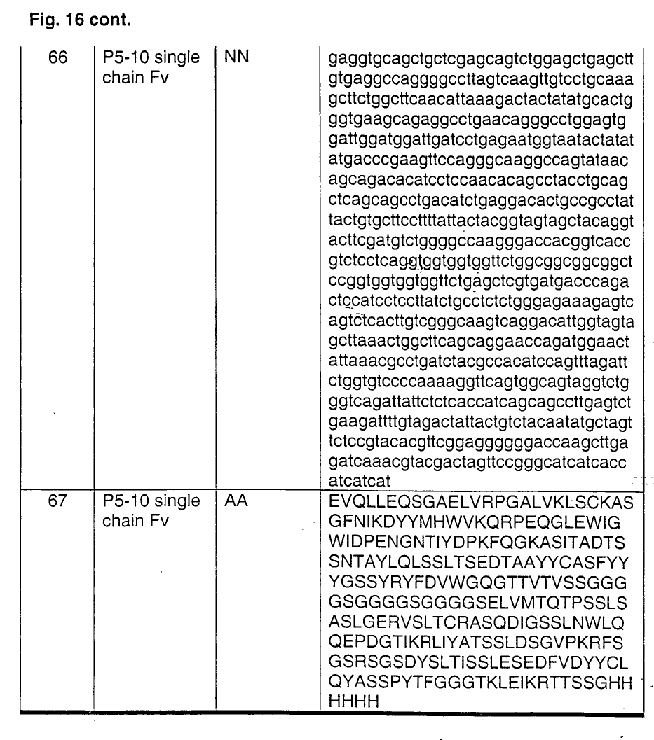

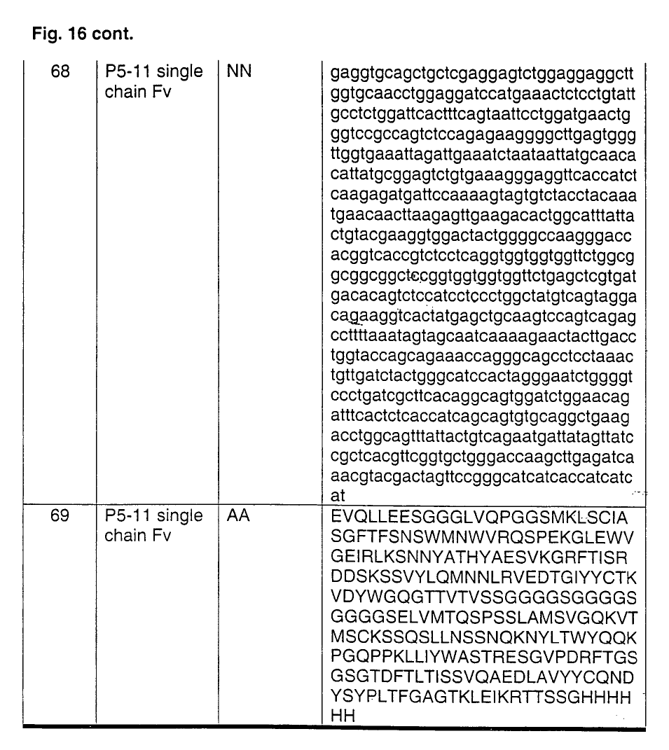

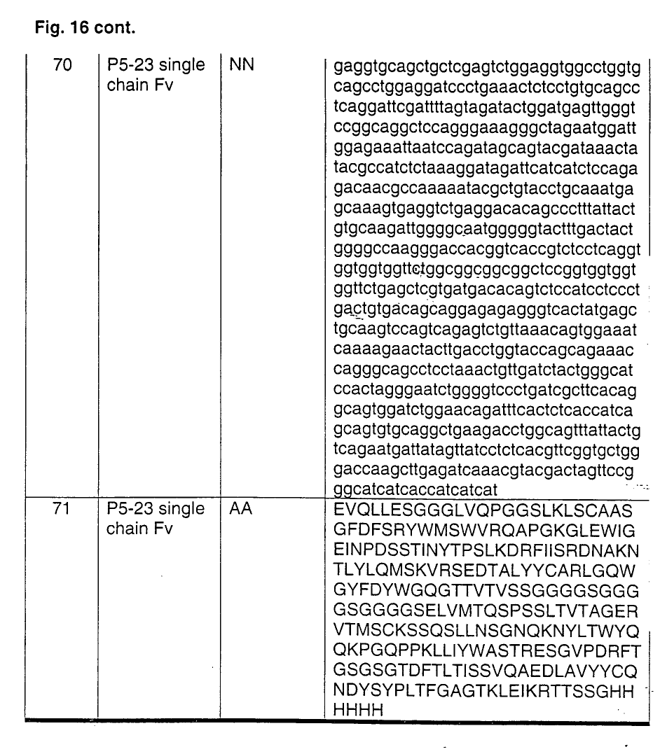

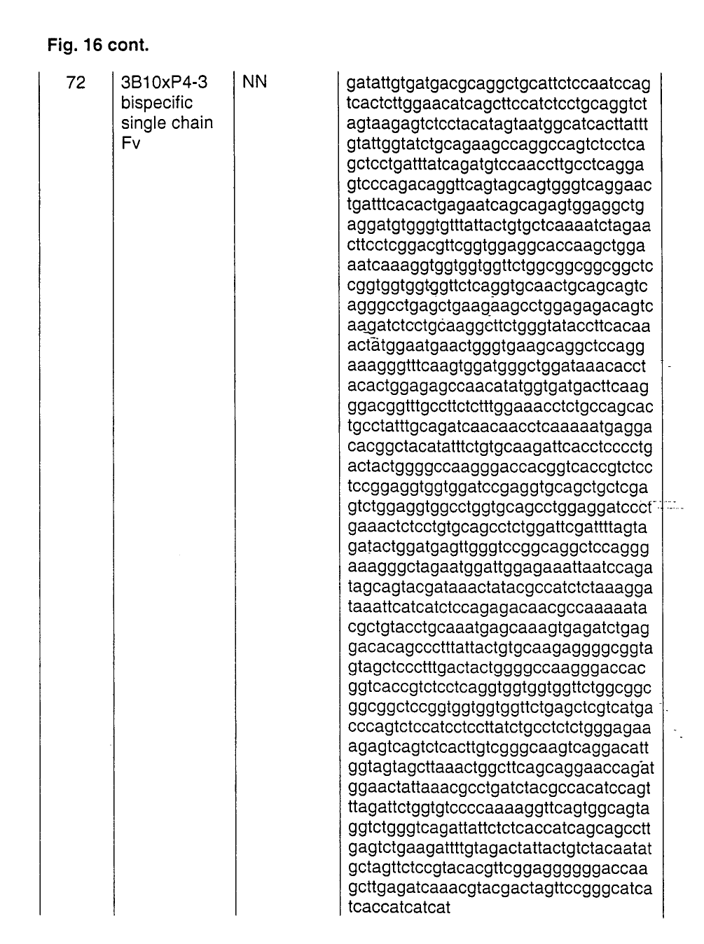

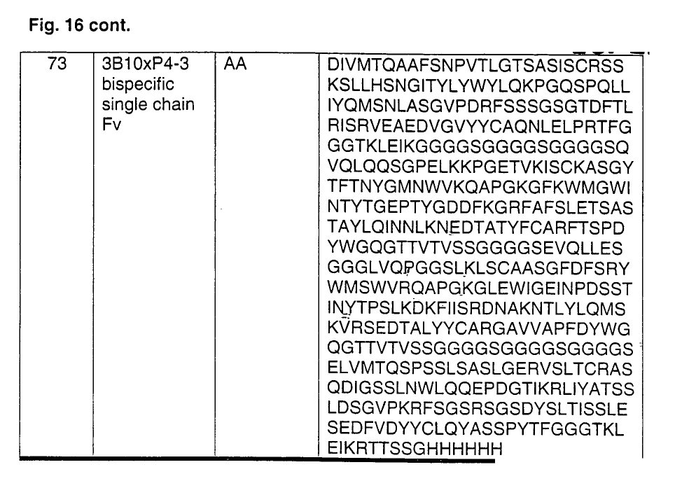

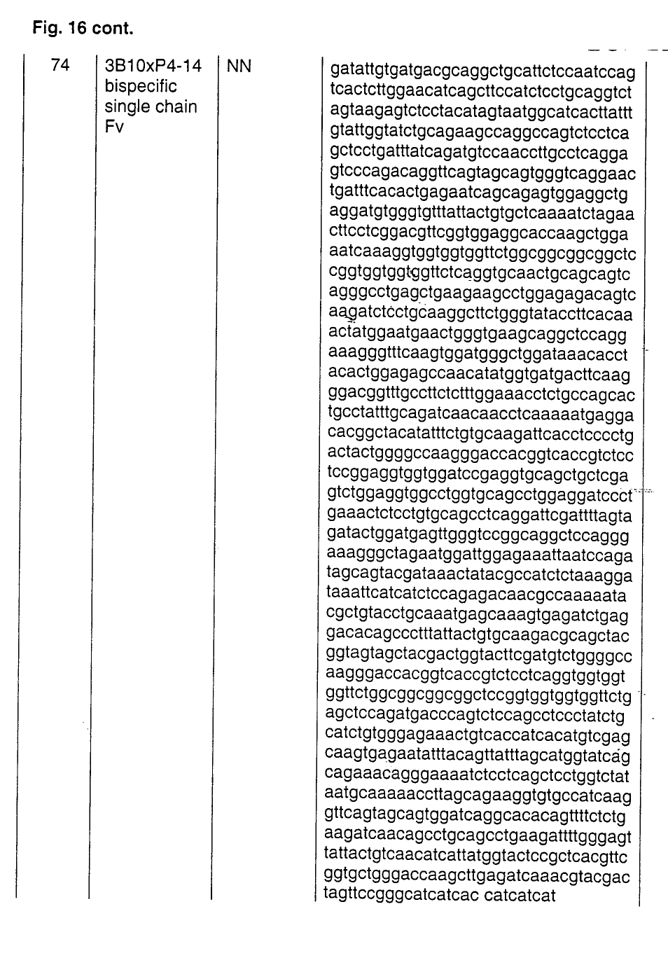

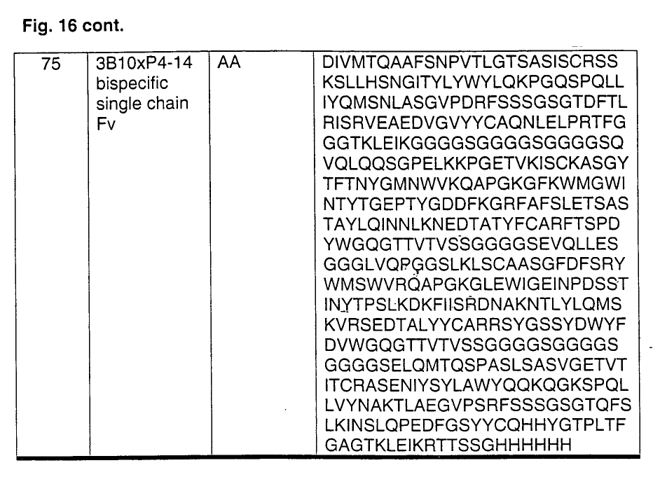

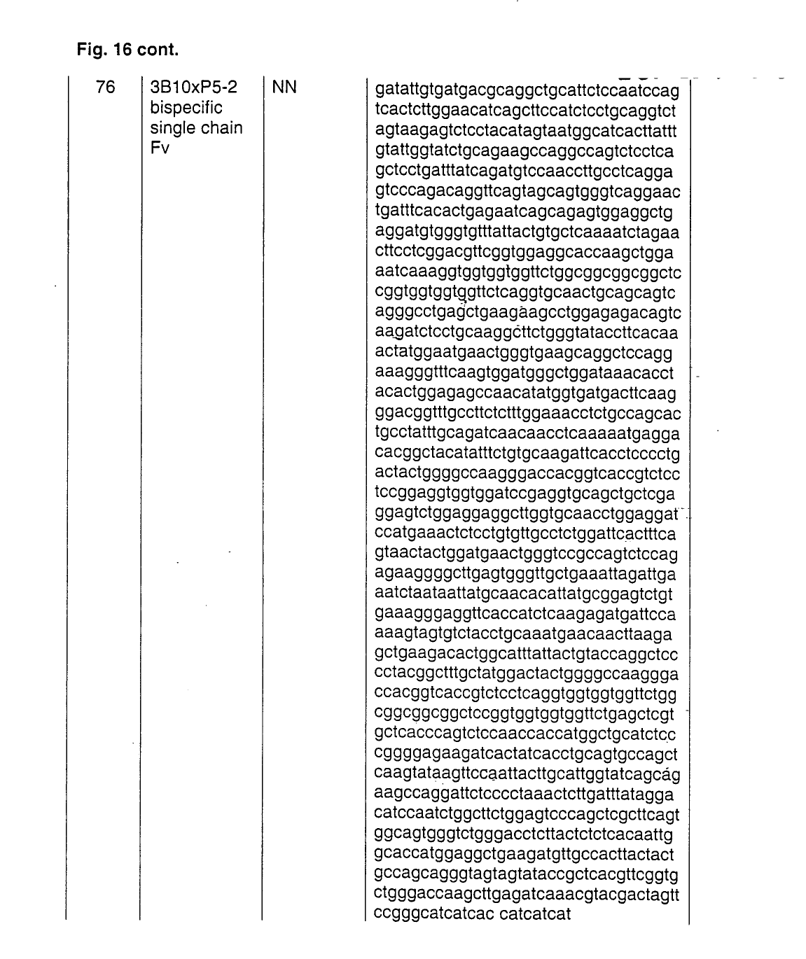

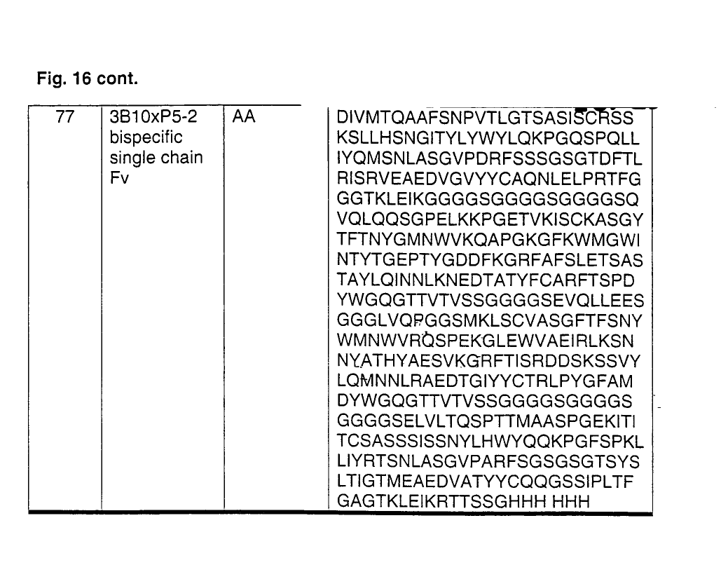

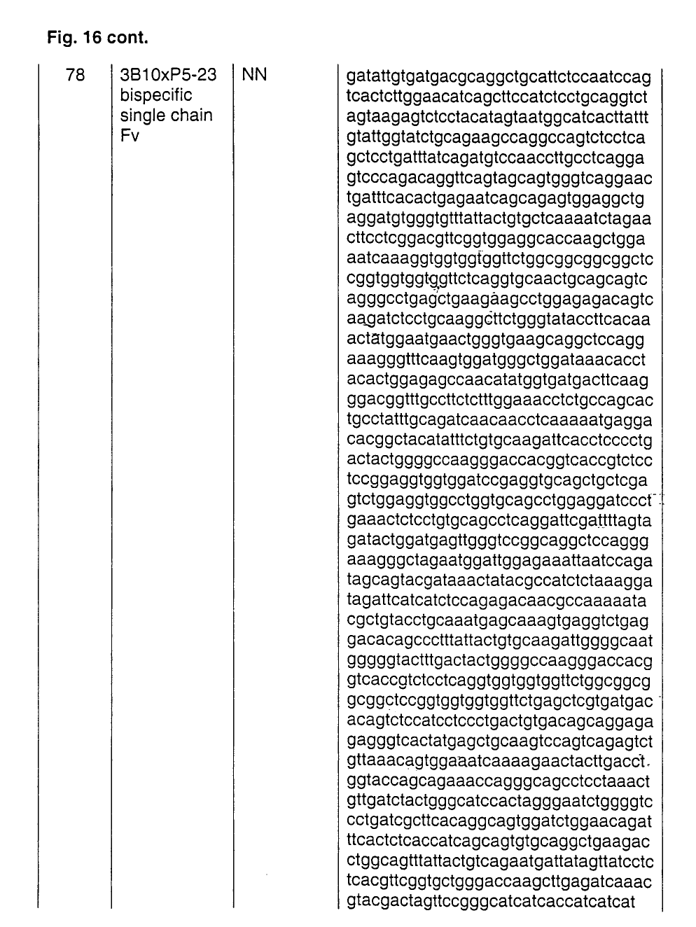

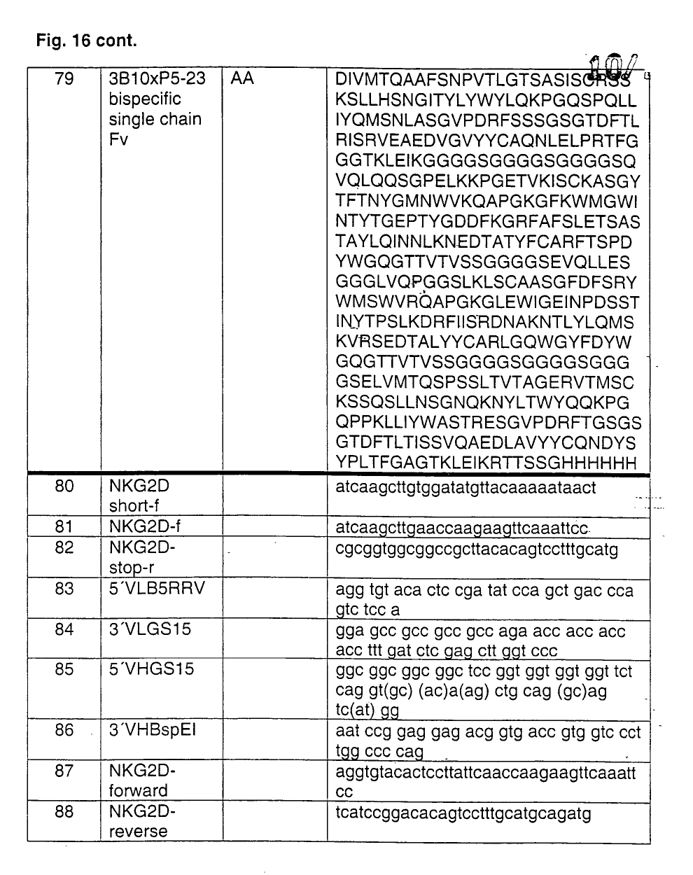

FIG. 16: Compilation of sequences as depicted in the appended examples. The nucleotide sequences are shown in the common 5′-3′ orientation.

The Examples illustrate the invention.

### EXAMPLE 1

Production of Recombinant NKG2D

To obtain the coding DNA-sequence of the extracellular portion of the NKG2D-antigen, cDNA derived from the RNA of peripheral blood mononuclear cells by reverse transcription was used as template for a polymerase chain reaction (PCR).

Total RNA was prepared from peripheral blood mononuclear cells which were separated from a whole-blood sample by ficoll-density centrifugation following standard protocols (J. E. Coligan, Wiley Intersience 1991).

The RNA preparation was performed using a commercially available preparation kit (Quiagen) according to the instructions of the manufacturer.

The cDNA-synthesis was carried out according to standard protocols (Sambrock, Cold Spring Harbor Laboratory Press 1989, second edition)

For the PCR, a pair of primers with the following sequences was used:

Forward primer: 5′-AGGTGTACACTCCTTATTCAACCAAGAAGTTCAAATTCC-3′ (SEQ ID 87)

Reverse primer: 5′-TCATCCGGACACAGTCCTTTGCATGCAGATG-3′ (SEQ ID 88)

In addition to the sequence hybridizing to the NKG2D cDNA-template, the forward primer contains a BsrGI-site and the reverse primer a BspEI-site to allow the cloning of the PCR amplification product.

The product of the PCR-reaction was isolated by means of an agarose-gel electrophoresis, purified using a commercially available kit (Quiagen) according to the instructions of the manufacturer, and then incubated with the restriction enzymes BsrGI and BspEI using standard protocols (Sambrock, Cold Spring Harbor Laboratory Press 1989, second edition). Afterwards a final purification step was performed.

As shown in FIG. 1, the coding sequence of the NKG2D extracellular domain was fused via BsrGI to a murine Ig-heavy chain leader sequence; the BspEI-site was fused with an XmaI-site thus joining the coding sequence of a poly-histidine tag followed by a stop codon (SEQ ID 1 and 2).

The EcoRI/SalI-DNA fragment shown in FIG. 1 consisting of the coding sequences of an N-terminal leader peptide, the NKG2D extracellular domain and a C-terminal histidine-tag, was cloned into the plasmid vector pFastBac1 also prepared by digestion with the restriction enzymes EcoRI and SalI. This plasmid is part of the Bac-to-Bac® Baculovirus expression system (Gibco BRL, instructions of the manufacturer are available at the internet site: http://www2.lifetech.com/catalog/techline/molecular biology/Manuals PPS/bac.pdf. Unless stated otherwise, all procedures related to the Bac-to-Bac® Baculovirus expression system, were carried out according to these instructions).

1 ng DNA of a correct plasmid clone was then tranisformed into DH10Bac competent cells (Bac-to-Bac® expression system). This Escherichia coli strain already carries two other plasmids, (i) a helper plasmid (pMON7124) providing Tn7 transposition functions and (ii) a so-called bacmid (pMON 14272) which is a baculovirus shuttle vector. After transformation of the third plasmid into these cells the coding sequence inserted into pfastBacl is transferred by transposition into the bacmid which contains specific target sites for this transposition. That leads to the destruction of a LacZ-coding sequence which offers the possibility to select colonies with the recombinant bacmid by means of a blue white selection on agar plates containing Bluo-gal, IPTG and a combination of antibiotics according to the instructions of the manufacturer.

White colonies containing the recombinant bacmid with the soluble NKG2D sequence were selected and cultured over night. A specific protocol provided by the manufacturer was used for the preparation of bacmid-DNA from these overnight cultures.

The bacmid-DNA was then used to transfect SF9-insect cells using CellFectin Reagent (Bac-to-Bac® expression system) according to the instructions of the manufacturer. Three days after transfection recombinant baculovirus in the culture supernatant of the transfected cells was harvested. This supernatant is a low titer (approximately 2×10⁷ plaque forming units (pfu) per millilitre) low scale (2 ml) virus stock. (Instructions for insect cell culture, propagation of baculoviruses and protein expression in the baculovirus expression system are available at the internet site: http://www.invitrogen.com/manuals.html. Unless stated otherwise, all procedures related to insect cell culture and protein expression were carried out according to these instructions). For protein expression a high titer and high scale virus stock was required. To obtain such a virus stock the following steps were performed:

Two 25 cm² tissue culture flasks each seeded with 2×10⁶ SF9-cells were infected with 30 μl of the initial virus stock, respectively. After ten days the culture supernatants were harvested as a low scale—high titer viral stock. Then a 500 ml suspension culture of SF9-cells at a density of 2,0×10⁶ cells per milliliter was infected with 5 ml of the second virus stock. Progression of the infection was monitored by determination of the cell viability using the trypan-blue exclusion method. At a cell viability below 10% the viral stock was harvested and virus supernatant separated from cells by centrifugation. The viral titer of this large scale stock had to be determined. For this purpose SF-9 cells were seeded in a 96-well tissue culture plate at a density of 10⁴ cells per well. A total of 24 wells was each infected with one of the following dilutions of the high titer stock: 10 μl of a 1:10⁵ dilution per well, 10 μl of a 1:10⁶ dilution per well and 10 μl of a dilution per well. The volume had to be adjusted to 120 μl per well. After 14 days viability of the cells was determined by the trypan-blue exclusion assay. That dilution with a balanced relation of wells with viable and non-viable cells allows a sufficiently precise estimation of the viral titer which is expected to be 1×10⁸ to 1×10⁹ pfu/ml.

The time course of protein expression was determined at MOIs (multiplicity of infection) of 5 pfu and 10 pfu per cell in an infection experiment with two suspension cultures of SF9 cells at 2,3×10⁶ cells/ml. Samples of the infected cultures were drawn at 24, 48, 72 and 96 hours post infection. These samples were analysed by western blot according to standard protocols. Soluble NKG2D was detected with a peroxidase-conjugated anti-histidine-tag antibody.

Thus, the optimal MOI and the optimal incubation time after infection were used for large scale protein expression in multiple suspension cultures of 500 ml culture volume.

Soluble NKG2D was purified from culture supernatants via its C-terminal histidine tag by affinity chromatography using a Ni-NTA-column as described by Mack (1995) Proc Natl Acad Sci USA 92: 7021.

### EXAMPLE 2

Generation of Monoclonal Antibodies Against Native NKG2D on Human Lymphocytes

Ten weeks old F1 mice from balb/c×C57black crossings were immunized with the soluble extracellular domain of the antigen NKG2D. The antigen was dissolved in 0.9% NaCl at a concentration of 100 μg/ml. The solution was subsequently emulsified 1:2 with complete Freund's adjuvants and 50 μl were injected per mouse intraperitonially. Mice received booster immunizations after 4, 8, and 12 weeks in the same way, except that complete Freund's adjuvants was replaced by incomplete Freund's adjuvants. Ten days after the first booster immunization, blood samples were taken and antibody serum titer against NKG2D antigen was tested by ELISA. Serum titer was more than 1000 times higher in immunized than in not immunized animals. Three days after the second boost, spleen cells were fused with P3×63Ag8.653 cells (ATCC CRL-1580) to generate hybridoma cell lines following standard protocols as described in Current Protocols in Immunology (Coligan, Kruisbeek, Margulies, Shevach and Strober, Wiley Interscience, 1992). After PEG-fusion, cells were seeded at 100.000 cells per well in microtiterplates and grown in 200 μl RPMI 1640 medium supplemented with 10% fetal bovine serum, 300 units/ml recombinant human interleukin 6 and HAT-additive for selection. Culture supernatants from densely grown wells were tested by the following ELISA:

The wells of a 96 U-bottom plate (Nunc, maxisorb) were coated overnight at 4° C. with recombinant NKG2D-antigen at a concentration of 5 μg/ml. Coated-wells were washed three times with washing buffer (0.1M NaCl, 0.05M Na2HPO4 pH 7.3, 0.05% Tween 20, 0.05% NaN3) and subsequently blocked through incubation for one hour at room temperature with 200 pl/well of 2% skimmed milk powder suspended in washing buffer. In the next step, the hybridoma supernatant was incubated undiluted and at several dilutions for two hours at room temperature. After three additional washing steps bound monoclonal antibody was detected with a horseradish peroxidase conjugated polyclonal antibody against mouse immunoglobulin. After 5 times of washing, the ELISA was finally developed by addition of TMB-substrate solution (Tetramethylbenzidine, Roche Mannheim). The colored precipitate was measured after 15 min. at 405 nm using an ELISA-reader.

Supernatants from 10 clones exhibiting strong ELISA-signals were selected for further analysis. In order to identify those hybridoma clones, that produce monoclonal antibodies reactive with native NKG2D-antigen on intact NK-cells and T-lymphocytes, the following flowcytometric analysis was performed:

1×10⁶ PBMC were incubated for 30 min. on ice with 50 μl undiluted hybridoma supernatant and bound monoclonal antibody was detected subsequently detected with fluorescein (FITC) conjugated F(ab′)₂ fragment of a rabbit anti-mouse Ig antibody (Dako Hamburg, Code No. F0313) diluted 1:100 in PBS. In the next step, the free valences of cell-bound FITC-conjugated antibody were blocked through incubation of the cells for 30 minutes with 50 μl mouse serum (Sigma immunochemicals, Deisenhofen, M-5905) diluted 1:10. To distinguish between NK- and T-cells, labeled PBMC were split at this point. One half was stained with a T-cell specific tricolor conjugated anti-CD8 antibody (Caltac Laboratories; Burlingame; USA, Code No. MHCD0306) diluted 1:100; the other half was stained with an NK-cell specific phycoerythrin (PE) conjugated anti-CD56 antibody (Becton Dickinson, Heidelberg, Cat. No. 347747) diluted 1:25. Unlabeled anti-CD16 and anti-CD6 antibodies specifically staining NK-cells or T-lymphocytes, respectively, were used as positive controls of the primary labeling step; a murine monoclonal antibody with irrelevant specificity instead of hybridoma supernatants reactive with recombiant NKG2D served as negative control.

Cells were analyzed by flowcytometry on a FACS-scan (Becton Dickinson, Heidelberg). FACS-staining and measuring of the fluorescence intensity were performed as described in Current Protocols in Immunology (Coligan, Kruisbeek, Margulies, Shevach and Strober, Wiley-Interscience, 1992).

Two-color fluorescence analysis was carried out by applying a positive gate for CD8⁺- and CD56⁺-cells, respectively, thus allowing the detection of FITC-mediated fluorescence separately on CD8⁺-T-lymphocytes and NK-cells. Compared with the distinct staining of CD8⁺-T-lymphocytes and NK-cells with the respective control antibodies, the supernatant of hybridoma cell line 8R23 showed strong reactivity with both NK- and T-cells, whereas two further supernatants were only weakly reactive with both lymphocyte subsets.

Alternatively, monoclonal antibodies against human NKG2D were generated by genetic immunization of mice. For this purpose, two different fragments of human NKG2D from nucleotides (nt) 64 to 462 and from nt 123 to 462 corresponding to amino acid sequences SEQ ID 3 and 4 were PCR-amplified from the cDNA-template shown in FIG. 1, that encode extracellular NKG2D-segments flanked by asparagine (N) and valine (V) or by tryptophan (W) and valine (V), respectively. As PCR-primers the following oligonucleotides were used:

NKG2D-short-f (5′-ATCAAGCTTGTGGATATGTTACAAAAATAACT-3′) (SEQ ID 80) and NKG2D-stop-r (5′-CGCGGTGGCGGCCGCTTACACAGTCCTTTGCATG-3′) (SEQ ID 82) for the amplification of the NKG2D-fragment: nt 123-462

as well as NKG2D-f (5′-ATCAAGCTTGAACCAAGAAGTTCAAATTCC-3′) (SEQ ID 81) and NKG2D-stop-r (5′-CGCGGTGGCGGCCGCTTACACAGTCCTTTGCATG-3′) (SEQ ID 82) for the amplification of the NKG2D-fragment: nt 64-462.

Plasmids for genetic immunization were constructed by cloning each of these PCR-products in-frame into the restriction endonuclease sites Hind III and Not I of the vector VV1 (GENOVAC AG, Germany) as shown in FIG. 4.

The resulting plasmids VV1-NKG2-D (nt 64-462) and VV1-NKG2-D (nt 123-462) allowed the secretion of soluble extracellular NKG2-D fragments tagged by a myc epitope at the N-terminus. The myc epitope was utilized to confirm expression of the soluble NKG2-D fragments. To this end the constructs were expressed by transient transfection into BOSC-23 cells (Onishi (1996) Exp Hematol 24: 324), perforated by addition of Cytoperm/Cytofix (Becton Dickinson); myc-tagged NKG2-D fragments were stained intracellularly by FACScan analysis after reaction with a murine anti-myc monoclonal antibody (9E10, ATCC, CRL-1729) followed by a polyclonal phycoerythrin-labeled rabbit anti-mouse immunoglobulin antibody.

Three 6 to 8 weeks old BALB/c mice were immunized six times with VV1-NKG2-D (nt 64-462) and two mice were immunized three times with VV1-NKG2-D (nt 64-462) followed by three immunizations with VV1-NKG2-D (nt 123-462) using a Helios gene gun (Bio-Rad, Germany) according to a published procedure (Kilpatrick (1998) Hybridoma 17: 569). One week after the last application of the immunization plasmids each mouse was boosted by intradermal injection of 300 μl of recombinant human NKG2-D protein (see Example 1) concentrated 50 μg/ml in phosphate buffered saline without Mg²⁺ and Ca²⁺ ions at the DNA application sites.

Four days later, the mice were killed and their lymphocytes were fused with SP2/0 mouse myeloma cells (American Tissue Type Collection, USA) using polyethylene glycol (HybriMax; Sigma-Aldrich, Germany), seeded at 100,000 cells per well in 96-well microtiter plates and grown in 200 μl DMEM medium supplemented with 10% fetal bovine serum and HAT additive for hybridoma selection (Kilpatrick (1998) Hybridoma 17: 569).

Culture supernatants from densely grown wells were tested by ELISA on immobilized recombinant NKG2D as described above. Supernatants from 122 clones exhibiting positive ELISA-signals were selected for further analysis. In order to identify those hybridoma clones, that produce monoclonal antibodies reactive with native NKGb-antigen on intact NK-cells and CD8⁺ T-lymphocytes, cells were analysed by flowcytometry on a FACS-scan (Becton Dickinson, Heidelberg).

Mononucleated cells from the peripheral blood (PBMC) of a healthy donor were isolated by Ficoll-density gradient centrifugation. In each well of a microtiter plate 200.000 PBMC were incubated with undiluted hybridoma supernatant. After 30 minutes of incubation on ice cells were washed twice with PBS and subsequently stained with fluorescein (FITC)-conjugated F(ab′)₂ fragment of a goat anti-mouse IgG and IgM antibody (Jackson ImmunoResearch Inc. West Grove, USA, Code 115-096-068; 1:100) for 30 minutes on ice. The cells were washed twice with PBS and subsequently stained with two different antibody labeling mixtures. For staining of CD8⁺ T cells, 100.000 PBMC were further incubated for 30 minutes with a phycoerythrin (PE) conjugated CD16 antibody (Becton Dickinson, Heidelberg, Code No. 347617) and a tricolor conjugated CD8 antibody (Caltac Laboratories, Burlingame, USA, Code No. MHCD0806). For staining of NK-cells, the other half of the PBMC was further incubated for 30 minutes with a phycoerythrin (PE) conjugated CD56 antibody (Becton Dickinson, Heidelberg, Code No. 347747) and a tricolor conjugated CD3 antibody (Caltac Laboratories, Burlingame, USA, Code No. MHCD0306.). In order to avoid cross-reactions between the different antibodies within the labeling mixtures mouse serum (Sigma Aldrich, St. Louis, USA, Cat. No. 054H-8958) was added at a final dilution of 1:10.

Triple color fluorescence analysis was carried out by applying a positive gate for CD8⁺ (Tricolor) and a negative gate for CD16⁺ (PE) cells, thus allowing the detection of FITC-mediated fluorescence exclusively attributed to CD8⁺ T-lymphocytes (phenotype: CD8⁺, CD16⁻) without any contaminating signals from CD8⁺ NK-cells. Similarly, triple color fluorescence analysis as carried out by applying a positive gate for CD56⁺, (PE) and a negative gate for CD3⁺-cells (tricolor) thus allowing the detection of FITC-mediated fluorescence exclusively attributed to NK-cells (phenotype: CD56⁺, CD3⁻) without any contaminating signals from CD56⁺-T lymphocytes. As shown in FIG. 5, the supernatants of the hybridomas designated 11B2, 8G7 and 6E5 contained monoclonal antibodies reactive with native NKG2D on the surface of both human CD8⁺ T-lymphocytes and NK-cells. Staining with supernatant of the hybridoma 10H9 is shown as a representative example of many monoclonal antibodies reactive with immobilized recombinant NKG2D, that were, however, not capable of binding the native NKG2D-receptor complex on intact cells. FACS staining and measuring of the fluorescence intensity were performed as described in Current Protocols in Immunology (Coligan, Kruisbeek, Margulies, Shevach and Strober, Wiley-Interscience, 1992).

The hybridomas producing antibodies reacting with NKG2-D on CD56⁺ NK- and CD8⁺ T cells were subcloned once by limited dilution on 96-well microtiter plates. Positive subclones were identified by flowcytometry on NKG2D-positive NKL-cells (Bauer (1999) Science 285: 727) incubated with supernatants harvested from wells showing cell growth. Cell-bound monoclonal antibody was detected with the fluorescein (FITC) conjugated F(ab′)₂-fragment of a rabbit anti-mouse Ig antibody (Dako, Hamburg, Code No. F0313). The subclones 11B2D10, 8G7C10 and 6E5A7 were further used for the construction of NKG2D-directed bispecific antibodies (see Example 3).

### EXAMPLE 3

Construction of Bispecific Single-Chain Antibodies Anti-NKG2D×Anti-EpCAM

The bispecific antibodies were constructed as depicted in FIG. 2. The variable regions V_{L} and V_{H} of those antibodies binding to native NKG2D on intact cells were cloned from total RNA of the corresponding hybridoma cell lines as described by Orlandi (1989) Proc. Natl. Acad. Sci. USA 86: 3833, except that the PCR-fragments of variable regions amplified from hybriomas 11B2D16 (SEQ ID 7-16), 8G7C10 (SEQ ID 27-36), 6E5A7 (SEQ ID 37-46) and 6H7E7 (SEQ ID 17-26) were directly the TA-cloning vector GEM-T Easy (Promega, Cat. No. A1360). Subsequently, cloned VL- and VH-regions served as templates for a two-step fusions-PCR resulting in the corresponding scFv-fragments with the domain arrangement VL/VH. The VL-specific primer pair used for this purpose consists of oligonucleotides 5′V_{L}B5RRV (5′AGG TGT ACA CTC CGA TAT CCA GCT GAC CCA GTC TCC A 3′ (SEQ ID 83)) and 3═VLGS15 (5′GGA GCC GCC GCC GCC AGA ACC ACC ACC ACC TTT GAT CTC GAG CTT GGT CCC3′ (SEQ ID 84)), the VH-primer pair of oligonucleotides 5′V_{H}GS15 (5′GGC GGC GGC GGC TCC GGT GGT GGT GGT TCT CAG GT(GC) (AC)A(AG) CTG CAG (GC)AG TC(AT) GG 3′ (SEQ ID 85)) and 3′VHBspEI (5′AAT CCG GAG GAG ACG GTG ACC GTG GTC CCT TGG CCC CAG 3′ (SEQ ID 86)). In the first PCR step VH- and VL-amplification products were obtained with the following PCR-programm: denaturation at 94° C. for 5 min, annealing at 37° C. for 2 min, elongation at 72° C. for 1 min for the first cycle; denaturation at 94° C. for 1 min, annealing at 37° C. for 2 min, elongation at 72° C. for 1 min for 6 cycles; denaturation at 94° C. for 1 min, annealing at 55° C. for 1 min, elongation at 72° C. for 45 sec and 18 cycles; termial extension at 72° C. for 2 min. For the second step of the fusion PCR VH- and VL-PCR fragments were purified from agarose gel, mixed with oligonucleotide primers 5′VLB5RRV and 3′VHBspEI, and subjected to the following PCR-programm: denaturation at 94° C. for 5 min once; denaturation at 94° C. for 1 min, annealing at 55° C. for 1 min, elongation at 72° C. for 1,5 min and 8 cycles; terminal extension at 72° C. for 2 min. VL/VH-fusion products encoding anti-NKG2D scFv-fragments were purified from agarose gele, and digested with the restriction enzymes BsrGI/BspEI. The mammalian expression vector pEF-DHFR (Mack (1995) Proc Natl Acad Sci USA 92: 7021) containing an EcoRI/SalI-cloned DNA-fragment described in WO0003016, FIG. 10 was also digested with the restriction enzymes BsrGI/BspEI releasing a 750 bp-fragment; the remaining vector-fragment was gele purified and used for cloning of the anti-NKG2D scFv-fragments.

Thus, the resulting derivatives of mammalian expression vector pEF-DHFR contain EcoRI/SalI-DNA inserts encoding bispecific single-chain antibodies as described by Mack (1995) Proc Natl Acad Sci USA 92: 7021, that are directed against NKG2D and EpCAM. EpCAM is expressed by many epithelial tumors and already used as target antigen for the adjuvant treatment of resected colorectal cancer with a murine monoclonal antibody.

The expression plasmids for anti-NKG2D×anti-EpCAM bispecific single-chain antibodies (SEQ ID 47-49) were transfected into DHFR-deficient CHO-cells by electroporation; selection for stable transfectants, gene amplification and protein production were performed as described (Mack (1995) Proc Natl Acad Sci USA 92: 7021). Bispecific antibody was purified from culture supernatant via the C-terminal histidine tag by using Ni-NTA-column as described (Mack (1995) Proc Natl Acad Sci. USA 92: 7021, see also FIG. 3).

### EXAMPLE 4

Antibodies Directed to the Extracellular Domain of DAP-10

Antibodies reactive with the extracellular domain of the NKG2D-receptor complex can be also be obtained with the following protocol:

6 to 8 weeks old BALBlc mice may be immunized with a peptide corresponding to the complete extracellular domain of human DAP-10 comprising 30 amino acids (SEQ ID 5, QTTPGERSSLPAFYPGTSGSCSGCGSLSLP) or a part thereof (Wu (1999) Science 285: 730), conjugated with a carrier protein, respectively. For example, a peptide comprising the 21 N-terminal amino acids of the extracellular domain of DAP10 (SEQ ID 6, QTTPGERSSLPAFYPGTSGSC) may be coupled to maleinimide activated KLH in a directed manner via the mercapto-group of its C-terminal cystein. The conjugate may be dissolved in 0,9% NaCl at a concentration of 100 μg/ml, the solution subsequently emulsified 1:2 with complete Freund's adjuvants and 50 μl per mouse infected intraperitonially. Mice may receive booster immunizations resembling the primary immunization after 4, 8 and 12 weeks, except that complete Freund's adjuvants can be replaced by incomplete Freund's adjuvants. Ten days after the first booster immunization, blood samples may be taken and antibody serum titer tested by ELISA on immobilized BSA conjugated with the 21-mer DAP-10 peptide as described above for KLH.

Three days after the second boost, spleen cells from mice with positive serum titer may be fused with P3×63Ag8.653 cells (ATCC CRL-1580) to generate hybridoma cell lines following standard protocols as described in Current Protocols in Immunology (Coligan, Kruisbeek, Margulies, Shevach and Strober, Wiley-Interscience, 1992). After PEG-fusion, cells may be seeded at 100.000 cells per well in microtiterplates and grown in 200 μl RPMI 1640 medium supplemented with 10% fetal bovine serum, 300 units/ml recombinant human interleukin 6 and HAT-additive for selection. Culture supernatants from densely grown wells may be tested for reactivity with DAP10-peptide by the following ELISA:

The wells of a 96 U-bottom plate (Nunc, maxisorb) are coated overnight at 4° C. with peptide-BSA-conjugate at a concentration of 5 μg/ml. Coated wells are washed three times with washing buffer (0.1M NaCl, 0.05M Na₂HPO₄ pH 7.3, 0.05% Tween 20, 0.05% NaN₃) and subsequently blocked through incubation for one hour at room temperature with 200 μl/well of 2% skimmed milk powder suspended in washing buffer. In the next step, hybridoma supernatant is incubated undiluted and at several dilutions for two hours at room temperature. After three additional washing steps bound monoclonal antibody can be detected with a horseradish peroxidase conjugated polyclonal antibody against mouse immunoglobulin. After 5 times of washing, the ELISA can be finally developed by addition of TMB-substrate solution (Tetramethylbenzidine, Roche). The colored precipitate is measured after 15 min. at 405 nm using an ELISA-reader.

In order to identify those peptide-reactive hybridoma clones, that produce monoclonal antibodies capable of binding to DAP-10 within the NKG2D-receptor complex on intact NK-cells and CD8⁺ T-lymphocytes, the triple color fluorescence analysis on PBMC may be performed that is described in Example 2.

The variable regions of monoclonal antibodies staining intact NK-cells and CD8⁺ T-lymphocytes may be cloned from the corresponding hybridoma cell lines and used for construction of bispecific single-chain antibodies as described in Example 3.

### EXAMPLE 5

Enhanced Priming of Naive CD8⁺ T Cells by NKG2D-Directed Antibodies

For in vitro priming experiments naive human CD8⁺ T cells were isolated as follows:

Mononuclear cells (PBMC) were prepared by Ficoll density centrifugation from 500 ml peripheral blood obtained from a healthy donor. CD8⁺-T cells were isolated by negative selection using a commercially available cell separation kit (R&D Systems, HCD8C-1000). The CD8⁺-T cell column was loaded with 2×10⁸ PBMC, which had been preincubated with the manufacture's antibody cocktail supplemented with 1 μg of a monoclonal anti-CD11b antibody (Coulter 0190) per column. Since primed non-proliferating cytotoxic CD8⁺ T cells share the CD45RA⁺/RO⁻ phenotype with naive CD8⁺ T lymphocytes, CD11b was introduced as additional cell purification marker in order to get rid of the former T cell subset. Thus, only CD11b⁻/CD8⁺ T cells entered the purification procedure based on CD45-isoforms finally resulting in naive CD8⁺-T lymphocytes, that like naive CD4⁺-T cells carry the CD45RA⁺/RO⁻ phenotype. Successful purification of CD8⁺-T cells was controlled by flowcytometry after single staining with an anti-CD8 antibody. Absence of CD11b⁺-cells from CD8⁺-T cell preparations was confirmed by single staining with an anti-CD28 antibody, since CD11b-positive CD8⁺-T cells are always CD28-negative and vice versa.

CD45RO⁺-cells were removed from purified CD8⁺-T cells through incubation with a murine monoclonal anti-CD45RO antibody (PharMingen, UCHL-1, 31301) followed by magnetic beads conjugated with a polyclonal sheep anti-mouse Ig antibody (Dynal, 110.01). The purity of the remaining naive CD8⁺-T cells proved to be >95% as determined by flowcytometry after double staining with anti-CD45RA/anti-CD45RO. The average yield of naive CD8⁺ T cells per 500 ml peripheral blood was 5×10⁶ (CD8).

The in vitro priming experiment with naive CD8⁺ T cells was carried out as follows:

25.000 EpCAM-transfected CHO-cells per well were incubated in a 96-well flat-bottom culture plate for 2 hours, that had been coated overnight with a polyclonal rabbit anti-mouse IgGl antibody (Dako, Z0013) diluted 1:1000 in PBS. After the cells had adhered to the plastic, they were irradiated with 14.000 rad. Subsequently, 50.000 purified naive CD8⁺ T cells per well were added in RPMI 1640 medium supplemented with 10% human AB serum, 100 U/ml penicillin, 100 mg/ml streptomycin, 2 mM glutamin, 1 mM sodium pyruvat, 10 mM HEPES-buffer, 1× non-essential amino acids (Gibco) and 50 μM β-mercaptoethanol. The EpCAM-specific B7-1/4-7 single-chain construct described in WO9925818 (Example 7) was added at 500 ng/mI together with 1 μg/ml of a murine IgG1 isotype control (Sigma, M-7894) and either 250 ng/ml, 50 ng/ml or no bispecific single-chain antibody (bsc) EpCAM×CD3 (Mack (1995) Proc. Natl. Acad. Sci. U.S.A. 92: 7021). 500 ng/mI of the B7-1/4-7 single-chain construct was the maximum concentration that did not yet by itself affect CD45-isoform expression on CD8⁺-T cells. In a parallel experiment, the same concentrations and combinations of bsc EPCAM×CD3 and B7-1/4-7 single-chain construct were used except that the IgG1 isotype control was replaced by diluted hybridoma supernatant kindly provided by Dr. Moretta, Genova, Italy containing the murine NKG2D-specific IgG1 antibody BAT221 at a final concentration of 1 μg/ml. Alternatively the NKG2D-specific monoclonal antibody can be exchanged for a bispecific antibody binding to NKG2D and EpCAM like SEQ ID 47-49 and 72-79. In contrast to the monoclonal antibody no bispecific antibody is immobilized on the solid support.

All experiments were carried out with triplicates of identical wells. Furthermore, a set of two identical 96-well plates was prepared in order to make sure, that enough cells were available for flowcytometry on days 3 and 6. On day 3, supernatant was harvested from one 96-well plate and TNF-α concentration determined by using a commercially available ELISA-kit (PharMingen, 2600KK). The cells were also harvested and subjected to flowcytometric analysis of CD45-isoform expression. Moreover, half of the supernatant was removed from each well of the second 96-well plate and replaced by fresh medium adjusted to the corresponding concentrations of B7-1/4-7 single-chain construct, bsc EpCAM×CD3, BAT221 and/or isotype control. On day 6, the cells of this 96-well plate were harvested and their CD45-isoform expression pattern was analyzed by flowcytometry. In general, cells and supernatants from three identical wells (triplicates) were pooled for flowcytometry and cytokine analysis, respectively.

Flowcytometry was performed on a FACScan (Becton Dickinson). Flowcytometric analysis of CD45-isoform expression was carried out by double staining of 1×10⁵ cells with a PE-conjugated monoclonal anti-CD45RA antibody (Coulter, 2H4, 6603904) and a FITC-conjugated monoclonal anti-CD45RO antibody (DAKO, UCHL-1, F 0800) for 30 minutes on ice. Flowcytometric monitoring of T cell purification was equally carried out by single stainings with a Tricolor-conjugated monoclonal anti-CD8 antibody (Medac, MHC0806) and a FITC-conjugated monoclonal anti-CD28 antibody (Medac, MHCD2801).

In these priming experiments, the primary signal was mediated by the bispecific single-chain antibody (bscAb) EpCAM×CD3 thus imitating specific antigen recognition through the T-cell receptor (TCR); the second or costimulatory signal was mediated by the EpCAM-specific B7-1/4-7 single-chain construct through engagement of CD28 on the T-cells. Thus the effect of an NKG2D-directed antibody on the priming of naive CD8⁺ T cells could be determined, that were activated through the TCR-like and the costimulatory signal. Non-human stimulator cells armed with EpCAM-specific constructs were used in order to avoid background signals, that may arise with human stimulator cells incidently expressing costimulatory receptors. The kinetics of T cell priming was monitored by flowcytometry on days 3 and 6 simultaneously measuring the expression of CD45RA and CD45RO. As shown in FIG. 6, almost the entire population of naive T cells changed the CD45-phenotype to that of primed T cells, i.e. CD45RA⁻/RO⁺, within 6 days in the presence of B7-1/4-7 single-chain construct (500 ng/ml) and bscAb EpCAM×CD3 (250 ng/ml). Accordingly, an intermediate state could be observed on day 3. Surprisingly, the additional presence of the NKG2D-directed antibody further accelerated proliferation and priming of naive CD8⁺ T cells. This could be concluded from the higher percentage of CD8⁺ T-cells located on day 3 in the lower right quadrant in FIG. 6B, if the cells had received the additional NKG2D-signal compared to a smaller percentage when the naive T-cells were stimulated through the TCR-like and costimulatory signal alone. Since TNF-α is typically produced by primed CD8⁺-T cells but not by their naive counterparts, the effect of enhanced T-cell priming could be confirmed by higher concentrations of TNF-α measured on day 3 in the supernatant of CD8⁺ T-cells receiving the NKG2D-signal compared to those primed in the absence of the NKG2D-directed antibody (FIG. 7).

Even the flowcytometric results on day 6 (FIGS. 6C and E), when the priming process was almost completed, showed the NKG2D-mediated support of T cell priming: The loss of CD45RA-expression within 6 days proved to be more profound in the presence than in the absence of an NKG2D-mediated signal as measured by the higher percentage of CD8⁺ T cells located within the lower right field in FIG. 6C.

### EXAMPLE 6

NKG2D-Directed Antibodies Enhance the Cytotoxicity of CD8⁺ T-Cells and NK-Cells Triggered through Engagement of the TCR- or the FcγRIII-Complex, Respectively

In order to test the recruitment of cytotoxic lymphocytes i.e. CD8⁺ T cells and NK cells by NKG2D-directed antibodies, we performed ⁵¹Cr-release assays using the murine FcγR-positive P815 cell line as target and either a Melan-A specific human CD8⁺ T cell clone (Melan-A cells) or NKL cells (Bauer (1999) Science 285: 727) as effectors. The ⁵¹Cr-release assay measuring cellular cytotoxicity was carried out as described by Mack (1995) Proc Natl Acad Sci USA 92: 7021 with minor modifications. 10.000 ⁵¹Cr-labeled P815-cells were either mixed with 50.000 Melan-A cells or with 200.000 NKL cells per well of a round-bottomed microtiter plate. NKL cells were incubated for 4 h in the presence of 5μg/ml CD16 antibody (3G8) and/or diluted hybridoma supernatant containing the murine NKG2D-specific monoclonal antibody BAT221. Melan A cells were incubated for 4 h in the presence of 0,2 μg/ml CD3 antibody (OKT3) and/or diluted BAT221-supernatant. Maximum ⁵¹Cr-release was determined by lysis of target cells with Maly buffer (2% SDS/0,37% EDTA/0,53% Na₂CO₃). The spontaneous ⁵¹Cr-release was determined with target cells incubated in the absence of effector cells and antibodies. Target cells incubated with effector cells in the absence of antibodies served as negative control. Specific lysis was calculated as [(cpm, experimental release)−(cpm, spontaneous release)]/[(cpm, maximal release)−(cpm, spontaneous release)]. The cytotoxicity assay was carried out with triplicate samples. As shown in FIG. 8, BAT221 did not induce any substantial target cell lysis by itself in contrast to published NKG2D-antibodies (Bauer (1999) Science 285: 727). As expected the CD16- and the CD3-antibody induced redirected target cell lysis with NKL-cells and Melan A-cells, respectively. However, although not cytotoxic by itself, BAT221 surprisingly enhanced target cell cytotoxicity triggered by engagement of the TCR-complex on Melan A-cells and of the FcγRIII-complex on NKL-cells. Alternatively, P815-cells can be replaced e.g. by EpCAM-positive Kato-cells and the NKG2D-specific monoclonal antibody exchanged for a bispecific antibody binding to NKG2D and EPCAM like SEQ ID 47-49 and 72-79. The TCR-complex on CD8⁺ T-cells may be engaged by a bispecific antibody binding to CD3 and to a surface antigen on the target cells or by specific TCR-recognition of processed MHC I-complexed target cell antigen. The FcγRIII-complex on NK-cells may be engaged by a bispecific antibody binding to CD16 and to a surface antigen on the target cells or by a target cell specific monoclonal antibody like e.g. a human EpCAM antibody bound to FcγRIII via its Fc part.

### EXAMPLE 7

Bispecific Single-Chain Antibodies with an NKG2D-Binding Site Located C-Terminally of the Target Binding Site

Five Balb/c mice were genetically immunized with human NKG2D as described in Example 2. In order to identify mice with a serum antibody reactivity on the surface of human lymphocytes resembling the expression pattern of the NKG2D-receptor complex, a triple fluorescence analysis on PBMC as described in Example 2 was carried out with mouse serum diluted 1:10, 1:20 and 1:40. As shown in FIG. 9, only one mouse serum (No. 4) exhibited strong staining of both CD8⁺ T-lymphocytes and NK-cells. The spleen cells of this mouse were used as an immunoglobulin repertoire for the construction of a combinatorial antibody library as described in WO9925818 (Example 6). The cloned antibody repertoire was displayed on filamentous phage as an N2-VH-VL-fusion protein, imitating the C-termial position of the corresponding antigen binding site within a bispecific single-chain antibody. Selection of NKG2D-reactive scFv-fragments was carried out through two rounds of library panning on immobilized recombinant NKG2D-protein as described in WO9925818 for the 17-1A- or EpCAM-antigen followed by three rounds of panning on NKG2D-positive NKL-cells. Cell panning was carried out in PBS/10%FCS by resuspending 2-5×10⁶ NKL-cells in 500 μl phage suspension followed by 45 minutes of moderate shaking at 4° C. Then cells plus bound phage particles were centrifuged in a desk centrifuge at 2500 rpm for 2 minutes. Then the resulting pellet was washed twice by resuspension in 1 ml of PBS/10%FCS followed by recentrifugation 4-third round of panning). During the fourth round of panning 3 washing steps were applied as well as 5 washing steps during the fifth round of panning. Specifically bound phage particles were eluted from the NKL-cells by resuspension and incubation in 500 μl HCl-Glycin for 10 minutes followed by neutralization with 30 μl 2M Tris-base (pH 12). The eluant were used for infection of a new uninfected E. coli XL1 Blue culture. After five rounds of phage-production and subsequent selection for antigen-binding scFv-displaying phages, plasmid DNAs from E. coli cultures were isolated corresponding to the fourth and fifth round of panning. For the production of soluble scFv-antibody fragments that carry the N2-domain at their N-terminus, the DNA fragment encoding the CT-domain of the geneIII-product was excised with SpeI/NotI and replaced by the tetramerization domain of human p53 (Rheinnecker (1996) J Immunol. 157: 2989) flanked by an N-terminal Ig-hinge-region and a C-terminal Flag-epitope (FIG. 10, SEQ ID 50 and 51). After ligation, the resulting pool of plasmid DNA was transformed into 100 μl of heat shock competent E. coli XL1 Blue cells and plated on Carbenicilline LB-agar. Single colonies were check by screening-PCR for integrity of the cloned VH- and VL-regions and those with intact variable regions subjected to periplasmatic expression of soluble antibody fragments as described in WO9925818 (Example 6). The periplasma preparations were tested by ELISA on immobilized recombinant NKG2D and specifically binding N2-scFv-p53-fusion proteins detected with the peroxidase-conjugated anti-Flag M2 antibody (Sigma, A-8592). As shown in FIG. 11 many NKG2D-reactive clones from the fourth and fifth round of panning could be identified. The scFv-encoding fragments of the positive clones were excised with BspEI from the phage display vector and subcloned into the plasmid vector BS-CTI (see WO 00-06605, FIG. 2) prepared by digestion with BspEI and XmaI followed by dephosphorylation with calf intestinal phophatase. The correct orientation of the scFv-fragments was checked by analytic digestion with the restriction enzymes BspEI and SpeI. By insertion into BS-CTI the scFv-encoding fragments were fused in-frame to a His₆-tag (SEQ ID 52-71). In the next step, the scFv-fragments were excised with BspEI and SalI from BS-CTI and subcloned BspEI/SalI into the mammalian expression vector PEF-DHFR that contained an EpCAM-specific, CD3-directed bispecific single-chain antibody as described by Mack (1995) Proc Natl Acad Sci USA 92: 7021 except that the scFv-fragment of the EpCAM-specific monoclonal antibody M79 had been replaced by that of the monoclonal antibody 3B10, that binds to EpCAM with high affinity (=bsc 3B10×CD3). Thus, the CD3-specific scFv-fragment was replaced by NKG2D-reactive scFv-fragments resulting in EpCAM-specific, NKG2D-directed bispecific single-chain antibodies (SEQ ID 72-79).

CHO/dhfr− cells were chosen for the transient expression of the antibody-like molecules. The transfection of the cells was performed with the TransFast transfection reagent (Promega, Heidelberg) according to the manufacture's protocol. Briefly, 2.5×10⁵ cells were seeded per well in six-well plates 20 hrs prior to transfection. The transfection mix was prepared by adding 6 μg of plasmid DNA harboring the antibody sequences or the β-galactosidase gene to 1 ml MEM alpha media without supplements. After mixing 30 μl of TransFast reagent were added. The mix was vortexed and incubated for 15 minutes at room temperature. Then, the media was removed from the cells and replaced by the transfection mix. After 1 hour incubation period at 37° C. the transfection mix was aspirated and fresh complete MEM alpha was added to the cells. Protein production was analyzed 4 to 5 days post transfection by FACS analysis. The supernatants were harvested after 4 to 5 days. To remove cell debris the supernatants were centrifuged. The function of the antibodies was analyzed in binding studies of the anti-EpCAM specific part on Kato III cells. Per sample, 4×10⁵ cells were incubated in 75 μl transfected cell supernatant diluted with 25 μl FACS buffer (1% heat-inactivated FBS, 0.05% Na3N in PBS). The samples were incubated for 30 minutes at 4° C. After washing the cells twice with 200 μl FACS buffer the cells were incubated with 2 μg/ml anti-Penta.His antibody (QIAGEN, Netherlands) for 30 minutes at 4° C. Subsequently, the cells were washed again and incubated for 30 minutes with a sheep anti-mouse FITC conjugate (SIGMA, Deisenhofen. Binding was detected with a FACS Calibur (Becton-Dickinson) (FIG. 12). Supernatants of CHO-cells transiently transfected with bsc 3B10×P4-3 and bsc 3B10×P5-2 showed positive ELISA-signals on immobilized recombinant NKG2D-antigen (FIG. 13).

As an alternative to NKG2D, DAP10-peptide-conjugates as described in Example 4 may be used for immunizing mice, whose spleen cells may be the source of an immunoglobulin repertoire for the construction of a combinatorial antibody library like that described in this Example. Thus, DAP10 reactive antibody binding sites recognizing the NKG2D-receptor complex on CD8⁺ T-lymphocytes and NK-cells even when located C-terminally of the target binding site within a bispecific single-chain antibody may be selected through library panning on immobilized peptide-conjugate and/or cells expressing the NKG2D-receptor complex.

### EXAMPLE 8

Recruitment of CD8⁺ T and NK Effector Cells by Bispecific Single-Chain Antibodies with an NKG2D-Binding Site Located C-Terminally of the Target Binding Site

In order to test the recruitment of cytotoxic lymphocytes i.e. CD8⁺ T cells and NK cells by NKG2D-directed bispecific antibodies, we performed ⁵¹Cr-release assays using the gastric cancer cell line Kato as target and either a Melan-A specific human CD8⁺ T cell clone (Melan-A cells), NKL cells (Bauer (1999) Science 285: 727) or unstimulated PBMC from a healthy donor as effectors.

The ⁵¹Cr-release assay measuring cellular cytotoxicity redirected against EpCAM-positiv Kato-cells was carried out as described by, Mack (1995) Proc Natl Acad Sci USA 92: 7021 with minor modifications. 10.000 ⁵¹Cr-labeled Kato cells were either mixed with 50.000 Melan-A cells or with 200.000 NKL cells or PBMC per well of a round-bottomed microtiter plate and incubated for 4 h (Melan A- and NKL-cells) or 18 h (PBMC) in the presence of culture supernatant from CHO cells diluted 1:2 that had been transfected with different EpCAM-specific, NKG2D-directed bispecific single-chain antibodies (3B10×P4-3, 3B10×P4-14, 3B10×P5-2 and 3B10×P5-23) described in Example 8. Maximum ⁵¹Cr-release was determined by lysis of target cells with Maly buffer (2% SDS/0,37% EDTA/0,53% Na₂CO₃). The spontaneous ⁵¹Cr-release was determined with target cells incubated in the absence of effector cells and bispecific antibody. Target cells incubated with effector cells in the absence of antibodies served as negative control. Specific lysis was calculated as [(cpm, experimental release)−(cpm, spontaneous release)]/[(cpm, maximal release)−(cpm, spontaneous release)]. The cytotoxicity assay was carried out with triplicate samples. As shown in FIG. 14, the supernatant of CHO-cells transiently transfected with the NKG2D-directed bispecific single-chain antibody 3B10×P4-3 induced weak but reproducible and titratible cytolyis of EpCAM-positive Kato-cells with both Melan-A- and NKL-cells in the 4h-⁵¹Cr release test. Moreover, the supernatants of CHO-cells transiently transfected with the NKG2D-directed bispecific single-chain antibodies 3B10×P4-3, 3B10×P4-14, 3B10×P5-2 and 3B10×P5-23, respectively induced substantial target cell lysis with PBMC in the 18 h ⁵¹Cr-release assay (FIG. 15).

1. A multifunctional polypeptide comprising
(a) a first domain comprising a binding site specifically recognizing an extracellular epitope of the NKG2D receptor complex; and
(b) a second domain having receptor or ligand function.

2. The multifunctional polypeptide of claim 1 wherein said binding site is the binding site of an immunoglobulin chain.

3. The multifunctional polypeptide of claim 1 wherein said binding site is a natural NKG2D-ligand of said receptor complex.

4. The multifunctional polypeptide of claim 3 wherein said natural NKG2ligand is selected from the group consisting of MIC-A, MIC-B, ULBP-1 and ULBP-2.

5. The multifunctional polypeptide of claims 1 to 4 wherein said binding site specifically recognizes an extracellular epitope of NKG2D or of DAP10.

6. The multifunctional polypeptide of any one of claims 1 to 5 wherein said receptor or ligand function is an antigen binding site of antibodies or fragments or derivatives thereof against (i) tumor associated antigens, (ii) antigens of infective agents or (iii) surface markers of sub-populations of cells such as differentiation antigens (CD antigens), natural ligands or receptors or fragments thereof or modifications thereof that interact with said tumor associated antigens or surface markers, preferably (i) heregulins, binding to the tumor associated antigens erbB-2,-3 and -4, (ii) CD4 that interacts with gp 120 of HIV infected cells or (iii) melanocyte stimulating hormone (MSH) that binds to the MSH receptor on melanocytes and tumors derived therefrom (maligned melanomes) or chemokines binding to corresponding chemokine receptors, or MHC molecules or fragments thereof complexed with peptides that bind to T-cell receptors of predefined specificity and thus recognize certain T-cell sub-populations or antigen binding sites of T-cell receptors that specifically interact with predefined MHC peptide complexes or NKp46 which interacts with haemagglutinin (HA) of influenza virus.

7. The multifunctional polypeptide of claim 6 wherein said tumor-associated antigen is selected from the group consisting of Lewis Y, Muc-1, erbB-2,-3 and -4, Ep-CAM, EGF-receptor (e.g. EGFR type I or EGFR type II), EGFR deletion neoepitope, CA19-9, Muc-1, LeY, TF-, Tn- and sTn-antigen, TAG-72, PSMA, STEAP, Cora antigen, CD7, CD19 and CD20, CD22, CD25, Ig-α and Ig-β, A33 and G250, CD30, MCSP and gp100, CD44-v6, MT-MMPs, (MIS) receptor type II, carboanhydrase 9, F19-antigen, Ly6, desmoglein 4, PSCA, Wue-1, GD2 and GD3 as well as TM4SF-antigens (CD63, L6, CO-29, SAS) or the alpha and gamma subunit of the fetal type acetylcholinreceptor (AChR).

8. The multifunctional polypeptide of claim 6 wherein said surface marker for an infected cell is selected from the group consisting of viral envelope antigens, e.g. of human retroviruses (HTLV I and II, HIV1 and 2) or human herpes viruses (HSV1 and 2, CMV, EBV), haemagglutinin e.g. of influenza virus A, B or C, glycoprotein E1 and E2 of rubella virus or RGP of rabies virus.

9. The multifunctional polypeptide of any one of claims 1 to 8 which is a bi-specific antibody.

10. The multifunctional polypeptide of claim 9 wherein said multifunctional polypeptide is selected from the group consisting of a synthetic, a chimeric and a humanized antibody.

11. The multifunctional polypeptide of any one of claims 1 to 10 which is a single-chain.

12. The multifunctional polypeptide of any one of claims 1 to 10 wherein said two domains are connected by a polypeptide linker.

13. The multifunctional polypeptide of any one of claims 1 to 12 wherein said first and/or second domain mimic or correspond to a V_{H} and V_{L} region of a natural antibody.

14. The multifunctional polypeptide of any one of claims 1 to 13 wherein at least one of said domains is a single-chain fragment of the variable region of the antibody.

15. The multifunctional polypeptide of any one of claims 1 to 14 wherein said domains are ranged in the order V_{L}NKG2D-V_{H}NKG2D-V_{H}TA-V_{L}-TA or V_{L}TA-V_{H}TA-V_{H}NKG2D-V_{L}NKG2D, wherein the TA represents a target antigen.

16. The multifunctional polypeptide of claim 15 wherein said target antigen is selected from the group consisting of Lewis Y, Muc-1, erbB-2,-3 and -4, Ep-CAM, EGF-receptor (e.g. EGFR type I or EGFR type II), EGFR deletion neoepitope, CA19-9, Muc-1, LeY, TF-, Tn- - and sTn-antigen, TAG-72, PSMA, STEAP, Cora antigen, CD7, CD19 and CD20, CD22, CD25, Ig-α and Ig-β, A33 and G250, CD30, MCSP and gp100, CD44-v6, MT-MMPs, (MIS) receptor type II, carboanhydrase 9, F19-antigen, Ly6, desmoglein 4, PSCA, Wue-1, GD2 and GD3 as well as TM4SF-antigens (CD63, L6, CO-29, SAS) or the alpha and gamma subunit of the fetal type acetylcholinreceptor (AChR).

17. The multifunctional polypeptide of any one of claims 12 to 16 wherein said polypeptide linker comprises a plurality of glycine, serine and/or alanine residues.

18. The multifunctional polypeptide of any one of claims 12 to 17 wherein said polypeptide linker comprises a plurality of consecutive copies of an amino acid sequence.

19. The polypeptide of any one of claims 12 to 18 wherein said polypeptide linker comprises 1 to 5, 5 to 10 or 10 to 15 amino acid residues.

20. The multifunctional polypeptide of any one of claims 4 to 19 wherein said polypeptide linker comprises the amino acid sequence Gly-Gly-Gly-Gly-Ser.

21. The multifunctional polypeptide of any one of claims 1 to 20 comprising at least one further domain.

22. The multifunctional polypeptide of claim 21 wherein said further domain is linked by covalent or non-covalent bonds.

23. The multifunctional polypeptide of claims 21 or 22, wherein said at least one further domain comprises an effector molecule having a conformation suitable for biological activity, capable of sequestering an ion or selective binding to a solid support or to a preselected determinant.

24. The multifunctional polypeptide of any one of claims 21 to 23 wherein said further domain confers a co-stimulatory and/or a co-activating function.

25. The multifunctional polypeptide of claim 24 wherein said co-stimulatory function is mediated by a CD28-ligand or a CD137-ligand.

26. The multifunctional polypeptide of claim 25 wherein said CD28-ligand or CD137-ligand is B7-1 (CD80), B7-2 (CD86), an aptamer or an antibody or a functional fragment or a functional derivative thereof.

27. A polynucleotide which upon expression encodes a multifunctional polypeptide and/or functional parts of a multifunctional polypeptide of any one of claims 1 to 26.

28. A vector comprising the polynucleotide of claim 27.

29. A cell transfected with the polynucleotide of claim 27 or the vector of claim 28.

30. A method for the preparation of the multifunctional polypeptide and/or parts of the multifunctional polypeptide of any one of claims 1 to 26 comprising culturing a cell of claim 29 and isolating said multifunctional polypeptide or functional parts thereof from the culture.

31. A composition comprising the polypeptide of any one of claims 1 to 26, the polynucleotide of claim 27 or the vector of claim 28.

32. The composition of claim 31 further comprising a molecule conferring a co-stimulatory and/or co-activating function.

33. The composition of claim 31 wherein said co-stimulatory function is mediated by a CD28-ligand or a CD137-ligand.

34. The composition of claim 31 wherein said CD28-ligand or CD137-ligand is B7-1 (CD80), B7-2 (CD86), an aptamer or an antibody or a functional fragment or a functional derivative thereof.

35. The composition of any one of claims 31 to 34 which is a pharmaceutical composition optionally further comprising a pharmaceutically acceptable carrier.

36. The composition of any one of claims 31 to 35 which is a diagnostic composition optionally further comprising suitable means for detections.

37. Use of the multifunctional polypeptide of any one of claims 1 to 26, the polynucleotide of claim 27 or the vector of claim 28 for the preparation of a pharmaceutical composition for the treatment of cancer, infections and/or autoimmune conditions, cancer, i.e. maligne (solid) tumors and hematopoietic cancer forms (leukemias and lymphomas), benigne tumors such as benigne hyperplasia of the prostate gland (BPH), autonomous adenomes of the thyroid gland or of other endocrine glands or adenomas of the colon; initial stages of the malignancies, infectious diseases, caused by viruses, bacteria, fungi, protozoa or helmints, auto immune diseases wherein the elimination of the subpopulation of immune cells is desired that causes the disease; prevention of transplant rejection or allergies.

38. The use of claim 37 wherein said infection is a viral, a bacterial or a fungal infection, wherein said cancer is a head and neck cancer, gastric cancer, oesaphagus cancer, stomach cancer, colorectal cancer, coloncarcinomaa, cancer of liver and intrahepatic bile ducts, pancreatic cancer, lung cancer, small cell lung cancer, cancer of the larynx, breast cancer, mamma carcinoma, malignant melanoma, multiple myeloma, sarcomas, rhabdomyosarcoma, lymphomas, folicular non-Hodgkin-lymphoma, leukemias, T- and B-cell-leukemias, Hodgkin-lymphoma, B-cell lymphoma, ovarian cancer, cancer of the uterus, cervical cancer, prostate cancer, genital cancer, renal cancer, cancer of the testis, thyroid cancer, bladder cancer, plasmacytoma or brain cancer or wherein said autoimmune condition is ankylosing spondylitis, acute anterior uveitis, Goodpasture's syndrome, Multiple sclerosis, Graves' disease, Myasthenia gravis, Systemic lupus erythematosus, Insulin-dependent diabetes mellitus, Rheumatoid arthritis, Pemphigus vulgaris, Hashimoto's thyroiditis or autoimmune Hepatitis.

39. Use of the polynucleotide of claim 27 or the vector of claim 28 for the preparation of a composition for gene therapy.

40. A method for the treatment of cancer, infections or autoimmune conditions comprising introducing the polypeptide of any one of claims 1 to 26, the polynucleotide of claim 27 or the vector of claim 28 or the composition of claim 35 into a mammal affected by said malignancies or diseases.

41. A method for delaying a pathological condition comprising introducing the polypeptide of any one of claims 1 to 26, the polynucleotide of claim 27 or the vector of claim 28 or the composition of claim 35 into a mammal affected by said pathological condition.

42. The method of claim 40 or 41 wherein said mammal is a human.

43. A kit comprising the multifunctional polypeptide of any one of claims 1 to 26, the polynucleotide of claim 27, the vector of claim 28, the cell of claim 29 or the composition of any one of claims 31 to 36.


SEQUENCE LISTING (92 sequences)

SEQ ID NO: 1 - DNA, 429 bp, Homo sapiens
ttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtgtccgggcatcatcaccatcatcat

SEQ ID NO: 2 - protein, 143 aa, Homo sapiens
LFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSGHHHHHH

SEQ ID NO: 3 - protein, 133 aa, Homo sapiens
NQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 4 - protein, 114 aa, Homo sapiens
WICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 5 - protein, 30 aa, Homo sapiens
QTTPGERSSLPAFYPGTSGSCSGCGSLSLP

SEQ ID NO: 6 - protein, 21 aa, Homo sapiens
QTTPGERSSLPAFYPGTSGSC

SEQ ID NO: 7 - DNA, 321 bp, Artificial Sequence
  Description of Artificial Sequence Synthetic Anti-NKG2D hybridoma 11B2D10 variable light chain
gacattcagctgacccagtctccagcctccctatctgcatctgtgggagaaactgtcaccatcacatgtcgagcaagtgggaatattcacaattatttagcttggtatcagcagaaacagggaaaatctcctcaggtcctggtctataatgcaaaaaccttagcagatggtgtgccatcaaggttcagtggcagtggatcaggaacacaatattctctcaagatcaacagcctgcagcctgaagattttgggagttattactgtcaacatttttggagtactacgtggacgttcggtggagggaccaagctggagatcaaa

SEQ ID NO: 8 - protein, 107 aa, Artificial Sequence
  Description of Artificial Sequence Synthetic Anti-NKG2D hybridoma 11B2D10 variable light chain
DIQLTQSPASLSASVGETVTITCRASGNIHNYLAWYQQKQGKSPQVLVYNAKTLADGVPSRFSGSGSGTQYSLKINSLQPEDFGSYYCQHFWSTTWTFGGGTKLEIK

SEQ ID NO: 9 - protein, 11 aa, Artificial Sequence
  Description of Artificial Sequence Synthetic Anti-NKG2D hybridoma 11B2D10 variable light chain CDR1
RASGNIHNYLA

SEQ ID NO: 10 - protein, 7 aa, Artificial Sequence
  Description of Artificial Sequence Synthetic Anti-NKG2D hybridoma 11B2D10 variable light chain CDR2
NAKTLAD

SEQ ID NO: 11 - protein, 9 aa, Artificial Sequence
  Description of Artificial Sequence Synthetic Anti-NKG2D hybridoma 11B2D10 variable light chain CDR3
QHFWSTTWT

SEQ ID NO: 12 - DNA, 351 bp, Artificial Sequence
  Description of Artificial Sequence Synthetic Anti-NKG2D hybridoma 11B2D10 variable heavy chain
caggtccaactgcagcagtctggacctgagctggtgaggcctggggcttcagtgaagctgtcctgcaaggcttctggctacacgttcaccagctactggatgaactgggttcagcagaggcctgagcaaggccttgagtggattggaaggattgatccttacgatagtgaaactcactacaatcaaaagttcaaggacaaggccatattgactgtagacaaatccgccagcacagcctacatgcaactcagcagcctgacatctgaggactctgcggtctattactgtgcaaaaatgggtgattactcctttgactactggggccaagggaccacggtcaccgtctcctca

SEQ ID NO: 13 - protein, 117 aa, Artificial Sequence
  Description of Artificial Sequence Synthetic Anti-NKG2D hybridoma 11B2D10 variable heavy chain
QVQLQQSGPELVRPGASVKLSCKASGYTFTSYWMNWVQQRPEQGLEWIGRIDPYDSETHYNQKFKDKAILTVDKSASTAYMQLSSLTSEDSAVYYCAKMGDYSFDYWGQGTTVTVSS

SEQ ID NO: 14 - protein, 10 aa, Artificial Sequence
  Description of Artificial Sequence Synthetic Anti-NKG2D hybridoma 11B2D10 variable heavy chain CDR1
GYTFTSYWMN

SEQ ID NO: 15 - protein, 17 aa, Artificial Sequence
  Description of Artificial Sequence Synthetic Anti-NKG2D hybridoma 11B2D10 variable heavy chain CDR2
RIDPYDSETHYNQKFKD

SEQ ID NO: 16 - protein, 8 aa, Artificial Sequence
  Description of Artificial Sequence Synthetic Anti-NKG2D hybridoma 11B2D10 variable heavy chain CDR3
MGDYSFDY

SEQ ID NO: 17 - DNA, 318 bp, Artificial Sequence
  Description of Artificial Sequence Synthetic Anti-NKG2D hybridoma 6H7E7 variable light chain
gacattcagctgacccagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggaatagtaacccgctcacgttcggtgctgggaccaagctggagatcaaa

SEQ ID NO: 18 - protein, 106 aa, Artificial Sequence
  Description of Artificial Sequence Synthetic Anti-NKG2D hybridoma 6H7E7 variable light chain
DIQLTQSPAILSASPGEKVTMTCRASSSVSYMHWYQQKPGSSPKPWIYATSNLASGVPARFSGSGSGTSYSLTISRVEAEDAATYYCQQWNSNPLTFGAGTKLEIK

SEQ ID NO: 19 - protein, 10 aa, Artificial Sequence
  Description of Artificial Sequence Synthetic Anti-NKG2D hybridoma 6H7E7 variable light chain CDR1
RASSSVSYMH

SEQ ID NO: 20 - protein, 7 aa, Artificial Sequence
  Description of Artificial Sequence Synthetic Anti-NKG2D hybridoma 6H7E7 variable light chain CDR2
ATSNLAS

SEQ ID NO: 21 - protein, 9 aa, Artificial Sequence
  Description of Artificial Sequence Synthetic Anti-NKG2D hybridoma 6H7E7 variable light chain CDR3
QQWNSNPLT

SEQ ID NO: 22 - DNA, 357 bp, Artificial Sequence
  Description of Artificial Sequence Synthetic Anti-NKG2D hybridoma 6H7E7 variable heavy chain
caggtgcagctgcaggagtcaggacctggcctggtggcgccctcacagagcctgtccatcacttgcactgtctctgggttttcattaaccagctatggtgtacactggattcgccagcctccaggaaagggtctggagtggctgggagtaatatgggctggtggaagcacaaattataattcggctctcatgtccagactgagcatcagcaaagacaactccaagagccaagttttcttaaaaatgaatagtctgcaaattgatgacacagccatgtactactgtgccagaggggggtacgagggggcggcctggtttggttactggggccaagggaccacggtcaccgtctcctca

SEQ ID NO: 23 - protein, 119 aa, Artificial Sequence
  Description of Artificial Sequence Synthetic Anti-NKG2D hybridoma 6H7E7 variable heavy chain
QVQLQESGPGLVAPSQSLSITCTVSGFSLTSYGVHWIRQPPGKGLEWLGVIWAGGSTNYNSALMSRLSISKDNSKSQVFLKMNSLQIDDTAMYYCARGGYEGAAWFGYWGQGTTVTVSS

SEQ ID NO: 24 - protein, 10 aa, Artificial Sequence
  Description of Artificial Sequence Synthetic Anti-NKG2D hybridoma 6H7E7 variable heavy chain CDR1
GFSLTSYGVH

SEQ ID NO: 25 - protein, 16 aa, Artificial Sequence
  Description of Artificial Sequence Synthetic Anti-NKG2D hybridoma 6H7E7 variable heavy chain CDR2
VIWAGGSTNYNSALMS

SEQ ID NO: 26 - protein, 11 aa, Artificial Sequence
  Description of Artificial Sequence Synthetic Anti-NKG2D hybridoma 6H7E7 variable heavy chain CDR3
GGYEGAAWFGY

SEQ ID NO: 27 - DNA, 321 bp, Artificial Sequence
  Description of Artificial Sequence Synthetic Anti-NKG2D hybridoma 8G7C10 variable light chain
gacattcagctgacccagtctccagccatcctgtctgtgagtccaggagaaagagtcagtttctcctgcagggccagtcagaccattggcacaagcattcactggtatcagcaaagaacaaatggttctccaaggcttctcataaagtatgcttctgagtctatctctgggatcccttccaggtttagtggcagtggatcagggacagattttactcttagcatcaacggtgtggagtctgaagatattgcagattattactgtcaacaaagtaatacctggccactcacgttcggtgctgggaccaagctggagatcaaa

SEQ ID NO: 28 - protein, 107 aa, Artificial Sequence
  Description of Artificial Sequence Synthetic Anti-NKG2D hybridoma 8G7C10 variable light chain
DIQLTQSPAILSVSPGERVSFSCRASQTIGTSIHWYQQRTNGSPRLLIKYASESISGIPSRFSGSGSGTDFTLSINGVESEDIADYYCQQSNTWPLTFGAGTKLEIK

SEQ ID NO: 29 - protein, 11 aa, Artificial Sequence
  Description of Artificial Sequence Synthetic Anti-NKG2D hybridoma 8G7C10 variable light chain CDR1
RASQTIGTSIH

SEQ ID NO: 30 - protein, 7 aa, Artificial Sequence
  Description of Artificial Sequence Synthetic Anti-NKG2D hybridoma 8G7C10 variable light chain CDR2
YASESIS

SEQ ID NO: 31 - protein, 9 aa, Artificial Sequence
  Description of Artificial Sequence Synthetic Anti-NKG2D hybridoma 8G7C10 variable light chain CDR3
QQSNTWPLT

SEQ ID NO: 32 - DNA, 360 bp, Artificial Sequence
  Description of Artificial Sequence Synthetic Anti-NKG2D hybridoma 8G7C10 variable heavy chain
caggtgcagctgcagcagtcaggacctggcctagtgcagccctcacagagcctgtccatcacctgcacagtctctggtttctcattaactatctatggtgtacactgggttcgccagtctccaggaaagggtctggagtggctgggagtgatatggagtggcggaagcacagactataatgcagctttcatatccagactgagcatcagcaaggacaattccaagcgccaagttttctttaaaatgagcagtctgcaagctaatgacacagccatatattactgttccagaaagtcccatgatggttactacggagtaatggactactggggccaagggaccacggtcaccgtctcctca

SEQ ID NO: 33 - protein, 120 aa, Artificial Sequence
  Description of Artificial Sequence Synthetic Anti-NKG2D hybridoma 8G7C10 variable heavy chain
QVQLQQSGPGLVQPSQSLSITCTVSGFSLTIYGVHWVRQSPGKGLEWLGVIWSGGSTDYNAAFISRLSISKDNSKRQVFFKMSSLQANDTAIYYCSRKSHDGYYGVMDYWGQGTTVTVSS

SEQ ID NO: 34 - protein, 10 aa, Artificial Sequence
  Description of Artificial Sequence Synthetic Anti-NKG2D hybridoma 8G7C10 variable heavy chain CDR1
GFSLTIYGVH

SEQ ID NO: 35 - protein, 16 aa, Artificial Sequence
  Description of Artificial Sequence Synthetic Anti-NKG2D hybridoma 8G7C10 variable heavy chain CDR2
VIWSGGSTDYNAAFIS

SEQ ID NO: 36 - protein, 12 aa, Artificial Sequence
  Description of Artificial Sequence Synthetic Anti-NKG2D hybridoma 8G7C10 variable heavy chain CDR3
KSHDGYYGVMDY

SEQ ID NO: 37 - DNA, 321 bp, Artificial Sequence
  Description of Artificial Sequence Synthetic Anti-NKG2D hybridoma 6E5A7 variable light chain
gacattcagctgacccagtctccagccatcctgtctgtgagtccaggagaaagagtcagtttctcctgcagggccagtcagagcattggcacaagcattcactggtatcagcaaagaacaaatggttctccaaggcttctcataaagtatgcttctgagtctatctctgggatcccttccaggtttagtggcagtggatcagggacagattttactcttagcatcaacggtgtggagtctgaagatattgcagattattactgtcaacaaagtaatacctggccactcacgttcggtgctgggaccaagctggagatcaaa

SEQ ID NO: 38 - protein, 107 aa, Artificial Sequence
  Description of Artificial Sequence Synthetic Anti-NKG2D hybridoma 6E5A7 variable light chain
DIQLTQSPAILSVSPGERVSFSCRASQSIGTSIHWYQQRTNGSPRLLIKYASESISGIPSRFSGSGSGTDFTLSINGVESEDIADYYCQQSNTWPLTFGAGTKLEIK

SEQ ID NO: 39 - protein, 11 aa, Artificial Sequence
  Description of Artificial Sequence Synthetic Anti-NKG2D hybridoma 6E5A7 variable light chain CDR1
RASQSIGTSIH

SEQ ID NO: 40 - protein, 7 aa, Artificial Sequence
  Description of Artificial Sequence Synthetic Anti-NKG2D hybridoma 6E5A7 variable light chain CDR2
YASESIS

SEQ ID NO: 41 - protein, 9 aa, Artificial Sequence
  Description of Artificial Sequence Synthetic Anti-NKG2D hybridoma 6E5A7 variable light chain CDR3
QQSNTWPLT

SEQ ID NO: 42 - DNA, 360 bp, Artificial Sequence; identical to SEQ ID NO: 32
  Description of Artificial Sequence Synthetic Anti-NKG2D hybridoma 6E5A7 variable heavy chain

SEQ ID NO: 43 - protein, 120 aa, Artificial Sequence; identical to SEQ ID NO: 33
  Description of Artificial Sequence Synthetic Anti-NKG2D hybridoma 6E5A7 variable heavy chain

SEQ ID NO: 44 - protein, 10 aa, Artificial Sequence
  Description of Artificial Sequence Synthetic Anti-NKG2D hybridoma 6E5A7 variable heavy chain CDR1
GFSLTIYGVH

SEQ ID NO: 45 - protein, 16 aa, Artificial Sequence
  Description of Artificial Sequence Synthetic Anti-NKG2D hybridoma 6E5A7 variable heavy chain CDR2
VIWSGGSTDYNAAFIS

SEQ ID NO: 46 - protein, 12 aa, Artificial Sequence
  Description of Artificial Sequence Synthetic Anti-NKG2D hybridoma 6E5A7 variable heavy chain CDR3
KSHDGYYGVMDY

SEQ ID NO: 47 - protein, 507 aa, Artificial Sequence
  Description of Artificial Sequence Synthetic 11B2D10x4- 7 bispecific single chain Fv
DIQLTQSPASLSASVGETVTITCRASGNIHNYLAWYQQKQGKSPQVLVYNAKTLADGVPSRFSGSGSGTQYSLKINSLQPEDFGSYYCQHFWSTTWTFGGGTKLEIKGGGGSGGGGSGGGGSQVQLQQSGPELVRPGASVKLSCKASGYTFTSYWMNWVQQRPEQGLEWIGRIDPYDSETHYNQKFKDKAILTVDKSASTAYMQLSSLTSEDSAVYYCAKMGDYSFDYWGQGTTVTVSSGGGGSEVQLLEQSGAELARPGASVKLSCKASGYTFTNYGLSWVKQRPGQVLEWIGEVYPRIGNAYYNEKFKGKATLTADKSSSTASMELRSLTSEDSAVYFCARRGSYDTNYDWYFDVWGQGTTVTVSSGGGGSGGGGSGGGGSELVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKRTTSHHHHHHTS

SEQ ID NO: 48 - protein, 510 aa, Artificial Sequence
  Description of Artificial Sequence Synthetic 8G7C10x4-7 bispecific single chain Fv
DIQLTQSPAILSVSPGERVSFSCRASQTIGTSIHWYQQRTNGSPRLLIKYASESISGIPSRFSGSGSGTDFTLSINGVESEDIADYYCQQSNTWPLTFGAGTKLEIKGGGGSGGGGSGGGGSQVQLQQSGPGLVQPSQSLSITCTVSGFSLTIYGVHWVRQSPGKGLEWLGVIWSGGSTDYNAAFISRLSISKDNSKRQVFFKMSSLQANDTAIYYCSRKSHDGYYGVMDYWGQGTTVTVSSGGGGSEVQLLEQSGAELARPGASVKLSCKASGYTFTNYGLSWVKQRPGQVLEWIGEVYPRIGNAYYNEKFKGKATLTADKSSSTASMELRSLTSEDSAVYFCARRGSYDTNYDWYFDVWGQGTTVTVSSGGGGSGGGGSGGGGSELVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKRTTSHHHHHHTS

SEQ ID NO: 49 - protein, 510 aa, Artificial Sequence
  Description of Artificial Sequence Synthetic 6E5A7x4-7 bispecific single chain Fv
DIQLTQSPAILSVSPGERVSFSCRASQSIGTSIHWYQQRTNGSPRLLIKYASESISGIPSRFSGSGSGTDFTLSINGVESEDIADYYCQQSNTWPLTFGAGTKLEIKGGGGSGGGGSGGGGSQVQLQQSGPGLVQPSQSLSITCTVSGFSLTIYGVHWVRQSPGKGLEWLGVIWSGGSTDYNAAFISRLSISKDNSKRQVFFKMSSLQANDTAIYYCSRKSHDGYYGVMDYWGQGTTVTVSSGGGGSEVQLLEQSGAELARPGASVKLSCKASGYTFTNYGLSWVKQRPGQVLEWIGEVYPRIGNAYYNEKFKGKATLTADKSSSTASMELRSLTSEDSAVYFCARRGSYDTNYDWYFDVWGQGTTVTVSSGGGGSGGGGSGGGGSELVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKRTTSHHHHHHTS

SEQ ID NO: 50 - DNA, 206 bp, Homo sapiens
actagttccggaaccccgctgggtgacaccacccacacctctggaaaaccactggatggagaatatttcacccttcagatccgtgggcgtgagcgcttcgagatgttccgagagctgaatgaggccttggaactcaaggatgcccaggctgggaaggagccaggggggagcgactacaaggatgacgatgacaagtaagcggccgc

SEQ ID NO: 51 - protein, 65 aa, Homo sapiens
TSSGTPLGDTTHTSGKPLDGEYFTLQIRGRERFEMFRELNEALELKDAQAGKEPGGSDYKDDDDK

SEQ ID NO: 52 - DNA, 777 bp, Artificial Sequence
  Description of Artificial Sequence Synthetic P4-2 single chain Fv
gaggtgcagctgctcgaggagtctggaggaggcttggtacagcctgggggttctctgagactctcctgtgcaacttctgggttcaccttcactgattactacatgagctgggtccgccagcctccaggaaaggcacttgagtggttgggttttattagaaacaaagctaatggttacacaacagagtacagtgcatctgtgaagggtcggttcaccatctccagagataattcccaaagcatcctctatcttcaaatgaacaccctgagagctgaggacagtcccacttattactgtgcaagagataagacagacttcgatgtctggggccaagggaccacggtcaccgtctcctcaggtggtggtggttctggcggcggcggctccggtggtggtggttctgagctcgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagcttgagatcaaacgtacgactagttccgggcatcatcaccatcatcat

SEQ ID NO: 53 - protein, 259 aa, Artificial Sequence
  Description of Artificial Sequence Synthetic P4-2 single chain Fv
EVQLLEESGGGLVQPGGSLRLSCATSGFTFTDYYMSWVRQPPGKALEWLGFIRNKANGYTTEYSASVKGRFTISRDNSQSILYLQMNTLRAEDSPTYYCARDKTDFDVWGQGTTVTVSSGGGGSGGGGSGGGGSELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKRTTSSGHHHHHH

SEQ ID NO: 54 - DNA, 756 bp, Artificial Sequence
  Description of Artificial Sequence Synthetic P4-3 single chain Fv
gaggtgcagctgctcgagtctggaggtggcctggtgcagcctggaggatccctgaaactctcctgtgcagcctctggattcgattttagtagatactggatgagttgggtccggcaggctccagggaaagggctagaatggattggagaaattaatccagatagcagtacgataaactatacgccatctctaaaggataaattcatcatctccagagacaacgccaaaaatacgctgtacctgcaaatgagcaaagtgagatctgaggacacagccctttattactgtgcaagaggggcggtagtagctccctttgactactggggccaagggaccacggtcaccgtctcctcaggtggtggtggttctggcggcggcggctccggtggtggtggttctgagctcgtcatgacccagtctccatcctccttatctgcctctctgggagaaagagtcagtctcacttgtcgggcaagtcaggacattggtagtagcttaaactggcttcagcaggaaccagatggaactattaaacgcctgatctacgccacatccagtttagattctggtgtccccaaaaggttcagtggcagtaggtctgggtcagattattctctcaccatcagcagccttgagtctgaagattttgtagactattactgtctacaatatgctagttctccgtacacgttcggaggggggaccaagcttgagatcaaacgtacgactagttccgggcatcatcaccatcatcat

SEQ ID NO: 55 - protein, 252 aa, Artificial Sequence
  Description of Artificial Sequence Synthetic P4-3 single chain Fv
EVQLLESGGGLVQPGGSLKLSCAASGFDFSRYWMSWVRQAPGKGLEWIGEINPDSSTINYTPSLKDKFIISRDNAKNTLYLQMSKVRSEDTALYYCARGAVVAPFDYWGQGTTVTVSSGGGGSGGGGSGGGGSELVMTQSPSSLSASLGERVSLTCRASQDIGSSLNWLQQEPDGTIKRLIYATSSLDSGVPKRFSGSRSGSDYSLTISSLESEDFVDYYCLQYASSPYTFGGGTKLEIKRTTSSGHHHHHH

SEQ ID NO: 56 - DNA, 768 bp, Artificial Sequence
  Description of Artificial Sequence Synthetic P4-14 single chain Fv
gaggtgcagctgctcgagtctggaggtggcctggtgcagcctggaggatccctgaaactctcctgtgcagcctcaggattcgattttagtagatactggatgagttgggtccggcaggctccagggaaagggctagaatggattggagaaattaatccagatagcagtacgataaactatacgccatctctaaaggataaattcatcatctccagagacaacgccaaaaatacgctgtacctgcaaatgagcaaagtgagatctgaggacacagccctttattactgtgcaagacgcagctacggtagtagctacgactggtacttcgatgtctggggccaagggaccacggtcaccgtctcctcaggtggtggtggttctggcggcggcggctccggtggtggtggttctgagctccagatgacccagtctccagcctccctatctgcatctgtgggagaaactgtcaccatcacatgtcgagcaagtgagaatatttacagttatttagcatggtatcagcagaaacagggaaaatctcctcagctcctggtctataatgcaaaaaccttagcagaaggtgtgccatcaaggttcagtagcagtggatcaggcacacagttttctctgaagatcaacagcctgcagcctgaagattttgggagttattactgtcaacatcattatggtactccgctcacgttcggtgctgggaccaagcttgagatcaaacgtacgactagttccgggcatcatcaccatcatcat

SEQ ID NO: 57 - protein, 256 aa, Artificial Sequence
  Description of Artificial Sequence Synthetic P4-14 single chain Fv
EVQLLESGGGLVQPGGSLKLSCAASGFDFSRYWMSWVRQAPGKGLEWIGEINPDSSTINYTPSLKDKFIISRDNAKNTLYLQMSKVRSEDTALYYCARRSYGSSYDWYFDVWGQGTTVTVSSGGGGSGGGGSGGGGSELQMTQSPASLSASVGETVTITCRASENIYSYLAWYQQKQGKSPQLLVYNAKTLAEGVPSRFSSSGSGTQFSLKINSLQPEDFGSYYCQHHYGTPLTFGAGTKLEIKRTTSSGHHHHHH

SEQ ID NO: 58 - DNA, 774 bp, Artificial Sequence
  Description of Artificial Sequence Synthetic P4-15 single chain Fv
gaggtgcagctgctcgagcagtctggagctgagctgatgaagcctggggcctcagtgaagatatcctgcaaggctactggctacacattcagtagctactggatagagtgggtaaagcagaggcctggacatggccttgagtggattggagagattttacctggaagtggtagtactaactacaatgagaagttcaagggcaaggccacattcactgcagatacatcctccaacacagcctacatgcaactcagcagcctgacatctgaggactctgccgtctattactgtgcaagaggattacgacgttggtttgcttactggggccaagggaccacggtcaccgtctcctcaggtggtggtggttctggcggcggcggctccggtggtggtggttctgagctcgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagcttgagatcaaacgtacgactagttccgggcatcatcaccatcatcat

SEQ ID NO: 59 - protein, 258 aa, Artificial Sequence
  Description of Artificial Sequence Synthetic P4-15 single chain Fv
EVQLLEQSGAELMKPGASVKISCKATGYTFSSYWIEWVKQRPGHGLEWIGEILPGSGSTNYNEKFKGKATFTADTSSNTAYMQLSSLTSEDSAVYYCARGLRRWFAYWGQGTTVTVSSGGGGSGGGGSGGGGSELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKRTTSSGHHHHHH

SEQ ID NO: 60 - DNA, 768 bp, Artificial Sequence
  Description of Artificial Sequence Synthetic P5-2 single chain Fv
gaggtgcagctgctcgaggagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtaccaggctcccctacggctttgctatggactactggggccaagggaccacggtcaccgtctcctcaggtggtggtggttctggcggcggcggctccggtggtggtggttctgagctcgtgctcacccagtctccaaccaccatggctgcatctcccggggagaagatcactatcacctgcagtgccagctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtataccgctcacgttcggtgctgggaccaagcttgagatcaaacgtacgactagttccgggcatcatcaccatcatcat

SEQ ID NO: 61 - protein, 256 aa, Artificial Sequence
  Description of Artificial Sequence Synthetic P5-2 single chain Fv
EVQLLEESGGGLVQPGGSMKLSCVASGFTFSNYWMNWVRQSPEKGLEWVAEIRLKSNNYATHYAESVKGRFTISRDDSKSSVYLQMNNLRAEDTGIYYCTRLPYGFAMDYWGQGTTVTVSSGGGGSGGGGSGGGGSELVLTQSPTTMAASPGEKITITCSASSSISSNYLHWYQQKPGFSPKLLIYRTSNLASGVPARFSGSGSGTSYSLTIGTMEAEDVATYYCQQGSSIPLTFGAGTKLEIKRTTSSGHHHHHH

SEQ ID NO: 62 - DNA, 759 bp, Artificial Sequence
  Description of Artificial Sequence Synthetic P5-3 single chain Fv
gaggtgcagctgctcgaggagtcaggacctggcctggtggcgccctcacagagcctgtccatcacttgcactgtctctgggttttcattaaccagctatggtgtacactgggttcgccagcctccaggaaagggtctggagtggctgggagtaatatgggctggtggaagcacaaattataattcggctctcatgtccagactgagcatcagcaaagacaactccaagagccaagttttcttaaaaatgaacagtctgcaaactgatgacacagccatgtactactgtgccagagatcggtactacgtgggtgctatggactactggggccaagggaccacggtcaccgtctcctcaggtggtggtggttctggcggcggcggctccggtggtggtggttctgagctccagatgacccagtctccagcatccctgtccatggctataggagaaaaagtcaccatcagatgcataaccagcactgatattgatgatgatatgaactggtaccagcagaagccaggggaacctcctaagctccttatttcagaaggcaatactcttcgtcctggagtcccatcccgattctccagcagtggctatggtacagattttgtttttacaattgaaaacatgctctcagaagatgttgcagattactactgtttgcaaagtgataacttgccgtacacgttcggaggggggaccaagcttgagatcaaacgtacgactagttccgggcatcatcaccatcatcat

SEQ ID NO: 63 - protein, 253 aa, Artificial Sequence
  Description of Artificial Sequence Synthetic P5-3 single chain Fv
EVQLLEESGPGLVAPSQSLSITCTVSGFSLTSYGVHWVRQPPGKGLEWLGVIWAGGSTNYNSALMSRLSISKDNSKSQVFLKMNSLQTDDTAMYYCARDRYYVGAMDYWGQGTTVTVSSGGGGSGGGGSGGGGSELQMTQSPASLSMAIGEKVTIRCITSTDIDDDMNWYQQKPGEPPKLLISEGNTLRPGVPSRFSSSGYGTDFVFTIENMLSEDVADYYCLQSDNLPYTFGGGTKLEIKRTTSSGHHHHHH

SEQ ID NO: 64 - DNA, 753 bp, Artificial Sequence
  Description of Artificial Sequence Synthetic P5-9 single chain Fv
gaggtgcagctgctcgagtctggaggtggcctggtgcagcctggaggatccctgaaactctcctgtgcagcctcaggattcgattttagtagatactggatgagttgggtccggcaggctccagggaaagggctagaatggattggagaaattaatccagatagcagtacgataaactatacgccatctctaaaggataaattcatcatctccagagacaacgccaaaaatacgctgtacctgcaaatgagcaaagtgagatctgaggacacagccctttattactgtgcaagggaaactgggacggagtttgactactggggccaagggaccacggtcaccgtctcctcaggtggtggtggttctggcggcggcggctccggtggtggtggttctgagctcgtgatgacccagactccatcctccatgtatgcatcgctgggagagagagtcactatcacttgcaaggcgagtcaggacattaaaagctatttaagctggtaccagcagaaaccatggaaatctcctaagaccctgatctattatgcaacaagcttggcagatggggtcccatcaagattcagtggcagtggatctgggcaagattattctctaaccatcagcagcctggagtctgacgatacagcaacttattactgtctacagcatggtgagagcccgtacacgttcggaggggggaccaagcttgagatcaaacgtacgactagttccgggcatcatcaccatcatcat

SEQ ID NO: 65 - protein, 251 aa, Artificial Sequence
  Description of Artificial Sequence Synthetic P5-9 single chain Fv
EVQLLESGGGLVQPGGSLKLSCAASGFDFSRYWMSWVRQAPGKGLEWIGEINPDSSTINYTPSLKDKFIISRDNAKNTLYLQMSKVRSEDTALYYCARETGTEFDYWGQGTTVTVSSGGGGSGGGGSGGGGSELVMTQTPSSMYASLGERVTITCKASQDIKSYLSWYQQKPWKSPKTLIYYATSLADGVPSRFSGSGSGQDYSLTISSLESDDTATYYCLQHGESPYTFGGGTKLEIKRTTSSGHHHHHH

SEQ ID NO: 66 - DNA, 771 bp, Artificial Sequence
  Description of Artificial Sequence Synthetic P5-10 single chain Fv
gaggtgcagctgctcgagcagtctggagctgagcttgtgaggccaggggccttagtcaagttgtcctgcaaagcttctggcttcaacattaaagactactatatgcactgggtgaagcagaggcctgaacagggcctggagtggattggatggattgatcctgagaatggtaatactatatatgacccgaagttccagggcaaggccagtataacagcagacacatcctccaacacagcctacctgcagctcagcagcctgacatctgaggacactgccgcctattactgtgcttccttttattactacggtagtagctacaggtacttcgatgtctggggccaagggaccacggtcaccgtctcctcaggtggtggtggttctggcggcggcggctccggtggtggtggttctgagctcgtgatgacccagactccatcctccttatctgcctctctgggagaaagagtcagtctcacttgtcgggcaagtcaggacattggtagtagcttaaactggcttcagcaggaaccagatggaactattaaacgcctgatctacgccacatccagtttagattctggtgtccccaaaaggttcagtggcagtaggtctgggtcagattattctctcaccatcagcagccttgagtctgaagattttgtagactattactgtctacaatatgctagttctccgtacacgttcggaggggggaccaagcttgagatcaaacgtacgactagttccgggcatcatcaccatcatcat

SEQ ID NO: 67 - protein, 257 aa, Artificial Sequence
  Description of Artificial Sequence Synthetic P5-10 single chain Fv
EVQLLEQSGAELVRPGALVKLSCKASGFNIKDYYMHWVKQRPEQGLEWIGWIDPENGNTIYDPKFQGKASITADTSSNTAYLQLSSLTSEDTAAYYCASFYYYGSSYRYFDVWGQGTTVTVSSGGGGSGGGGSGGGGSELVMTQTPSSLSASLGERVSLTCRASQDIGSSLNWLQQEPDGTIKRLIYATSSLDSGVPKRFSGSRSGSDYSLTISSLESEDFVDYYCLQYASSPYTFGGGTKLEIKRTTSSGHHHHHH

SEQ ID NO: 68 - DNA, 765 bp, Artificial Sequence
  Description of Artificial Sequence Synthetic P5-11 single chain Fv
gaggtgcagctgctcgaggagtctggaggaggcttggtgcaacctggaggatccatgaaactctcctgtattgcctctggattcactttcagtaattcctggatgaactgggtccgccagtctccagagaaggggcttgagtgggttggtgaaattagattgaaatctaataattatgcaacacattatgcggagtctgtgaaagggaggttcaccatctcaagagatgattccaaaagtagtgtctacctacaaatgaacaacttaagagttgaagacactggcatttattactgtacgaaggtggactactggggccaagggaccacggtcaccgtctcctcaggtggtggtggttctggcggcggcggctccggtggtggtggttctgagctcgtgatgacacagtctccatcctccctggctatgtcagtaggacagaaggtcactatgagctgcaagtccagtcagagccttttaaatagtagcaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagcttgagatcaaacgtacgactagttccgggcatcatcaccatcatcat

SEQ ID NO: 69 - protein, 255 aa, Artificial Sequence
  Description of Artificial Sequence Synthetic P5-11 single chain Fv
EVQLLEESGGGLVQPGGSMKLSCIASGFTFSNSWMNWVRQSPEKGLEWVGEIRLKSNNYATHYAESVKGRFTISRDDSKSSVYLQMNNLRVEDTGIYYCTKVDYWGQGTTVTVSSGGGGSGGGGSGGGGSELVMTQSPSSLAMSVGQKVTMSCKSSQSLLNSSNQKNYLTWYQQKPGQPPKLLIYWASTRESGVPDRFTGSGSGTDFTLTISSVQAEDLAVYYCQNDYSYPLTFGAGTKLEIKRTTSSGHHHHHH

SEQ ID NO: 70 - DNA, 774 bp, Artificial Sequence
  Description of Artificial Sequence Synthetic P5-23 single chain Fv
gaggtgcagctgctcgagtctggaggtggcctggtgcagcctggaggatccctgaaactctcctgtgcagcctcaggattcgattttagtagatactggatgagttgggtccggcaggctccagggaaagggctagaatggattggagaaattaatccagatagcagtacgataaactatacgccatctctaaaggatagattcatcatctccagagacaacgccaaaaatacgctgtacctgcaaatgagcaaagtgaggtctgaggacacagccctttattactgtgcaagattggggcaatgggggtactttgactactggggccaagggaccacggtcaccgtctcctcaggtggtggtggttctggcggcggcggctccggtggtggtggttctgagctcgtgatgacacagtctccatcctccctgactgtgacagcaggagagagggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatcctctcacgttcggtgctgggaccaagcttgagatcaaacgtacgactagttccgggcatcatcaccatcatcat

SEQ ID NO: 71 - protein, 258 aa, Artificial Sequence
  Description of Artificial Sequence Synthetic P5-23 single chain Fv
EVQLLESGGGLVQPGGSLKLSCAASGFDFSRYWMSWVRQAPGKGLEWIGEINPDSSTINYTPSLKDRFIISRDNAKNTLYLQMSKVRSEDTALYYCARLGQWGYFDYWGQGTTVTVSSGGGGSGGGGSGGGGSELVMTQSPSSLTVTAGERVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKRTTSSGHHHHHH

SEQ ID NO: 72 - DNA, 1497 bp, Artificial Sequence
  Description of Artificial Sequence Synthetic 3B10xP4-3 bispecific single chain Fv
gatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttcctcggacgttcggtggaggcaccaagctggaaatcaaaggtggtggtggttctggcggcggcggctccggtggtggtggttctcaggtgcaactgcagcagtcagggcctgagctgaagaagcctggagagacagtcaagatctcctgcaaggcttctgggtataccttcacaaactatggaatgaactgggtgaagcaggctccaggaaagggtttcaagtggatgggctggataaacacctacactggagagccaacatatggtgatgacttcaagggacggtttgccttctctttggaaacctctgccagcactgcctatttgcagatcaacaacctcaaaaatgaggacacggctacatatttctgtgcaagattcacctcccctgactactggggccaagggaccacggtcaccgtctcctccggaggtggtggatccgaggtgcagctgctcgagtctggaggtggcctggtgcagcctggaggatccctgaaactctcctgtgcagcctctggattcgattttagtagatactggatgagttgggtccggcaggctccagggaaagggctagaatggattggagaaattaatccagatagcagtacgataaactatacgccatctctaaaggataaattcatcatctccagagacaacgccaaaaatacgctgtacctgcaaatgagcaaagtgagatctgaggacacagccctttattactgtgcaagaggggcggtagtagctccctttgactactggggccaagggaccacggtcaccgtctcctcaggtggtggtggttctggcggcggcggctccggtggtggtggttctgagctcgtcatgacccagtctccatcctccttatctgcctctctgggagaaagagtcagtctcacttgtcgggcaagtcaggacattggtagtagcttaaactggcttcagcaggaaccagatggaactattaaacgcctgatctacgccacatccagtttagattctggtgtccccaaaaggttcagtggcagtaggtctgggtcagattattctctcaccatcagcagccttgagtctgaagattttgtagactattactgtctacaatatgctagttctccgtacacgttcggaggggggaccaagcttgagatcaaacgtacgactagttccgggcatcatcaccatcatcat

SEQ ID NO: 73 - protein, 499 aa, Artificial Sequence
  Description of Artificial Sequence Synthetic 3B10xP4-3 bispecific single chain Fv
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPRTFGGGTKLEIKGGGGSGGGGSGGGGSQVQLQQSGPELKKPGETVKISCKASGYTFTNYGMNWVKQAPGKGFKWMGWINTYTGEPTYGDDFKGRFAFSLETSASTAYLQINNLKNEDTATYFCARFTSPDYWGQGTTVTVSSGGGGSEVQLLESGGGLVQPGGSLKLSCAASGFDFSRYWMSWVRQAPGKGLEWIGEINPDSSTINYTPSLKDKFIISRDNAKNTLYLQMSKVRSEDTALYYCARGAVVAPFDYWGQGTTVTVSSGGGGSGGGGSGGGGSELVMTQSPSSLSASLGERVSLTCRASQDIGSSLNWLQQEPDGTIKRLIYATSSLDSGVPKRFSGSRSGSDYSLTISSLESEDFVDYYCLQYASSPYTFGGGTKLEIKRTTSSGHHHHHH

SEQ ID NO: 74 - DNA, 1509 bp, Artificial Sequence
  Description of Artificial Sequence Synthetic 3B10xP4-14 bispecific single chain Fv
gatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttcctcggacgttcggtggaggcaccaagctggaaatcaaaggtggtggtggttctggcggcggcggctccggtggtggtggttctcaggtgcaactgcagcagtcagggcctgagctgaagaagcctggagagacagtcaagatctcctgcaaggcttctgggtataccttcacaaactatggaatgaactgggtgaagcaggctccaggaaagggtttcaagtggatgggctggataaacacctacactggagagccaacatatggtgatgacttcaagggacggtttgccttctctttggaaacctctgccagcactgcctatttgcagatcaacaacctcaaaaatgaggacacggctacatatttctgtgcaagattcacctcccctgactactggggccaagggaccacggtcaccgtctcctccggaggtggtggatccgaggtgcagctgctcgagtctggaggtggcctggtgcagcctggaggatccctgaaactctcctgtgcagcctcaggattcgattttagtagatactggatgagttgggtccggcaggctccagggaaagggctagaatggattggagaaattaatccagatagcagtacgataaactatacgccatctctaaaggataaattcatcatctccagagacaacgccaaaaatacgctgtacctgcaaatgagcaaagtgagatctgaggacacagccctttattactgtgcaagacgcagctacggtagtagctacgactggtacttcgatgtctggggccaagggaccacggtcaccgtctcctcaggtggtggtggttctggcggcggcggctccggtggtggtggttctgagctccagatgacccagtctccagcctccctatctgcatctgtgggagaaactgtcaccatcacatgtcgagcaagtgagaatatttacagttatttagcatggtatcagcagaaacagggaaaatctcctcagctcctggtctataatgcaaaaaccttagcagaaggtgtgccatcaaggttcagtagcagtggatcaggcacacagttttctctgaagatcaacagcctgcagcctgaagattttgggagttattactgtcaacatcattatggtactccgctcacgttcggtgctgggaccaagcttgagatcaaacgtacgactagttccgggcatcatcaccatcatcat

SEQ ID NO: 75 - protein, 503 aa, Artificial Sequence
  Description of Artificial Sequence Synthetic 3B10xP4-14 bispecific single chain Fv
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPRTFGGGTKLEIKGGGGSGGGGSGGGGSQVQLQQSGPELKKPGETVKISCKASGYTFTNYGMNWVKQAPGKGFKWMGWINTYTGEPTYGDDFKGRFAFSLETSASTAYLQINNLKNEDTATYFCARFTSPDYWGQGTTVTVSSGGGGSEVQLLESGGGLVQPGGSLKLSCAASGFDFSRYWMSWVRQAPGKGLEWIGEINPDSSTINYTPSLKDKFIISRDNAKNTLYLQMSKVRSEDTALYYCARRSYGSSYDWYFDVWGQGTTVTVSSGGGGSGGGGSGGGGSELQMTQSPASLSASVGETVTITCRASENIYSYLAWYQQKQGKSPQLLVYNAKTLAEGVPSRFSSSGSGTQFSLKINSLQPEDFGSYYCQHHYGTPLTFGAGTKLEIKRTTSSGHHHHHH

SEQ ID NO: 76 - DNA, 1509 bp, Artificial Sequence
  Description of Artificial Sequence Synthetic 3B10xP5-2 bispecific single chain Fv
gatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttcctcggacgttcggtggaggcaccaagctggaaatcaaaggtggtggtggttctggcggcggcggctccggtggtggtggttctcaggtgcaactgcagcagtcagggcctgagctgaagaagcctggagagacagtcaagatctcctgcaaggcttctgggtataccttcacaaactatggaatgaactgggtgaagcaggctccaggaaagggtttcaagtggatgggctggataaacacctacactggagagccaacatatggtgatgacttcaagggacggtttgccttctctttggaaacctctgccagcactgcctatttgcagatcaacaacctcaaaaatgaggacacggctacatatttctgtgcaagattcacctcccctgactactggggccaagggaccacggtcaccgtctcctccggaggtggtggatccgaggtgcagctgctcgaggagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtaccaggctcccctacggctttgctatggactactggggccaagggaccacggtcaccgtctcctcaggtggtggtggttctggcggcggcggctccggtggtggtggttctgagctcgtgctcacccagtctccaaccaccatggctgcatctcccggggagaagatcactatcacctgcagtgccagctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtataccgctcacgttcggtgctgggaccaagcttgagatcaaacgtacgactagttccgggcatcatcaccatcatcat

SEQ ID NO: 77 - protein, 503 aa, Artificial Sequence
  Description of Artificial Sequence Synthetic 3B10xP5-2 bispecific single chain Fv
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPRTFGGGTKLEIKGGGGSGGGGSGGGGSQVQLQQSGPELKKPGETVKISCKASGYTFTNYGMNWVKQAPGKGFKWMGWINTYTGEPTYGDDFKGRFAFSLETSASTAYLQINNLKNEDTATYFCARFTSPDYWGQGTTVTVSSGGGGSEVQLLEESGGGLVQPGGSMKLSCVASGFTFSNYWMNWVRQSPEKGLEWVAEIRLKSNNYATHYAESVKGRFTISRDDSKSSVYLQMNNLRAEDTGIYYCTRLPYGFAMDYWGQGTTVTVSSGGGGSGGGGSGGGGSELVLTQSPTTMAASPGEKITITCSASSSISSNYLHWYQQKPGFSPKLLIYRTSNLASGVPARFSGSGSGTSYSLTIGTMEAEDVATYYCQQGSSIPLTFGAGTKLEIKRTTSSGHHHHHH

SEQ ID NO: 78 - DNA, 1515 bp, Artificial Sequence
  Description of Artificial Sequence Synthetic 3B10xP5-23 bispecific single chain Fv
gatattgtgatgacgcaggctgcattctccaatccagtcactcttggaacatcagcttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttcctcggacgttcggtggaggcaccaagctggaaatcaaaggtggtggtggttctggcggcggcggctccggtggtggtggttctcaggtgcaactgcagcagtcagggcctgagctgaagaagcctggagagacagtcaagatctcctgcaaggcttctgggtataccttcacaaactatggaatgaactgggtgaagcaggctccaggaaagggtttcaagtggatgggctggataaacacctacactggagagccaacatatggtgatgacttcaagggacggtttgccttctctttggaaacctctgccagcactgcctatttgcagatcaacaacctcaaaaatgaggacacggctacatatttctgtgcaagattcacctcccctgactactggggccaagggaccacggtcaccgtctcctccggaggtggtggatccgaggtgcagctgctcgagtctggaggtggcctggtgcagcctggaggatccctgaaactctcctgtgcagcctcaggattcgattttagtagatactggatgagttgggtccggcaggctccagggaaagggctagaatggattggagaaattaatccagatagcagtacgataaactatacgccatctctaaaggatagattcatcatctccagagacaacgccaaaaatacgctgtacctgcaaatgagcaaagtgaggtctgaggacacagccctttattactgtgcaagattggggcaatgggggtactttgactactggggccaagggaccacggtcaccgtctcctcaggtggtggtggttctggcggcggcggctccggtggtggtggttctgagctcgtgatgacacagtctccatcctccctgactgtgacagcaggagagagggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatcctctcacgttcggtgctgggaccaagcttgagatcaaacgtacgactagttccgggcatcatcaccatcatcat

SEQ ID NO: 79 - protein, 505 aa, Artificial Sequence
  Description of Artificial Sequence Synthetic 3B10xP5-23 bispecific single chain Fv
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPRTFGGGTKLEIKGGGGSGGGGSGGGGSQVQLQQSGPELKKPGETVKISCKASGYTFTNYGMNWVKQAPGKGFKWMGWINTYTGEPTYGDDFKGRFAFSLETSASTAYLQINNLKNEDTATYFCARFTSPDYWGQGTTVTVSSGGGGSEVQLLESGGGLVQPGGSLKLSCAASGFDFSRYWMSWVRQAPGKGLEWIGEINPDSSTINYTPSLKDRFIISRDNAKNTLYLQMSKVRSEDTALYYCARLGQWGYFDYWGQGTTVTVSSGGGGSGGGGSGGGGSELVMTQSPSSLTVTAGERVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKRTTSSGHHHHHH

SEQ ID NO: 80 - DNA, 32 bp, Artificial Sequence
  Description of Artificial Sequence Synthetic oligonucleotide
atcaagcttgtggatatgttacaaaaataact

SEQ ID NO: 81 - DNA, 30 bp, Artificial Sequence
  Description of Artificial Sequence Synthetic oligonucleotide
atcaagcttgaaccaagaagttcaaattcc

SEQ ID NO: 82 - DNA, 34 bp, Artificial Sequence
  Description of Artificial Sequence Synthetic oligonucleotide
cgcggtggcggccgcttacacagtcctttgcatg

SEQ ID NO: 83 - DNA, 37 bp, Artificial Sequence
  Description of Artificial Sequence Synthetic oligonucleotide
aggtgtacactccgatatccagctgacccagtctcca

SEQ ID NO: 84 - DNA, 51 bp, Artificial Sequence
  Description of Artificial Sequence Synthetic oligonucleotide
ggagccgccgccgccagaaccaccaccacctttgatctcgagcttggtccc

SEQ ID NO: 85 - DNA, 53 bp, Artificial Sequence
  Description of Artificial Sequence Synthetic oligonucleotide
ggcggcggcggctccggtggtggtggttctcaggtsmarctgcagsagtcwgg

SEQ ID NO: 86 - DNA, 39 bp, Artificial Sequence
  Description of Artificial Sequence Synthetic oligonucleotide
aatccggaggagacggtgaccgtggtcccttggccccag

SEQ ID NO: 87 - DNA, 39 bp, Artificial Sequence
  Description of Artificial Sequence Primer
aggtgtacactccttattcaaccaagaagttcaaattcc

SEQ ID NO: 88 - DNA, 31 bp, Artificial Sequence
  Description of Artificial Sequence Primer
tcatccggacacagtcctttgcatgcagatg

SEQ ID NO: 89 - DNA, 504 bp, Artificial Sequence
  Description of Artificial Sequence Synthetic soluble NKG2D nucleotide sequence
gaattcaccatgggatggagctgtatcatcctcttcttggtagcaacagctacaggtgtacactccttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtgtccgggcatcatcaccatcatcattgagtcgac

SEQ ID NO: 90 - protein, 162 aa, Artificial Sequence
  Description of Artificial Sequence Synthetic soluble NKG2D amino acid sequence
MGWSCIILFLVATATGVHSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSGHHHHHH

SEQ ID NO: 91 - protein, 5 aa, Artificial Sequence
  Description of Artificial Sequence Synthetic Gly-Ser linker
GGGGS

SEQ ID NO: 92 - protein, 15 aa, Artificial Sequence
  Description of Artificial Sequence Synthetic Gly-Ser linker
GGGGSGGGGSGGGGS